(19) United States
(12) Patent Application Publication
(10) Pub. No.: US 2023/0293448 A1
(43) Pub. Date: Sep. 21, 2023

(54) NANOPARTICLE IMMUNOGENIC COMPOSITIONS AND VACCINATION METHODS

(71) Applicant: Yale University, New Haven, CT (US)

(72) Inventors: Akiko Iwasaki, Guilford, CT (US); Tianyang Mao, New Haven, CT (US); Benjamin Goldman-Israelow, New Haven, CT (US); W. Mark Saltzman, New Haven, CT (US); Alexandra Suberi, New Haven, CT (US); Melanie Reschke, New Haven, CT (US); Alexandra Piotrowski-Daspit, Wethersfield, CT (US); Hee Won Suh, North Haven, CT (US)

(21) Appl. No.: 18/063,582

(22) Filed: Dec. 8, 2022

Related U.S. Application Data
(60) Provisional application No. 63/287,410, filed on Dec. 8, 2021.
(60) Provisional application No. 63/290,042, filed on Dec. 15, 2021.
(60) Provisional application No. 63/292,200, filed on Dec. 21, 2021.
(60) Provisional application No. 63/301,942, filed on Jan. 21, 2022.
(60) Provisional application No. 63/302,413, filed on Jan. 24, 2022.
(60) Provisional application No. 63/418,744, filed on Oct. 24, 2022.

(51) Int. Cl. A61K 9/51; A61K 39/215; A61P 31/14

(52) U.S. Cl. CPC A61K 9/5146; A61K 39/215; A61P 31/14; A61K 9/5153; A61K 2039/53

(57) ABSTRACT

Compositions and methods for inducing a protective mucosal immunity against an antigen in a subject include the step of administering to a mucosal tissue an effective amount of a vaccine composition including the antigen or polynucleotide encoding an antigen associated or encapsulated within carriers such as poly(amine-co-ester) polymers in the form of particles (e.g., solid nanoparticles formed of PACE) or PACE copolymers and/or blends. Typically, the subject has previously been exposed to the antigen, for example, by administering to the same subject via a systemic or mucosal route of administration a priming antigen. In some embodiments, the polynucleotides-based vaccines are messenger RNAs encoding a viral antigen such as a coronavirus spike protein sequence, or a portion thereof. In preferred embodiments, the vaccine composition is administered intranasally.

This application claims the benefit of and priority to U.S. Ser. No. 63/287,410 filed Dec. 8, 2021; U.S. Ser. No. 63/290,042, filed Dec. 15, 2021; U.S. Ser. No. 63/292,200, filed Dec. 21, 2021; U.S. Ser. No. 63/301,942 filed Jan. 21, 2022; U.S. Ser. No. 63/302,413, filed Jan. 24, 2022; and U.S. Ser. No. 63/418,744, filed Oct. 24, 2022; which are specifically incorporated by reference herein in their entirety.

### STATEMENT REGARDING FEDERALLY SPONSORED RESEARCH OR DEVELOPMENT

This invention was made with government support under K08AI163493 awarded by National Institutes of Health and National Institute of Allergy and Infectious Disease; and under 5UG3HL147352-03 awarded by National Institutes of Health. The government has certain rights in the invention.

### REFERENCE TO SEQUENCE LISTING

The Sequence Listing submitted Dec. 8, 2022, as a text file named “YU_8277_US_ST26.xml”, created Dec. 8, 2022, and having a size of 19,828 bytes is hereby incorporated by reference pursuant to 37 C.F.R. 1.834(c)(1).

### FIELD OF THE INVENTION

The field of the invention is generally related to compositions and methods for improved vaccines, particularly nucleic acid-based agents for administration to induce an immune response.

### BACKGROUND OF THE INVENTION

Respiratory pathogens, such as epidemic influenza viruses and coronaviruses, represent a significant and recurrent public health concern. Each year in the United States, millions of hospitalizations as well as over 35,000 deaths are due to influenza-related illness. Recently, coronaviruses have caused severe epidemic and pandemic respiratory diseases in the human population, with the Severe Acute Respiratory Syndrome Coronavirus 2 (SARS-CoV-2) prompting The World Health Organization (WHO) to declare a global pandemic in mid-March 2020.

The primary defense against these pathogens is mass vaccination of the population for the creation of “herd immunity” based on a protective antibody response in the host. Currently, most of the approved vaccines and vaccine candidates in clinical development for respiratory viruses are administered intramuscularly. While such parenteral vaccines generate good systemic immunity by inducing virus-specific IgG and T cell responses in the circulation, they produce very little memory T cells and mucosal IgA in the respiratory tract where infection is first being established and later transmitted.

Respiratory viruses invade the body through the respiratory mucosa, and are then transmitted via respiratory droplets and aerosols. Thus, vaccine strategies that aim to elicit protective immune effector mechanisms at these sites are much needed to provide front-line protection against respiratory viral invasion and dissemination and may serve as a transmission blocking vaccine strategy. Strong mucosal cellular and humoral immune responses have the potential to induce sterilizing immunity by impeding pathogen binding to and uptake across epithelial surfaces. However, there are significant hurdles to mucosal vaccine development.

Therefore, it is an object of the invention to provide improved vaccines and immunization strategies which can provide effective protection against respiratory pathogens, especially corona viruses such as SARS and COVID-19 and influenza viruses.

It is an object of the invention to provide improved vaccine reagents and methods of use thereof which can provide protective mucosal immunity to respiratory pathogens.

It is a further object of the invention to convert systemic immunity induced by parenteral immunization into protective immunity to respiratory infection.

It is also an object of the invention to provide reagents and methods for effective immunization against pandemic SARS and influenza viruses.

### SUMMARY OF THE INVENTION

Immunogenic compositions and methods for inducing a mucosal immune response and/or a long lasting mucosal immunity against respiratory virus and/or boosting an existing immunity against the virus, are provided. In one embodiment, the compositions include a viral antigen in a form suitable for mucosal delivery. The viral antigen can be a protein or peptide, or polynucleotide encoding a viral antigen, provided in/on a carrier.

In a preferred embodiment, the compositions include polynucleotides-based antigen associated or encapsulated within poly(amine-co-ester) polymers in the form of micellular particles or solid polymeric nanoparticles, which are effective in inducing a mucosal immune response and/or a long lasting and protective mucosal immunity specific to the antigen, in a subject. In other embodiments, the nucleic acid is provided in liposomal, lipid nanoparticle, lipid emulsion or organogel formulations.

In some embodiments, the formulations are administered to boost or enhance an existing immunity against an infection by a virus in a subject. In some embodiments, the virus is one that causes a respiratory infection. In some embodiments, the virus is a coronavirus, an influenza virus, a herpes simplex virus, or any combinations thereof.

Methods of enhancing mucosal immunity to an antigen in a subject include the step of administering to a mucosal tissue in the subject an effective amount of a vaccine composition comprising the antigen or poly(amine-co-ester) polymers and polynucleotide encoding antigen. The subject preferably has previously been exposed to the antigen, for example, by administering to the same subject via a systemic or mucosal route of administration a priming antigen, which shares one or more antigenic sites with the antigen (or antigen encoded by the polynucleotide). Therefore, in some embodiments, the subject's immune system has been “primed” by prior exposure to the antigen. In some embodiments, the prior exposure is systemic exposure, for example, prior administration of a vaccine via a systemic route to the subject, for example, via intramuscular or intradermal administration. In other embodiments, the prior exposure is mucosal exposure, for example, prior administration of a mucosal vaccine to the subject, or a prior natural infection at the mucosal site.

In some embodiments, the prior exposure via a systemic or mucosal route of administration does not induce a protective mucosal immunity. In some embodiments, the prior exposure via a systemic or mucosal route of administration does not induce detectable number of CD8⁺ tissue-resident memory (T_{RM}) cells, CD4⁺ tissue-resident memory (T_{RM}) cells, and/or memory B cells against the antigen, at the mucosal tissue. In some embodiments, the prior exposure via a systemic or mucosal route of administration does not induce detectable mucosal IgA against the antigen. In preferred embodiments, the priming antigen includes a messenger RNA (mRNA) encoding an antigen which is associated with or encapsulated within a lipid nanoparticle.

Exemplary mucosal tissue for administering the disclosed compositions are pulmonary, nasal, oral, gastrointestinal, vaginal, and rectal mucosa. In preferred embodiments, the mucosal tissue is pulmonary and/or nasal mucosa. Suitable antigens are derived from or raise a protective immune response against one or more respiratory viruses, for example, orthomyxoviruses, paramyxoviruses, coronaviruses, adenoviruses, herpesviruses, and human bocaviruses.

The antigen is derived from, or raises a protective immune response against, one or more of influenza viruses, parainfluenza viruses, measles viruses (Measles morbillivirus), mumps viruses, and respiratory syncytial virus (RSV), human metapneumovirus, severe acute respiratory syndrome (SARS) virus, herpes simplex virus (HSV), cytomegalovirus (CMV), Epstein-Barr virus (EBV), and varicella-zoster virus (VZV). In preferred embodiments, the antigen is derived from an influenza virus, a SARS-Cov-2 virus, or RSV. Typically, the polynucleotide encoding the antigen is a deoxyribonucleic acid (DNA) or a ribonucleic acid (RNA) molecule. In preferred embodiments, the polynucleotide encoding the antigen is a messenger RNA (mRNA), including an open reading frame encoding a coronavirus spike protein sequence, or a portion thereof. In some embodiments, the coronavirus is a variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant).

An effective amount of the immunogenic composition induces a mucosal immune response and/or a long lasting and protective mucosal immunity specific to the antigen in a subject. In some embodiments, the immunogenic composition is administered in an amount effective to induce one or more of CD8⁺ tissue-resident memory (T_{RM}) cells, CD4⁺ tissue-resident memory (T_{RM}) cells, and/or memory B cells against the antigen, at the mucosal tissue. In preferred embodiments, the immunogenic composition induces an increased number of CD8⁺ tissue-resident memory (T_{RM}) cells, CD4⁺ tissue-resident memory (T_{RM}) cells, and/or memory B cells against the antigen, at the mucosal tissue, compared to an immunogenic composition delivered in the absence of poly(amine-co-ester) polymers via the same route of administration. In some embodiments, the immunogenic composition is administered in an amount effective to induce mucosal Immunoglobulin A (IgA) and/or Immunoglobulin G (IgG) against the antigen. In preferred embodiments, the immunogenic composition induces an increased quantity of mucosal IgA and/or mucosal IgG against the antigen compared to an immunogenic composition delivered in the absence of poly(amine-co-ester) polymers via the same route of administration. Exemplary mucosal IgA is secretory IgA, for example, detected in the mucosal lavage fluid selected from bronchoalveolar lavage fluid, intestinal lavage fluid, gut lavage fluid, and vaginal lavage fluid.

Methods of enhancing mucosal immunity to the repeated presentation of an antigen in a subject include the steps of: (i) administering via a systemic or mucosal route of administration an effective amount of a priming antigen; and (ii) subsequently administering intranasally to the subject an effective amount of a boosting composition containing (i) the antigen or (ii) a poly(amine-co-ester) or poly(amine-co-amide), (PACE), PACE-PEG block copolymer and/or PACE polymer blend which may include additional monomers to make the nanoparticles more acid sensitive, and the antigen, or a polynucleotide encoding the same antigen. In one embodiment, the methods include steps of: (i) administering via intramuscular (IM) injection an effective amount of a priming antigen containing lipid nanoparticles (LNP) and a polynucleotide encoding the antigen; and (ii) subsequently administering intranasally (IN) to the subject an effective amount of a boosting composition containing the antigen or a poly(amine-co-ester) or poly(amine-co-amide), (PACE), PACE-PEG block copolymer and/or PACE polymer blend which may include additional monomers to make the nanoparticles more acid sensitive, and an antigen or a polynucleotide encoding an antigen. In another embodiment, the methods include the step of administering intranasally (IN) an effective amount of a priming antigen containing poly(amine-co-ester) or poly(amine-co-amide), (PACE), PACE-PEG block copolymer and/or PACE polymer blend which may include additional monomers to make the nanoparticles more acid sensitive, and a polynucleotide encoding the antigen; and (ii) subsequently administering intranasally (IN) to the subject an effective amount of a boosting composition containing poly(amine-co-ester) or poly(amine-co-amide), (PACE), PACE-PEG block copolymer and/or PACE polymer blend which may include additional monomers to make the nanoparticles more acid sensitive, and a polynucleotide encoding the same antigen.

Methods of providing a protective immunity to one or more respiratory pathogens in a subject include the steps of: (i) administering via a systemic route of administration an effective amount of a priming immunogenic composition and subsequently ii) administering via a systemic or preferably a mucosal route to the subject an effective amount of a boosting immunogenic composition containing the antigen or a poly(amine-co-ester) or poly(amine-co-amide), (PACE), PACE-PEG block copolymer and/or PACE polymer blend which may include additional monomers to make the nanoparticles more acid sensitive, and a polynucleotide encoding the same antigen. Exemplary systemic routes of administration include intramuscular and intradermal administration. In one embodiment, the methods include steps of: (i) administering via intramuscular (IM) injection an effective amount of a priming antigen containing lipid nanoparticles (LNP) and a polynucleotide encoding the antigen; and (ii) subsequently via intramuscular (IM) injection to the subject an effective amount of a boosting composition containing poly(amine-co-ester) or poly(amine-co-amide), (PACE), PACE-PEG block copolymer and/or PACE polymer blend which may include additional monomers to make the nanoparticles more acid sensitive, and a polynucleotide encoding the same antigen. In some embodiments, the priming antigen comprises a messenger RNA (mRNA) encoding the antigen associated or encapsulated within a lipid nanoparticle. Exemplary mucosal routes of administration include the nasal or pulmonary system, or administered to a mucosal surface (vaginal, rectal, buccal, sublingual). In some embodiments, the composition for mucosal or intranasal administration is via aqueous suspension, respiratory droplets, or aerosols. In some embodiments, the PACE polymers and the polynucleotide encoding the antigen are in the form of particles, optionally the PACE polymers are polyethylene glycol (PEG)-conjugated PACE polymers.

In some embodiments, the PACE polymer contains a polymer of Formula I:

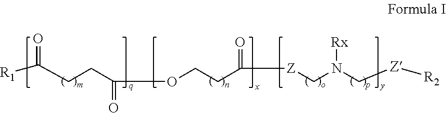

wherein n is an integer from 1-30, m, o, and p are independently integers from 1-20, x, y, and q are independently integers from 1-1000, R is hydrogen, substituted or unsubstituted alkyl, or substituted or unsubstituted aryl, or substituted or unsubstituted alkoxy, Z and Z′ are independently O or NR′, wherein R′ is hydrogen, substituted or unsubstituted alkyl, or substituted or unsubstituted aryl,
wherein R₁ and R₂ are absent or are chemical entities containing a hydroxyl group, a primary amine group, a secondary amine group, a tertiary amine group, or combinations thereof. The PACE polymers with higher lactone content are used to form solid nanoparticles.

Pharmaceutical compositions and kits for use in methods of enhancing mucosal immunity to an antigen are also provided.

### BRIEF DESCRIPTION OF THE DRAWINGS

FIGS. 1A-1N show that IN boosting with stabilized SARS-CoV-2 spike induces mucosal humoral memory. FIG. 1A is an experimental schema. Mice were intramuscularly immunized with 1 μg of mRNA-LNPs encoding full-length SARS-CoV-2 (SCV2) spike protein (Pfizer/BioNTech BNT162b2), followed by IN immunization with 1 μg of prefusion-stabilized (Hexapro), trimeric, recombinant SCV2 spike protein 14 days after mRNA-LNP immunization. Fourteen days after IN boost, serum, BALF, and nasal washes were collected to assess binding and neutralizing antibody responses. Lung tissues were collected for extravascular B cell analysis. FIGS. 1B-1G shows measurement of SCV2 spike S1 subunit-specific (FIG. 1B) nasal wash IgA, (FIG. 1C) nasal wash IgG, (FIG. 1D) BALF IgA, (FIG. 1E) BALF IgG, (FIG. 1F) serum IgA, and (FIG. 1G) serum IgG in naïve mice, mice immunized with mRNA-LNP IM (IM Prime), mice immunized with the spike protein IN (IN Spike), or mice IM primed and IN boosted with spike (P&S). FIGS. 1H-1K shows measurement of neutralization titer against SCV2 spike-pseudotyped vesicular stomatitis virus (VSV) in BALF (FIG. 1H) and serum (FIG. 1K). FIGS. 1J-1N shows that using CD45 IV labeling, various extravascular (IV labeling antibody negative) B cell subsets were measured, including RBD tetramer binding B cells, IgA⁺ B_{RM} cells, IgG⁺ B_{RM} cells, IgA⁺ ASCs, and IgG⁺ ASC in lung tissues from IM Prime or P&S mice. Mean±SEM. Statistical significance was calculated by means of [(FIG. 1B) to (FIG. 1G)] one-way analysis of variance (ANOVA) or [(FIG. 1H) to (FIG. 1N)] Student's t test; *P≤0.05, **P≤0.01,***P≤0.001,****P≤0.0001. Individual data points are represented and are pooled from two or three independent experiments.

FIGS. 2A-2O shows IN boosting with stabilized SARS-CoV-2 spike induces mucosal T cell memory. K18-hACE2 mice were intramuscularly primed with 1 μg mRNA-LNP and 14 days later intranasally boosted with 1 μg SCV2 spike. Lung tissues, BALF, and nasal turbinates were collected for extravascular T cell analysis. Lung tissues were collected 14 days after boost, whereas BALF and nasal turbinates were obtained 7 days after boost. FIGS. 2A-2I show extravascular CD8 T cell responses. Shown are quantification of SCV2 spike-specific tetramer⁺ CD8 T cells, CD69⁺CD103⁻tetramer⁺ CD8 T cells, or CD69⁺CD103⁺tetramer⁺ CD8 T cells in [(FIG. 2A) to (FIG. 2C)] lung tissues, [(FIG. 2D) to (FIG. 2F)] BALF, or [(FIG. 2G) to (FIG. 2I)] nasal turbinate from naïve, IM prime, IN spike, or P&S mice. FIGS. 2J-2O show extravascular CD4 T cell responses. Shown are quantification of activated polyclonal CD4 T cells, CD69⁺CD103⁻ CD4 T cells, or CD69⁺CD103⁺ CD4 T cells in [(FIG. 2J) to (FIG. 2L)] lung tissues or [(FIG. 2M) to (FIG. 2O)] BALF from naïve, IM prime, IN spike, or P&S mice. Mean±SEM. Statistical significance was calculated by means of [(FIG. 2B) to (FIG. 2O)] one-way ANOVA followed by Tukey's correction; *P≤0.05, **P≤0.01,***P≤0.001,****P≤0.0001. Individual data points are represented and are pooled from two or three independent experiments.

FIGS. 3A-3L show IN SARS-CoV-2 spike boosting protects against COVID-19-like disease. FIG. 3A is an experimental schema. K18-hACE2 mice were intramuscularly primed with 0.05 μg of mRNA-LNP and intranasally boosted with 1 μg of spike 14 days after IM prime. Six weeks after boost, mice were challenged with 6×10⁴ PFU SCV2 (2019n-CoV/USA_WA1/2020). The first cohort was used to evaluate weight loss and survival up to 14 days after infection. The second cohort was used to collect lung and nasal turbinate tissues 2 days after infection for viral titer measurement. The third cohort was used to collect lung tissues 5 days after infection for histological assessment. FIGS. 3B-3D show weight loss and survival of naïve, IM prime, or P&S mice from 1 to 14 days after infection. FIGS. 3E and 3F show measurement of infectious virus titer in lung and nasal turbinate tissues at 2 days after infection by means of plaque assay. FIG. 3G shows pathology score of lung sections at 5 days after infection by means of H&E staining. FIG. 3H shows representative H&E staining results from uninfected, IM prime, or P&S mice. Scale bar, 250 mm. Sections are representative of multiple sections from at least five mice per group. FIG. 3I is an experimental schema. K18-hACE2 mice were intramuscularly primed with 0.05 μg of mRNA-LNP and intranasally boosted with 10 μg of mRNA encapsulated by PACE (IN PACE-Spike) 14 days after IM Prime. Six weeks after boost, mice were challenged with 6×10⁴ PFU SCV2 (2019n-CoV/USA_WA1/2020). Weight loss and survival were monitored up to 14 days after infection. FIGS. 3J-3L show weight loss and survival of naïve, IM prime, or prime and PACE-spike K18-hACE2 mice from 1 to 14 days after Infection. Mean±SEM. Statistical significance was calculated by means of [(FIG. 3D) and (FIG. 3L)] log-rank Mantel-Cox test, [(FIG. 3E) and (FIG. 3F)] one-way ANOVA followed by Tukey's correction, or (FIG. 3G) Student's t test; *P≤0.05, **P≤0.01, ***P≤0.001, ****P≤0.0001. Individual data points are represented and are pooled from two independent experiments.

FIGS. 4A-4U show IN spike boosting elicits enhanced mucosal immunity with similar systemic humoral responses to IM mRNA-LNP boosting. FIG. 4A experimental schema. K18-hACE2 mice were IM primed with 1 μg of mRNA-LNP, followed 14 days later by boosting with 1 μg of mRNA-LNP IM or 1 μg of SCV2 spike IN. Forty-five days after prime, lung tissues were collected for T cell analysis by means of flow cytometry, and BALF and serum were collected for antibody measurement. K18-hACE2 mice were intramuscularly primed with 0.05 μg of mRNA-LNP, followed 14 days later by boosting with 0.05 μg of mRNA-LNP intramuscularly, or 1 μg of SCV2 spike intranasally and challenged with 6×10⁴ PFU SCV2 at 118 days after prime. FIGS. 4B-4D show quantification of total tetramer⁺ CD8 T cells, CD69⁺CD103⁻tetramer⁺ CD8 T cells, or CD69⁺CD103⁺tetramer⁺ CD8 T cells in lung tissues from naïve, mRNA-LNP prime-boost, or P&S mice. FIGS. 4E and 4F show quantification of total tetramer+ CD4 T cells or CD69⁺CD103⁻tetramer⁺ CD4 T cells in lung tissues. FIGS. 4G-4K show lung lymphocytes were isolated by means of Percoll gradient and restimulated with spike peptide megapool from SCV2. Intracellular cytokine staining was performed to assess antigen-specific production of TNF-α, IL-2, IFN-γ, IL-17, and IL-4 by extravascular IV⁻CD45⁻CD44⁺ CD4 T cells. FIGS. 4L-4O show measurement of SCV2 spike S1 subunit-specific (FIG. 4L) BALF IgA, (FIG. 4M) BALF IgG, (FIG. 4N) serum IgA, and (FIG. 4O) serum IgG in naïve, mRNA-LNP prime-boost, or P&S mice. FIG. 4P shows measurement of neutralization titer against SCV2 spike-pseudotyped VSV. FIGS. 4Q-4S show weight loss, survival, and disease-free survival (<5% maximum weight loss) of mRNA-LNP prime-boost or P&S mice from 1 to 14 days after infection. FIGS. 4T and 4U show measurement of infectious virus titer in lung and nasal turbinate tissues at 2 days after infection by means of plaque assay. To reduce overall number of experimental animals used, control data points from naïve and mRNA prime-boost are common to FIGS. 4 and 6. Mean±SEM. Statistical significance was calculated by means of [(FIG. 4B) to (FIG. 4O)] one-way ANOVA followed by Tukey's correction or [(FIG. 4P), (FIG. 4T), and (FIG. 4U)] Student's t test, and [(FIG. 4R) and (FIG. 4S)] log-rank Mantel-Cox test; *P≤0.05, **P≤0.01, ***P≤0.001, ****P≤0.0001. Individual data points are represented and are pooled from two independent experiments.

FIGS. 5A-5O show IN spike boosting leads to reduced viral transmission in hamster model. FIG. 5A is an experimental schema. Syrian hamsters were intramuscularly primed with 0.5 μg of mRNA-LNP, followed 21 days later by boosting with 0.5 μg of mRNA-LNP intramuscularly or 5 μg of SCV2 spike intranasally. (FIGS. 5B and 5C) Sixty-seven days after prime, serum IgG and IgA were assessed by means of ELISA. At 93 days after prime, naïve, mRNA-LNP prime-boost, and P&S hamsters were infected with 6×10³ PFU SCV2. FIG. 5D shows weight loss as percent of starting. FIG. 5D shows histopathologic analysis of lung samples at 7 days after infection. FIGS. 5F-5H shows viral titer from oropharyngeal swabs are shown as mean (symbols) and standard deviation (shaded regions), P value relative to control at the same time point. FIG. 5I shows AUC analysis for viral titer over 6 days after infection. (FIG. 5J) Transmission experimental schema. Syrian hamsters vaccinated as above were cohoused for 4 hours with naïve donor hamsters that had been infected 24 hours earlier with 6×10³ PFU SCV2. (FIG. 5K) Histopathologic analysis of lung samples at 7 days after exposure. (FIGS. 5L-5N) Viral titers from oropharyngeal swabs are shown as mean (symbols) and standard deviation (shade), P value relative to control at the same time point. (FIG. 5O) AUC analysis for viral titer over 6 days after infection. Mean±SEM. Statistical significance was calculated by means of [(FIG. 5B), (FIG. 5C), (FIG. 5E), (FIG. 5I), (FIG. 5K), and (FIG. 5O)] one-way ANOVA followed by Tukey's correction, [(FIG. 5F) to (FIG. 5H)] mixed-effect analysis followed by Tukey's multiple comparison test, or [(FIG. 5L) to (FIG. 5N)] two-way ANOVA followed by Dunnett's multiple comparisons test; *P≤0.05, **P≤0.01, ***P≤0.001, ****P≤0.0001. Individual data points are represented from one independent experiment.

FIGS. 6A-6X show heterologous IN boosting with SARS-CoV-1 spike enhances preexisting SCV2-specific immunity and broadens reactivities to SCV1. (FIG. 6A) Experimental schema. K18-hACE2 mice were intramuscularly primed with 1 μg of mRNA-LNP, followed by boosting with 1 μg of mRNA-LNP intramuscularly, or 5 μg of prefusion-stabilized, trimeric, recombinant SARS-CoV-1 (SCV1) spike IN (IN SpikeX) 14 days after prime. FIGS. 6B-6D show Quantification of total tetramer⁺ CD8 T cells, CD69⁺CD103⁻tetramer⁺ CD8 T cells, or CD69⁺CD103⁺tetramer⁺ CD8 T cells in lung tissues from naïve, mRNA-LNP prime-boost, or P&Sx mice. (FIGS. 6E-6N) Percoll gradient purified lung lymphocytes were restimulated with spike peptide megapool from [(FIG. 6E) to (FIG. 6I)] SCV1 or [(FIG. 6J) to (FIG. 6N)] SCV2, and intracellular cytokine staining was performed to assess antigen-specific production of TNF-α, IL-2, IFN-γ, IL-17, and IL-4 by extravascular IV-CD45⁻CD44⁺ CD4 T cells. FIGS. 6O-6S show Measurement of SCV1 spike S1 subunit specific BALF IgA and IgG, and serum IgA and IgG. (FIG. 6S) Measurement of neutralization titer against SCV1 spike-pseudotyped VSV. FIGS. 6T-6W show Measurement of SCV2 spike S1 subunit specific BALF IgA and IgG, and serum IgA and IgG. FIG. 6X show Measurement of neutralization titer against SCV2 spike-pseudotyped VSV. To reduce overall number of experimental animals used, control data points from naïve and mRNA prime boost are common to FIGS. 4A-4U and FIGS. 6A-6X. Mean±SEM. Statistical significance was calculated by means of one-way ANOVA followed by Tukey's correction, except for [(FIG. 6S) and (FIG. 6X)] Student's t test; *P≤0.05, **P≤0.01, ***P≤0.001, ****P≤0.0001. Individual data points are represented and are pooled from two independent experiments.

FIGS. 7A-7I show IN spike boost mediated mucosal immunity is not affected by host genotype, boosting interval or intranasal volume. (FIG. 7A) Experimental schema: K18-hACE2 or C57BL/6J (B6J) mice were IM primed with 1 μg of mRNA-LNP and IN boosted with 1 μg of SCV2 spike 2- or 4-weeks post IM Prime in 25 or 50 μl of inoculation volume. Fourteen days post boost, lung tissues were collected for T cell analysis by flow cytometry and BALF and blood were collected for antibody measurement. FIGS. 7B-7D show Quantification of total tetramer⁺ CD8 T cells, CD69⁺CD103⁻tetramer⁺ CD8 T cells, or CD69⁺CD103⁺tetramer⁺ CD8 T cells in lung tissues. FIGS. 7E-7H show Measurement of SCV2 spike S1 subunit specific BALF IgA (FIG. 7E), BALF IgG (FIG. 7F), serum IgA (FIG. 7G), and serum IgG (FIG. 7H). Measurement of neutralization titers against SCV2 spike-pseudotyped VSV (FIG. 7I) Mean±s.e.m.; Statistical significance was calculated one-way ANOVA followed by Tukey's correction (FIGS. 7B-7H); *P≤0.05, **P≤0.01, ***P≤0.001, ****P≤0.0001. Individual data points are represented and are pooled from two independent experiments.

FIGS. 8A-8K show delayed boosting with IN spike induces durable mucosal immunity. FIG. 8A show Experimental schema: K18-hACE2 mice were IM primed with 1 μg of mRNA-LNP and IN boosted with 1 μg of SCV2 spike 12 weeks post IM Prime. Lung tissues were collected for T cell analysis by flow cytometry and BALF and blood were collected for antibody measurement 7- and 56-days post boost. FIGS. 8B-8D show Quantification of total tetramer⁺ CD8 T cells (FIG. 8B), CD69⁺CD103⁻tetramer⁺ CD8 T cells (FIG. 8C), or CD69⁺CD103⁺tetramer⁺ CD8 T cells (FIG. 8D) in lung tissues from IM Prime or P&S mice 7- and 56-days post boost. (FIGS. 8E-8G) Quantification of total activated, polyclonal CD4 T cells (FIG. 8E), CD69⁺CD103⁻ CD4 T cells (FIG. 8F), or CD69⁺CD103⁺ CD4 T cells (FIG. 8G) in lung tissues from IM Prime or P&S mice 7- and 56-days post boost. FIGS. 8H-8K show Measurement of SCV2 spike S1 subunit specific BALF IgA (FIG. 8H), BALF IgG (FIG. 8I), serum IgA (FIG. 8J), and serum IgG (FIG. 8K) in IM Prime or P&S mice 7- and 56-days post boost. Mean±s.e.m.; Statistical significance was calculated two-way ANOVA followed by Tukey's correction (H to K); *P≤0.05, **P≤0.01, ***P≤0.001, ****P≤0.0001. Individual data points are represented and are pooled from two independent experiments.

FIGS. 9A-9H show IN delivery of SCV2 spike mRNA encapsulated in poly(amine-co-ester) (PACE) terpolymers mediates mucosal boosting. FIG. 9A show experimental schema: K18-hACE2 mice were IM primed with 1 μg of mRNA-LNP, followed by IN boosting with 1 μg of naked mRNA (IN naked mRNA) or 1 μg of mRNA encapsulated by PACE (IN PACE-Spike) 14 days post IM Prime. Fourteen days post IN boost, BALF and blood were collected for antibody measurement. Lung tissues were collected for CD8 T cell analysis. FIGS. 9B-9D show quantification of total tetramer⁺ CD8 T cells (FIG. 9B), CD69⁺CD103⁻tetramer⁺ CD8 T cells (FIG. 9C), or CD69⁺CD103⁺tetramer⁺ CD8 T cells (FIG. 9D) in lung tissues from naïve, IM Prime, IN PACE-Spike, IM Prime+IN naked mRNA, or Prime and PACE-Spike mice. FIGS. 9E-9H show measurement of SARS-CoV-2 spike S1 subunit-specific BALF IgA (FIG. 9E), BALF IgG (FIG. 9F), serum IgA (FIG. 9G), and serum IgG (FIG. 9H) in naïve, IM Prime, IN PACE-Spike, IM Prime+IN naked mRNA, or Prime and PACE-Spike mice. Mean±s.e.m.; Statistical significance was calculated by one-way ANOVA followed by Tukey's correction (FIGS. 9B-9D) or two-way ANOVA followed by Tukey's correction (FIGS. 9E-9H); *P≤0.05, **P≤0.01, ***P≤0.001, ****P≤0.0001. Data are pooled from two independent experiments.

FIGS. 10A-10H show IN spike boost-induced protection against COVID-19 correlates with enhanced mucosal immunity. FIG. 10A show experimental schema: K18-hACE2 mice were IM primed with 0.05 μg of mRNA-LNP and IN boosted with 1 μg of spike IN 14 days post IM Prime. Six weeks post boost, lung tissues were collected for CD8 T cell analysis by flow cytometry, and BALF and blood were collected for antibody measurement. FIGS. 10B-10D show quantification of total tetramer+ CD8 T cells, CD69⁺CD103⁻tetramer⁺ CD8 T cells, or CD69⁺CD103⁺tetramer⁺ CD8 T cells in lung tissues from naïve, IM Prime, or P&S mice. FIGS. 10E-10H show measurement of SCV2 spike S1 subunit specific BALF IgA (FIG. 10E), BALF IgG (FIG. 10F), serum IgA (FIG. 10G), and serum IgG (FIG. 10H) in naïve, IM Prime, or P&S mice. Mean±s.e.m.; Statistical significance was calculated one-way ANOVA followed by Tukey's correction (FIGS. 10B-10D) or two-way ANOVA followed by Tukey's correction (FIGS. 10E-10H); *P≤0.05, **P≤0.01, ***P≤0.001, ****P≤0.0001. Individual data points are represented from one independent experiment.

FIGS. 11A-11G show MHC-peptide tetramer-based identification of SARS-CoV-2 spike-specific CD8 and CD4 T cells. FIG. 11A show sequence alignment of CD4 epitope (VTWFHAIHVSGTNGT (SEQ ID NO:6), Spike₆₂₋₇₆) and CD8 epitope (VNFNFNGL (SEQ ID NO: 6), Spike₅₃₉₋₅₄₆) from spike proteins of sarbecoviruses. MHC Class II Sequences are shown as SEQ ID NO:8-17 and MHC Class I Sequences are shown as SEQ ID NO:18-22. FIGS. 11B-11D show identification and phenotypic analysis of spike-specific CD8 T cells (FIG. 11C) and CD4 T cells (FIG. 11D) in the lung 42 days post IM Prime and 28 days following IN spike boost. FIGS. 11E-11G show identification and phenotypic analysis of spike-specific CD8 T cells (FIG. 11F) and CD4 T cells (FIG. 11G) in the lung 14 days following SARS-CoV-2 infection. Contour plots (FIGS. 11C and 11F) are representative of two independent experiments.

FIGS. 12A-12E show IN spike boost mediates expansion of polyfunctional spike specific CD4 T cells in the lung parenchyma. FIG. 12A show experimental schema: K18-hACE2 mice were IM primed with 1 μg of mRNA-LNP and IN boosted with 1 μg of spike IN or IM boosted with 1 μg of mRNA-LNP 14 days post IM Prime. Forty-five days post prime, lung tissues were collected for assessment of antigen specific CD4⁺ T cell cytokine production by flow cytometry. Percoll gradient purified lung lymphocytes were restimulated with spike peptide megapool from SARS-CoV-2 and intracellular cytokine staining was performed to assess cytokine production by extravascular IV-CD45⁻CD4⁺CD44⁺ T cells in naïve, mRNA-LNP prime-boost, or P&S mice. FIGS. 12B-12E show assessment of co-production of TNF-α and IL-2 (FIG. 12B), TNF-α and IFN-γ (FIG. 12C), IL-2 and IFN-γ (FIG. 12D), or TNF-α, IL-2, and IFN-γ (FIG. 12E). Mean±s.e.m.; Statistical significance was calculated one-way ANOVA followed by Tukey's correction (FIGS. 12B-12E); *P≤0.05, **P≤0.01, ***P≤0.001, ****P≤0.0001. Individual data points are represented and are pooled from two independent experiments.

FIGS. 13A-13H show IN spike boost mediates expansion of polyfunctional spike-specific CD4 T cells in the lung parenchyma. FIGS. 13A-13H experimental schema: K18-hACE2 mice were IM primed with 1 μg of mRNA-LNP and IN boosted with 5 μg of SARS-CoV-1 spike or IM boosted with 1 μg of mRNA-LNP 14 days post IM Prime. Forty-five days post prime, lung tissues were collected for assessment of antigen specific CD4 T cell cytokine production by flow cytometry. Percoll gradient-purified lung lymphocytes were restimulated with spike peptide megapool from SARS-CoV-1 (FIGS. 13A-13D) or SARS-CoV-2 (FIGS. 13E-13H) and intracellular cytokine staining was performed to assess cytokine production by extravascular IV-CD45⁻CD44⁺ CD4 T cells in naïve, mRNA prime-boost, or P&Sx mice. FIGS. 13A-13D show assessment of co-production of TNF-α and IL-2 (FIG. 13A), TNF-α and IFN-γ (FIG. 13B), IL-2 and IFN-γ (FIG. 13C), or TNF-α, IL-2, and IFN-γ (FIG. 13D) by extravascular SARS-CoV-1 spike-specific CD4⁺ T cells. (FIGS. 13E-13H) Assessment of co-production of TNF-α and IL-2 (FIG. 13E), TNF-α and IFN-γ (FIG. 13F), IL-2 and IFN-γ (FIG. 13G), or TNF-α, IL-2, and IFN-γ (FIG. 13H) by extravascular SARS-CoV-2 spike-specific CD4⁺ T cells. Mean±s.e.m.; Statistical significance was calculated one-way ANOVA followed by Tukey's correction (FIGS. 13A-13H); *P≤0.05, **P≤0.01, ***P≤0.001, ****P≤0.0001. Individual data points are represented and are pooled from two independent experiments.

FIG. 14 shows confirmation of integrity of mRNA extracted from Comirnaty mRNA-LNP. The length and integrity of extracted mRNA was analyzed using agarose gel electrophoresis. Extracted mRNA was mixed with SYBR Safe stain before being loaded onto a 1% agarose gel, let run in the TAE buffer, and imaged with a gel imaging system.

FIGS. 15A-15C show gating strategies for analysis of extravascular adaptive immune responses in the respiratory tract. FIG. 15A show gating strategies to identify extravascular spike-specific CD8 and CD4 T cells. FIG. 15B show gating strategies to identify extravascular antigen-specific and polyclonal B cells. FIG. 15C show gating strategies to identify cytokine-producing extravascular spike-specific CD4 T cells.

FIG. 16 is a bar graph of PEG-conjugated PACE-mediated delivery and expression of mRNA cargoes into the lung, showing RLU/mg protein (0-10,000) over % PACE-PEG content (0-50%) for each of control and samples containing 0%, 0.25%, 1%, 5%, 10%, or 50% PACE-PEG, respectively.

FIG. 17 is a schematic showing the methodologies using mRNA-PACE as a mucosal boost agent to establish tissue-resident immunity against SARS-CoV-2. The bottom panel is a schematic diagram for the method applied in a K18-hACE2 mouse model, including Priming step at Day −14 (injecting mRNA-LNP (lipid nanoparticles) at 1 microgram RNA via intramuscular (IM) route); Boosting step at Day 0 (injecting mRNA-PACE particles at 1 microgram RNA, via intramuscular (IM) or intranasal (IN) route; 2 weeks post-Priming); and immunophenotypic analysis at Day 14 (which is consisted of IV antibody labelling followed by lung, bronchoalveolar lavage fluid (BALF) and serum collection; 2 weeks post Boosting), respectively. The top right panel is a schematic representation of the process for IV antibody labelling and flow cytometric analysis.

FIGS. 18A-18C are bar graphs of IM mRNA-LNP prime and IN mRNA-PACE boost-induced lung-resident (IV⁻) spike-specific CD8 T cell immunity, showing Absolute cell number (#10⁰-10⁵) for each of samples of Naïve (●); IM LNP (▴); IM LNP>IN naked mRNA ( [symbol] ); IN PACE (♦); IM LNP>IM PACE ( [symbol] ); and IM LNP>IN PACE (▾) cells, respectively, acquired with/without (+/−) each of IM mRNA-LNP Prime; IN naked mRNA Boost; IM mRNA-PACE Boost and IN mRNA-PACE Boost, respectively, for each of total spike-specific (IV⁻Tetramer⁺CD8⁺T cells) (FIG. 18A); antigen-experienced, activated (IV⁻Tetramer⁺CD69⁺CD8⁺T cells) (FIG. 18B); and Tissue resident memory (T_{RM}) (IV⁻Tetramer⁺CD69⁺CD103⁺CD8⁺T cells) (FIG. 18C) cells, respectively.

FIGS. 19A-19C are bar graphs showing that IM mRNA-LNP prime followed by IN mRNA-PACE boost did not affect circulating (IV+) CD8 T cell responses, with graphs showing Absolute cell number (#10⁰-10⁵) for each of samples of Naïve (●); IM LNP (▴); IM LNP>IN naked mRNA ( [symbol] ); IN PACE (♦); IM LNP>IM PACE ( [symbol] ); and IM LNP>IN PACE (▾) cells, respectively, acquired with/without (+/−) each of IM mRNA-LNP Prime; IN naked mRNA Boost; IM mRNA-PACE Boost and IN mRNA-PACE Boost, respectively, for each of total spike-specific (IV⁺Tetramer⁺CD8⁺T cells) (FIG. 19A); antigen-experienced, activated (IV⁺Tetramer⁺CD69⁺CD8⁺T cells) (FIG. 19B); and tissue resident memory (IV⁺Tetramer⁺CD69⁺CD103⁺CD8⁺T cells) (FIG. 19C) cells, respectively.

FIGS. 20A-20D are graphs showing that IM mRNA-LNP prime followed by IN mRNA-PACE boost elicited robust mucosal and systemic spike-specific IgA and IgG, with graphs showing optical density (OD) of anti S1 IgA (1-4) for each of samples of Naïve (●); IM LNP (▴); IN PACE (♦); IM LNP>IM PACE ( [symbol] ); and IM LNP>IN PACE (▾) cells, respectively, obtained from BALF at dilutions 1:36 (FIG. 20A) or 1:216 (FIG. 20B), and Serum at dilutions 1:1250 (FIG. 20C) or 1:62500 (FIG. 20D), respectively.

FIG. 21 is a schematic diagram for the method applied in a K18-hACE2 mouse model, including Priming step at Day 0 (injecting mRNA-LNP (lipid nanoparticles) at 0.05 microgram RNA via intramuscular (IM) route); Boosting step at Day 14 (injecting mRNA-PACE particles at 10 microgram RNA, via intramuscular (IM) or intranasal (IN) route; 2 weeks post-Priming); pre-infection bleed at Day 49; IN SARS-CoV-2 challenge at Day 56; and two weeks of monitoring of weight loss and survival until Day 70.

FIGS. 22A-22C are line graphs showing O.D. anti S1 IgG as a function of log dilution of 2-5 (FIG. 22A), % weight at Day 0 over a period of 14 days from Day 0 to Day 14 post infection (FIG. 22B), and % survival over a period of 14 days from Day 0 to Day 14 post infection (FIG. 22C) in each of samples of Naïve (●); IM Prime (▴); IM Prime>IM PACE ( [symbol] ); and IM Prime>IN PACE (▾).

FIG. 23 is a schematic diagram for the method applied in a K18-hACE2 mouse model, including Priming step at Day 0 (administration of mRNA-PACE particles at 10 microgram RNA via intranasal (IN) route); Boosting step at Day 28 (administration of mRNA-PACE particles at 10 microgram RNA via intranasal (IN) route; 4 weeks post-Priming); and immunophenotypic analysis at Day 42 (which is consisted of IV antibody labelling followed by lung, bronchoalveolar lavage fluid (BALF) and serum collection; 2 weeks post Boosting), respectively.

FIGS. 24A-24F are bar graphs of IN mRNA-PACE prime and IN mRNA-PACE boost-induced tissue-resident (IV⁻) spike-specific CD8 T cell immunity, showing Absolute cell number in the lung of Naïve (●) and IN PACE>IN PACE (▴) groups for each of total spike-specific (IV⁻Tetramer⁺CD8⁺T cells) (FIG. 24A); antigen-experienced, activated (IV⁻Tetramer⁺CD69⁺CD8⁺T cells) (FIG. 24B); and Tissue resident memory (T_{RM}) (IV⁻Tetramer⁺CD69⁺CD103⁺CD8⁺T cells) (FIG. 24C) cells, respectively; and Absolute cell number in the mediastinal lymph node of Naïve (●) and IN PACE>IN PACE (▴) groups for each of total spike-specific (IV⁻Tetramer⁺CD8⁺T cells) (FIG. 24D); antigen-experienced, activated (IV⁻Tetramer⁺CD69⁺CD8⁺T cells) (FIG. 24E); and Tissue resident memory (T_{RM}) (IV⁻Tetramer⁺CD69⁺CD103⁺CD8⁺T cells) (FIG. 24F) cells, respectively.

FIGS. 25A-25D are bar graphs of IN mRNA-PACE prime and IN mRNA-PACE boost-induced humoral immune responses in the draining lymph node, showing Absolute cell number of Naïve (●) and IN PACE>IN PACE (▴) groups for each of CXCR5⁺PD1⁺T_{FH} cells (FIG. 25A); GL7+ germinal center (GC) B cells (FIG. 25B); CD138+ antibody secreting cells (ASC) (FIG. 25C); and Tetramer+ B cells (FIG. 25D), respectively.

FIGS. 26A-26D are bar graphs showing that IN mRNA-PACE prime followed by IN mRNA-PACE boost elicited robust mucosal and systemic spike-specific IgA and IgG, with graphs showing optical density (OD) of anti S1 IgG (1-3) for each of samples of Naïve (●) and IN PACE>IN PACE (▴), obtained from BALF at dilutions 1:36 or 1:216 (FIG. 26A), and Serum at dilutions 1:1250 or 1:62500 (FIG. 26B), respectively; optical density (OD) of anti S1 IgA (1-3) for each of samples of Naïve (●) and IN PACE>IN PACE (▴), obtained from BALF at dilutions 1:6 or 1:36 (FIG. 26C), and Serum at dilutions 1:250 or 1:1250 (FIG. 26D), respectively.

FIG. 27A demonstrates that a “prime and spike” vaccination strategy induced lung-resident spike-specific CD8 T cell immunity, in accordance with some embodiments. “IV⁻” indicates that the cell is in the tissue (e.g., lung) rather than vascular in nature. “Tetramer+” indicates that the CD8 T cell is reactive with a tetrameric class I MHC protein in complex with a SARS-CoV-2 spike peptide (“VNFNFNGL” SEQ ID NO:6). “CD69+” means that the cell is CD69 positive, which indicates that the CD8 T cell is an antigen-experienced CD8 T cell. “CD69+CD103+” means the cell is both CD69 positive and CD103 positive, which indicates that the CD8 T cell is a tissue-resident memory CD8 T cell. FIG. 27B demonstrate that the “prime and spike” vaccination strategy induced expansion of lung-resident CD4 T cells, in accordance with some embodiments. “CD44+” means that the cell is CD44 positive, which indicates that the CD4 T cell is polyclonally activated. “CD69+” means that the cell is CD69 positive, which indicates that the CD4 T cell is an antigen-experienced CD4 T cell. “CD69+CD103+” means the cell is both CD69 positive and CD103 positive, which indicates that the CD4 T cell is a tissue-resident memory CD4 T cell. FIGS. 27C-27F demonstrate that the “prime and spike” vaccination strategy induced antigen-specific and polyclonal expansion of diverse extravascular B cell responses, including antibody secreting cells, resident memory B cells, and germinal center-like B cells, in accordance with some embodiments.

FIGS. 28A-28D demonstrate that the “prime and spike” vaccination strategy elicited robust airway and systemic spike protein S1 subunit-specific IgA and IgG response, in accordance with some embodiments. “BAL” means bronchoalveolar lavage fluid (BALF), which indicates that the levels of the indicated Ig were measured in the BALF. FIGS. 28E-28G demonstrate that the “prime and spike” vaccination strategy elicited upper respiratory T cell and antibody responses in the nasal cavity, based on nasal tissue and nasal wash measurements, in accordance with some embodiments.

FIGS. 29A-29C demonstrate that the “prime and spike” vaccination strategy induced much stronger mucosal T cell and antibody responses than the conventional mRNA-LNP “prime and boost” strategy. In Figs. “IV-CD44+Tetramer+CD8+ TRM” means extravascular (“IV−”), activated (“CD44+”) CD8 tissue-resident memory T cells that are reactive with a SARS-CoV-2 spike peptide (“Tetramer+”). “IV-CD44+CD4+ TRM” means extravascular (“IV−”), activated (“CD44+”) CD4 tissue-resident memory T cells. “BAL anti-spike S1 IgA” means anti-SARS-CoV-2 spike protein S1 domain IgA from the BALF. FIG. 29D demonstrates that, following extended between-dose interval, the “prime and spike” vaccination strategy induced stronger mucosal IgA response than the conventional mRNA-LNP “prime and boost” vaccination strategy, in accordance with some embodiments. FIGS. 29E-29G demonstrate that that the “prime and spike” vaccination strategy provided superior antiviral control against SARS-CoV-2 in both lower respiratory tract (lung parenchyma) and upper respiratory tract (nasal turbinate) compared to naïve mice or mice receiving IM prime only, in accordance with some embodiments. FIG. 29H shows data on mice that were IM primed and boosted with mRNA-LNP SCV2 vaccine and additionally IN boosted with SCV1 spike also developed high levels of tissue-resident memory CD8 T cells specific for a conserved epitope shared between SCV1 and SCV2 (VNFNFNGL, SEQ ID NO:6).

FIGS. 30A-30D demonstrate that mice that were IM primed and IN boosted with SCV1 spike proteins had significantly higher titers of anti-SCV1 IgA (FIGS. 30A and 30C) and IgG (FIGS. 30B and 30D) in both BALF (FIGS. 30A-30B) and serum (FIGS. 30C-30D) compared to mice that were IM primed and later boosted with IM mRNA-LNP SCV2 vaccine, IM SCV1 spike, IN SCV2 spike, or IM SCV2 spike, in accordance with some embodiments. Mice that were IM primed and boosted with mRNA-LNP SCV2 vaccine and additionally IN boosted with SCV1 spike had further increases of mucosal and circulating anti-SCV1 IgA and IgG compared to mice that were IM primed and IN SCV1 spike boosted. FIGS. 30E-30H demonstrate that mice that were IM primed and IN boosted with SCV1 or SCV2 spike proteins had significantly higher titers of anti-SCV2 IgA (FIGS. 30E and 30G) in the BALF (FIGS. 30E-30F) compared to mice that were IM primed and later boosted with IM mRNA-LNP SCV2 vaccine, IM SCV1 spike, or IM SCV2 spike, in accordance with some embodiments. Mice that were IM primed and IN SCV2 boosted developed comparable levels of BALF anti-SCV2 IgG, serum anti-SCV2 IgA, and serum anti-SCV2 IgG compared to mice that were IM primed and boosted with mRNA-LNP SCV2 vaccine. Mice that were IM “primed” and IN SCV1 “spiked” also developed robust levels of BALF anti-SCV2 IgG, serum anti-SCV2 IgA, and serum anti-SCV2 IgG. Similar to anti-SCV1 antibody response, mice that were IM primed and boosted with mRNA-LNP SCV2 vaccine and additionally IN boosted with SCV1 spike developed highest levels of mucosal and circulating anti-SCV2 IgA and IgG compared to all other groups. FIG. 30I demonstrates that mice that were IM primed and later boosted with IN SCV1 or IM SCV1 spike proteins had significantly higher serum neutralizing titers (IC50) against Vesicular Stomatitis Virus (VSV)-pseudotyped SCV1 compared to mice that were IM primed and later boosted with IM mRNA-LNP SCV2 vaccine, IM SCV2 spike, or IN SCV2 spike, in accordance with some embodiments. As shown in FIG. 22, mice that were IM primed and later boosted with IN SCV2 spike or IM mRNA-LNP SCV2 vaccine had significantly higher serum neutralizing titers (IC50) against VSV-pseudotyped SCV2 compared to mice that were IM primed and later boosted with IM SCV2 spike, IM SCV1 spike, or IN SCV1 spike. Consistent with binding antibody titers, mice that were IM primed and boosted with mRNA-LNP SCV2 vaccine and additionally IN boosted with SCV1 spike developed high serum neutralizing titers (IC50) against both VSV-pseudotyped SCV1 and SCV2.

### DETAILED DESCRIPTION OF THE INVENTION

### I. Definitions

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

The term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

The term “biodegradable” as used herein means that the material degrades or breaks down into its component subunits, or digestion, e.g., by a biochemical process, of the material into smaller (e.g., non-polymeric) subunits.

The term “construct” refers to a recombinant genetic molecule having one or more isolated polynucleotide sequences.

The term “expression control sequence” refers to a nucleic acid sequence that controls and regulates the transcription and/or translation of another nucleic acid sequence. Control sequences that are suitable for prokaryotes, for example, include a promoter, optionally an operator sequence, a ribosome binding site, and the like. Eukaryotic cells are known to utilize promoters, polyadenylation signals, and enhancers.

A disease or disorder is “alleviated” if the severity of a symptom of the disease or disorder, the frequency with which such a symptom is experienced by a patient, or both, is reduced.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term an “open reading frame” or “ORF” is a series of nucleotides that contains a sequence of bases that could potentially encode a polypeptide or protein. An open reading frame is located between the start-code sequence (initiation codon or start codon) and the stop-codon sequence (termination codon).

The phrases “parenteral administration” and “administered parenterally” are art-recognized terms, and include modes of administration other than enteral and topical administration, typically by injection, and can include intravenous, intramuscular, intrapleural, intravascular, intradermal, intraperitoneal, transtracheal, and subcutaneous injection and infusion.

The term “particles” include microparticles (including microspheres and microcapsules) (dimensions on average 1 micron to less than about 1000 microns) and nanoparticles (nanospheres and nanocapsules) (diameter less than 1 micron). A particle may be spherical or non-spherical and may have a regular or irregular shape. In certain embodiments, populations of the nanoparticles have an average diameter of about 500 nm, 200 nm, 100 nm, 50 nm, or 10 nm. In some embodiments, the average diameter of the particles is from about 200 nm to about 600 nm, preferably from about 200 to about 500 nm. The term “diameter” is used herein to refer to either of the physical diameter or the hydrodynamic diameter. The diameter of an essentially spherical particle may refer to the physical or hydrodynamic diameter. The diameter of a non-spherical particle may refer preferentially to the hydrodynamic diameter. The diameter of a non-spherical particle may refer to the largest linear distance between two points on the surface of the particle. When referring to multiple particles, the diameter of the particles typically refers to the average diameter of the particles. Particle diameter can be measured using a variety of techniques in the art including, but not limited to, dynamic light scattering.

The phrase “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

A “poly(A)” refers to a series of adenosines attached by polyadenylation to the mRNA. In the preferred embodiment of a construct for transient expression, the polyA is between 50 and 5000, preferably greater than 64, more preferably greater than 100, most preferably greater than 300 or 400. poly(A) sequences can be modified chemically or enzymatically to modulate mRNA functionality such as localization, stability or efficiency of translation.

As used herein, the term “prime” refers to immunizing a subject by administering a vaccine parenterally to elicit a systemic T and/or B cell response(s) against a viral infection in the subject. As used herein, the term “boost” refers to administering to the same subject a vaccine parenterally against the same or related viral infections after the “prime.”

The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides contain as amino acids. Amino acid sequences are written left to right in the direction from the amino to the carboxy terminus, and denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “spike” refers to administering to a subject a viral antigen such as a viral protein, or a polynucleotide encoding a viral protein, intranasally. The “spike” step often takes place after the “prime” step, but does not have to. The viral protein can be any viral proteins that are immunogenic targets for T cells and/or B cells, but is sometimes a protein exposed on a surface of the virus, such as a spike protein of an enveloped virus, such as a spike protein of a coronavirus.

The term “surfactant” as used herein refers to an agent that lowers the surface tension of a liquid.

The phrase “sustained release” refers to release of a substance over an extended period of time, in contrast to a bolus type administration in which the majority of the substance is made biologically available at one time. A “promoter site” refers to a sequence of nucleotides to which an RNA polymerase, such as the DNA-dependent RNA polymerase originally isolated from bacteriophage, described by Davanloo, et al., Proc. Natl. Acad. Sci. USA, 81:2035-39 (1984), or from another source, binds with high specificity, as described by Chamberlin, et al., Nature, 228:227-231 (1970).

The term “transient” refers to expression of a non-integrated transgene for a period of hours, days or weeks, wherein the period of time of expression is less than the period of time for expression of the gene if integrated into the genome or contained within a stable plasmid replicon in the host cell.

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides which do not significantly alter the characteristics of the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties. In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Substitution of like amino acids can also be made on the basis of hydrophilicity, particularly where the biological functional equivalent polypeptide or peptide thereby created is intended for use in immunological embodiments. Amino acid substitutions are generally based on the relative similarity of the amino acid side-chain substituents, for example, their hydrophobicity, hydrophilicity, charge, and size.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The terms “lactone” and “lactone unit” are used to describe a chemical compound that includes a cyclic ester, or the open chain chemical structure that results from the cleavage of the ester bond in the cyclic ester. For example, lactone is used to describe the cyclic ester shown below, and the corresponding lactone-derived open chain structure:

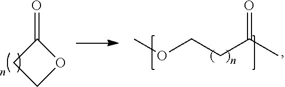

n being an integer. The open chain structure is formed via methods known in the art, including but not limited to, solvolysis, such as hydrolysis, and enzymatic cleavage.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. All integer values of the number of backbone carbon atoms between one and 30 are contemplated and disclosed for the straight chain or branched chain alkyls. Likewise, preferred cycloalkyls have from 3-10 carbon atoms in their ring structure, and more preferably have 5, 6, or 7 carbons in the ring structure. All integer values of the number of ring carbon atoms between three and 10 are contemplated and disclosed for the cycloalkyls.

The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

It will be understood by those skilled in the art that the moieties substituted on the hydrocarbon chain can themselves be substituted, if appropriate. For example, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

“Aryl” refers to C₅-C₁₀-membered aromatic, heterocyclic, fused aromatic, fused heterocyclic, biaromatic, or bihetereocyclic ring systems. In some forms, the ring systems have 3-50 carbon atoms. Broadly defined, “aryl”, as used herein, includes 5-, 6-, 7-, 8-, 9-, 10- and 24-membered single-ring aromatic groups that may include from zero to four heteroatoms, for example, benzene, naphthalene, anthracene, phenanthrene, chrysene, pyrene, corannulene, coronene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

“Alkoxy” refers to an alkyl group as defined above with the indicated number of carbon atoms attached through an oxygen bridge. Examples of alkoxy include, but not limited to, methoxy, ethoxy, n-propoxy, i-propoxy, n-butoxy, s-butoxy, n-pentoxy, s-pentoxy, and derivatives thereof.

Primary amines arise when one of three hydrogen atoms in ammonia is replaced by a substituted or unsubstituted alkyl or a substituted or unsubstituted aryl group. Secondary amines have two organic substituents (substituted or unsubstituted alkyl, substituted or unsubstituted aryl or combinations thereof) bound to the nitrogen together with one hydrogen. In tertiary amines, nitrogen has three organic substituents.

“Substituted”, as used herein, means one or more atoms or groups of atoms on the monomer has been replaced with one or more atoms or groups of atoms which are different than the atom or group of atoms being replaced. In some embodiments, the one or more hydrogens on the monomer is replaced with one or more atoms or groups of atoms. Examples of functional groups which can replace hydrogen are listed above in the definition. In some embodiments, one or more functional groups can be added which vary the chemical and/or physical property of the resulting monomer/polymer, such as charge or hydrophilicity/hydrophobicity, etc. Exemplary substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The terms “SARS-CoV-2” and “Severe Acute Respiratory Syndrome Coronavirus 2” refer to the pathogenic coronavirus strains of the subgenus Sarbecovirus which are derived from the beta-coronavirus of zoonotic origin which emerged in Asia in late 2019, and which are the causative agents of pandemic Coronavirus disease 2019 (COVID-19) in humans. SARS-CoV-2 viruses have a high rate of genetic mutation within the genome, resulting in rapid development of multiple variant SARS-CoV-2 virus strains. Multiple variants of the virus that causes COVID-19 have been documented globally during this pandemic, including a variant called B.1.1.7 identified in the United Kingdom, a variant called B.1.351 identified in South Africa, and a variant called P.1 identified in Brazil.

The terms “influenza virus”, “influenza” and “flu virus” are used interchangeably and refer to the group of influenza virus A, influenza virus B, influenza virus C and influenza virus D. Human influenza A and B viruses cause seasonal epidemics of disease (termed the “flu season”) in humans almost every winter in the United States. Global epidemics of flu disease are termed “Flu pandemics”, and typically occur when a new and very different influenza A virus emerges that both infects humans and has the ability to spread efficiently between humans. Influenza A viruses are categorized as either the hemagglutinin subtype or the neuraminidase subtype based on the proteins involved. There are 18 distinct subtypes of hemagglutinin and 11 distinct subtypes of neuraminidase. Influenza A is the primary cause of flu epidemics.

The terms “immunologic”, “immunological” or “immune” response refer to the development of a beneficial humoral (antibody mediated) and/or a cellular (mediated by antigen-specific T cells or their secretion products) response directed against an immunogen. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The term “T cell antigen” refers to a protein or fragment thereof which can be processed into a peptide that can bind to either Class I MHC, Class II MHC, non-classical MHC, or CD1 family molecules (collectively antigen presenting molecules), and in this combination can engage a T cell receptor on a T cell. Accordingly, a T cell mediated immune response is a response that occurs as a result of recognition of a T cell antigen bound to an antigen presenting molecule on the cell surface of an antigen presenting cell, coupled with other interactions between costimulatory molecules on the T cell and APC. This response serves to induce T cell proliferation, anatomic migration, and production of effector molecules, including cytokines and other factors that can injure cells.

The term “protect” or “protection of” a subject from developing a disease or from becoming susceptible to an infection means to partially or fully protect a subject from disease, infection and/or symptoms. For example, to “fully protect” means that a treated subject does not develop a disease or infection caused by a pathogenic agent, such as respiratory pathogen. To “partially protect” as used herein means that a certain subset of subjects may be fully protected from developing a disease or infection after treatment, or that the subject does not develop a disease or infection with the same severity as an untreated subject.

The terms “inhibit” or “reduce” generally mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%, or an integer there between. In some embodiments, the inhibition and reduction are compared at mRNA, protein, cellular, tissue and/or organ levels.

The terms “prevent”, “prevention” or “preventing” mean to administer a composition or method to a subject at risk for, or having a predisposition for, one or more symptom caused by a disease or disorder, to decrease the likelihood the subject will develop one or more symptoms of the disease or disorder, or to reduce the severity, duration, or time of onset of one or more symptoms of the disease or disorder.

### II. Immunogenic Compositions

The Examples demonstrated that “spiking” a subject that have existing immunity against a virus with a composition suitable for intranasally administration of a viral protein (e.g., intranasal administration of SARS-CoV-2 spike protein), and that administering the composition is able to significantly induce robust immunity, especially in the nasal and lung parenchyma and airways of the subject. In some embodiments, the virus is one that causes a respiratory infection. Accordingly, in some aspects, the present invention is directed to a composition for boosting an existing immunity against a coronavirus infection in a subject.

Immunogenic compositions containing particles (such as microparticles and/or nanoparticles) of poly(amine-co-ester) polymers and polynucleotides encoding antigens have been developed.

The composition and/or methods of the present invention are intended to be useful in the methods of present invention in combination with one or more additional compounds useful for treating the diseases or disorders contemplated within the invention. These additional compounds may comprise compounds of the present invention or compounds, e.g., commercially available compounds, known to treat, prevent, or reduce the symptoms of the diseases or disorders contemplated within the invention.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

A. Particles Containing Poly(Amine-Co-Ester)s or Poly(Amine-Co-Amide)s Modified with Polyalkylene Oxide

In one embodiment, the particles contain poly(amine-co-ester)s or poly(amine-co-amide)s (PACE) modified with a polyalkylene oxide (PACE-PAO). Preferably, the polyalkylene oxide is poly(ethylene glycol) (PEG). The PACE-PAO polymer is optionally blended with a second PACE polymer that optionally contains an endgroup modification.

1. Particles Containing PACE-PAO not Blended with a Second PACE Polymer

In some forms, the particles contain between about 0.1% wt/wt and about 95% wt/wt, between about 0.1% wt/wt and about 50% wt/wt, between about 0.1% wt/wt and about 10% wt/wt, between about 0.1% wt/wt and about 5% wt/wt of PACE-PAO, between about 1.0% wt/wt and about 5% wt/wt of PACE-PAO, such as PACE-PEG, expressed in terms of weight of the PACE-PAO polymer to total weight of the nanoparticles.

The PACE-PAO polymers have the general formula:

((A)ₓ-(B)_{y}-(C)_{q}-(D)_{w}-(E)_{f})ₕ, Formula Ia′

wherein A, B, C, D, and E independently include monomeric units derived from lactones such as pentadecalactone, a polyfunctional molecule such as N-methyldiethanolamine, a diacid or diester such as diethylsebacate, or polyalkylene oxide such as polyethylene glycol, wherein the PACE-PAO polymers include at least a lactone, a polyfunctional molecule, diacid or diester monomeric units, and polyalkylene oxide such as polyethylene glycol. In general, the polyfunctional molecule contains one or more cations, one or more positively ionizable atoms, or combinations thereof. The one or more cations are formed from the protonation of a basic nitrogen atom or from quaternary nitrogen atoms.

In general, for the PACE-PAO polymers, x, y, q, w, and f are independently integers from 0-1000, with the proviso that the sum (x+y+q+w+f) is greater than one. h is an integer from 1 to 1000.

In some forms of the PACE-PAO polymers, the percent composition of the lactone unit is between about 10% and about 100%, calculated lactone unit vs. (lactone unit+diester/diacid). Expressed in terms of a molar ratio, the lactone unit versus (lactone unit+diester/diacid) content is between about 0.1 and about 1, i.e., x/(x+q) is between about 0.1 and about 1. Preferably, in some forms of the PACE-PAO polymers, the percent composition of the lactone unit is between about 30% and about 100%, calculated lactone unit versus. (lactone unit+diester/diacid). Expressed in terms of a molar ratio, the lactone unit versus. (lactone unit+diester/diacid) content is between about 0.3 and about 1, i.e., x/(x+q) is between about 0.3 and about 1. Preferably, the number of carbon atoms in the lactone unit is between about 10 and about 24, more preferably the number of carbon atoms in the lactone unit is between about 12 and about 16. Most preferably, the number of carbon atoms in the lactone unit is 12 (dodecalactone), 15 (pentadecalactone), or 16 (hexadecalactone).

The structure of a PACE-PEG-containing polymer is shown below:

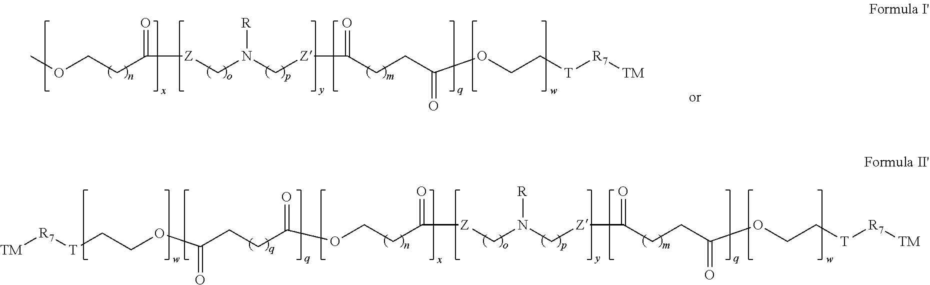

preferably Formula I′,
wherein n is an integer from 1-30, m, o, and p are independently an integer from 1-20, x, y, and q are independently integers from 1-1000, w is independently an integer from 0-1000, with the proviso that in Formula I′, w is greater than 0, and in Formula II′, at least one w is not zero (0),
Z and Z′ are independently O or NR′, wherein R and R′ are independently hydrogen, substituted or unsubstituted alkyl, or substituted or unsubstituted aryl,
wherein T is independently absent, oxygen, sulfur, alkyl, substituted alkyl, amide, substituted amide, amine, substituted amine, carbonyl, substituted carbonyl,
wherein R₇ is independently hydrogen, alkyl, substituted alkyl, amide, substituted amide, substituted sulfone, unsubstituted sulfone, aryl, substituted aryl, cycloalkyl, substituted cycloalkyl, maleimide, amine, substituted amine, thiol, N-hydroxysuccinimide ester, succinimide, azide, acrylate, methacrylate, alkyne, hydroxide, or isocyanate, and
TM is independently absent or a targeting moiety.

In some forms of the PACE-PEG polymer, Z is the same as Z′.

In some forms of the PACE-PEG polymer, Z is O and Z′ is O. In some forms of the PACE-PEG polymer, Z is NR′ and Z′ is NR′. In some forms of the PACE-PEG polymer, Z is O and Z′ is NR′. In some forms of the PACE-PEG polymer, Z is NR′ and Z′ is O.

In some forms of the PACE-PEG polymer, Z′ is O and n is an integer from 1-24, such as 4, 10, 13, or 14. In some forms of the PACE-PEG polymer, Z is also O.

In some forms of the PACE-PEG polymer, Z′ is O, n is an integer from 1-24, such as 4, 10, 13, or 14, and m is an integer from 1-10, such as 4, 5, 6, 7, or 8. In some forms of the PACE-PEG polymer, Z is also O.

In some forms of the PACE-PEG polymer, Z′ is O, n is an integer from 1-24, such as 4, 10, 13, or 14, m is an integer from 1-10, such as 4, 5, 6, 7, or 8, and o and p are the same integer from 1-6, such 2, 3, or 4. In some forms of the PACE-PEG polymer, Z is also O.

In some forms of the PACE-PEG polymer, Z′ is O, n is an integer from 1-24, such as 4, 10, 13, or 14, m is an integer from 1-10, such as 4, 5, 6, 7, or 8, and R is alkyl, such a methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, and n-octyl, or aryl, such as phenyl, naphthalyl, anthracenyl, phenanthryl, chrysenyl, pyrenyl, tolyl, or xylyl. In some forms, Z is also O.

In some forms of the PACE-PEG polymer, n is 14 (e.g., pentadecalactone, PDL), m is 7 (e.g., sebacic acid), o and p are 2 (e.g., N-methyldiethanolamine, MDEA).

In particular embodiments, the values of x, y, q, and w are such that the weight average molecular weight of the PACE-PEG polymer is greater than 5,000 Daltons, such as between 5,000 Daltons and 50 kDa, or between 5,000 Daltons and 30 kDa. This weight average molecular weight does not include the molecular weight of any targeting moiety. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

The blocks of polyalkylene oxide can located at the termini of the polymer (i.e., by reacting PEG having one hydroxy group blocked, for example, with a methoxy group), within the polymer backbone (i.e., neither of the hydroxyl groups are blocked), or combinations thereof.

In some forms, the particles contain PACE-PEG polymers of Formula I′ and/or Formula II′, as described above, wherein at least a targeting moiety (TM) is present in Formula I and/or Formula II′. Preferably, covalent conjugation occurs through T, R₇, or a combination thereof.

In some forms, such as when there is a targeting moiety such as a protein or peptide, conjugation proceeds via a facile “click” reaction between a reactive group on the protein or peptide (e.g., a thiol) and a maleimide group on the polymer to form a succinimide group linking the targeting moiety to the PEG terminal of the polymer.

2. Particles Containing PACE-PAO Blended with a Second PACE Polymer

In some forms, the particles contain a blend of a PACE-PAO polymer (as described above under the section titled Particles containing PACE-PAO not blended with a second PACE polymer) and a second PACE polymer.

PACE polymeric particles, including solid core particles formed therefrom are known. See, for example, U.S. Pat. Nos. 9,272,043, 9,567,430, 10,682,422, 10,465,042, 11,136,597, 10,765,638.

When used to deliver genetic materials, the transfection efficiency of the polymers is strongly dependent on the end groups on the polymers. When substituting the diester monomer in the polymers with diacid, such as sebacic acid, polymers with a mixture of hydroxyl and carboxyl end groups can be obtained. Both of these two end groups can be activated with 1,1′-carbodiimidazole. The activated product can react with amine-containing molecules to yield polymers with new end groups.

The polymers can be further hydrolyzed to release more active end groups, such as —OH and —COOH, both of which can originate from hydrolysis of ester bonds in the polymers (also referred to herein as “actuation”), typically by incubating the polymers, e.g., at a control temperature (e.g., 37° C. or 100° C.), for days or weeks. In some embodiments, the polymers are not hydrolyzed, and thus can be referred to as “non-actuated.”

In some embodiments, the content of a hydrophobic monomer in the polymer is increased relative the content of the same hydrophobic monomer when used to form particles. Increasing the content of a hydrophobic monomer in the polymer forms a polymer that can form solid core nanoparticles in the presence of nucleic acids, including RNAs. Preferred particles contain 10-20% PDL in the PACE polymer. Solid core particles preferably contain 60% PDL.

These particles are stable for long periods of time during incubation in buffered water, or serum, or upon administration (e.g., injection) into animals. They also provide for a sustained release of nucleic acids (e.g., siRNA) which leads to long term activity (e.g., siRNA mediate-knockdown).

Preferably, the second PACE polymer does not contain a PEG modification. The second PACE polymer may be the same or different from the PACE polymer segment in the PACE-PAO (such as PACE-PEG) polymer, where similarities or differences can be assessed based on weight average molecular weights, or molar percent compositions of components in the PACE polymers. The second PACE polymer optionally contains endgroup modifications. When the second PACE polymer does not contain an endgroup modification, it is denoted “second PACEab.” When at least one endgroup modification is present, the second PACE polymer is denoted “second PACEng.”

In some forms, the second PACE polymers have a structure as shown in Formula I:

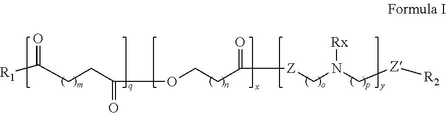

wherein n is an integer from 1-30,
m, o, and p are independently integers from 1-20,
x, y, and q are independently integers from 1-1000,
Rₓ is hydrogen, substituted or unsubstituted alkyl, or substituted or unsubstituted aryl, or substituted or unsubstituted alkoxy,
Z and Z′ are independently O or NR′, wherein R′ is hydrogen, substituted or unsubstituted alkyl, or substituted or unsubstituted aryl,
R₁ and R₂ are independently absent or are chemical entities containing a hydroxyl group, a primary amine group, a secondary amine group, a tertiary amine group, or combinations thereof.

In some forms of Formula I, R₁ and R₂ are absent. In some forms of Formula I, at least one of R₁ and R₂ is present. When R₁ and R₂ are absent, the PACE polymer is denoted “second PACEab polymer.” When at least one of R₁ and R₂ is present, the PACE polymer is denoted “second PACEng polymer.”

Examples of Rₓ and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

In particular embodiments, the values of x, y, and/or q are such that the weight average molecular weight of the second PACE polymer (i.e., second PACEab polymer or second PACEng polymer) is greater than 20,000 Daltons, greater than 15,000 Daltons, greater than 10,000 Daltons, greater than 5,000 Daltons, greater than 2,000 Daltons. In some forms, the weight average molecular weight of the second PACE polymer (i.e., second PACEab polymer or second PACEng polymer) is between about 2,000 Daltons and about 20,000 Daltons, more preferably between about 5,000 Daltons and about 10,000 Daltons.

The second PACE polymer (i.e., second PACEab polymer or second PACEng polymer) can be prepared from one or more lactones, one or more amine-diols (Z and Z′═O), triamines (Z and Z′═NR′), or hydroxy-diamines (Z═O and Z′═NR′, or Z═NR′ and Z′═O) and one or more diacids or diesters. In those embodiments where two or more different lactone, diacid or diester, and/or triamine, amine-diol, or hydroxy-diamine monomers are used, the values of n, o, p, and/or m can be the same or different.

In some forms of the second PACE polymer (i.e., second PACEab polymer or second PACEng polymer), the percent composition of the lactone unit is between about 10% and about 100%, calculated lactone unit vs. (lactone unit+diester/diacid). Expressed in terms of a molar ratio, the lactone unit vs. (lactone unit+diester/diacid) content is between about 0.1 and about 1, i.e., x/(x+q) is between about 0.1 and about 1. In some forms of the second PACE polymer (i.e., second PACEab polymer or second PACEng polymer), the percent composition of the lactone unit is between about 30% and about 100%, calculated lactone unit vs. (lactone unit+diester/diacid). Expressed in terms of a molar ratio, the lactone unit vs. (lactone unit+diester/diacid) content is between about 0.3 and about 1, i.e., x/(x+q) is between about 0.1 and about 1. Preferably, the number of carbon atoms in the lactone unit is between about 10 and about 24, more preferably the number of carbon atoms in the lactone unit is between about 12 and about 16. Most preferably, the number of carbon atoms in the lactone unit is 12 (dodecalactone), 15 (pentadecalactone), or 16 (hexadecalactone).

In some forms of the second PACE polymer (i.e., second PACEab polymer or second PACEng polymer), Z is the same as Z′.

In some forms of the second PACE polymer (i.e., second PACEab polymer or second PACEng polymer), Z is O and Z′ is O. In some forms of the second PACE polymer (i.e., second PACEab polymer or second PACEng polymer), Z is NR′ and Z′ is NR′. In some forms of the second PACE polymer (i.e., second PACEab polymer or second PACEng polymer), Z is O and Z′ is NR′. In some forms, Z is NR′ and Z′ is O.

In some forms of the second PACE polymer (i.e., second PACEab polymer or second PACEng polymer), Z′ is O and n is an integer from 1-24, such as 4, 10, 13, or 14. In some forms of the second PACE polymer (i.e., second PACEab polymer or second PACEng polymer), Z is also O.

In some forms of the second PACE polymer (i.e., second PACEab polymer or second PACEng polymer), Z′ is O, n is an integer from 1-24, such as 4, 10, 13, or 14, and m is an integer from 1-10, such as 4, 5, 6, 7, or 8. In some forms of the second PACE polymer (i.e., second PACEab polymer or second PACEng polymer), Z is also O.

In some forms of the second PACE polymer (i.e., second PACEab polymer or second PACEng polymer), Z′ is O, n is an integer from 1-24, such as 4, 10, 13, or 14, m is an integer from 1-10, such as 4, 5, 6, 7, or 8, and o and p are the same integer from 1-6, such 2, 3, or 4. In some forms, Z is also O.

In some forms of the second PACE polymer (i.e., second PACEab polymer or second PACEng polymer), Z′ is O, n is an integer from 1-24, such as 4, 10, 13, or 14, m is an integer from 1-10, such as 4, 5, 6, 7, or 8, and R is alkyl, such a methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, and n-octyl, or aryl, such as phenyl, naphthalyl, anthracenyl, phenanthryl, chrysenyl, pyrenyl, tolyl, or xylyl. In some forms of the second PACE polymer (i.e., second PACEab polymer or second PACEng polymer), Z is also O.

In some forms of the second PACE polymer (i.e., second PACEab polymer or second PACEng polymer), n is 14 (e.g., pentadecalactone, PDL), m is 7 (e.g., sebacic acid), o and p are 2 (e.g., N-methyldiethanolamine, MDEA).

In some forms, the particles contain the second PACEng polymer, wherein R₁ and/or R₂ are not relative to corresponding particles wherein R₁ and/or R₂ consist of or include

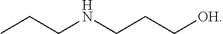

In some embodiments, particles formed from the polymer show improved loading, improved cellular transfection, improved intracellular endosomal release, or a combination thereof of a nucleic acid cargo, such as RNA, more particularly mRNA, relative to corresponding particles wherein R₁ and/or R₂ consist of or include

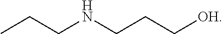

In some forms, the second PACEng polymer has a structure of Formula II.

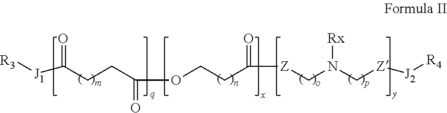

wherein J₁ and J₂ are independently linking moieties or absent,
R₃ and R₄ are independently substituted alkyl containing a hydroxyl group, a primary amine group, a secondary amine group, a tertiary amine group, or combinations thereof. In some forms, the molecular weight of R₃, R₄ or both are at or below 500 Daltons, at or below 200 Daltons, or at or below 100 Daltons.

In some forms of the second PACEng polymer, J₁ is —O— or —NH—.

In some forms of the second PACEng polymer, J₂ is —C(O)NH— or —C(O)O—.

In some forms of the second PACEng polymer, R₃ is identical to R₄.

Preferably, R₃ and/or R₄ are linear.

In some forms of the second PACEng polymer, R₃, R₄ or both contain a primary amine group. In some forms, R₃, R₄ or both contain a primary amine group and one or more secondary or tertiary amine groups.

In some forms of the second PACEng polymer, R₃, R₄ or both contain a hydroxyl group. In some forms of the second PACEng polymer, R₃, R₄ or both contain a hydroxyl group and one or more amine groups, preferably secondary or tertiary amine groups. In some forms of the second PACEng polymer, R₃, R₄ or both contain a hydroxyl group and no amine group.

In some forms of the second PACEng polymer, at least one of R₃ and R₄ does not contain a hydroxyl group.

In some forms of the second PACEng polymer, R₃, R₄ or both are -unsubstituted C₁-C₁₀ alkylene-Aq-unsubstituted C₁-C₁₀ alkylene-Bq, -unsubstituted C₁-C₁₀ alkylene-Aq-substituted C₁-C₁₀ alkylene-Bq, -substituted C₁-C₁₀ alkylene-Aq-unsubstituted C₁-C₁₀ alkylene-Bq, or -substituted C₁-C₁₀ alkylene-Aq-substituted C₁-C₁₀ alkylene-Bq, wherein Aq is absent or —NR₅—, and Bq is hydroxyl, primary amine, secondary amine, or tertiary amine, wherein R₅ is hydrogen, substituted or unsubstituted alkyl, or substituted or unsubstituted aryl.

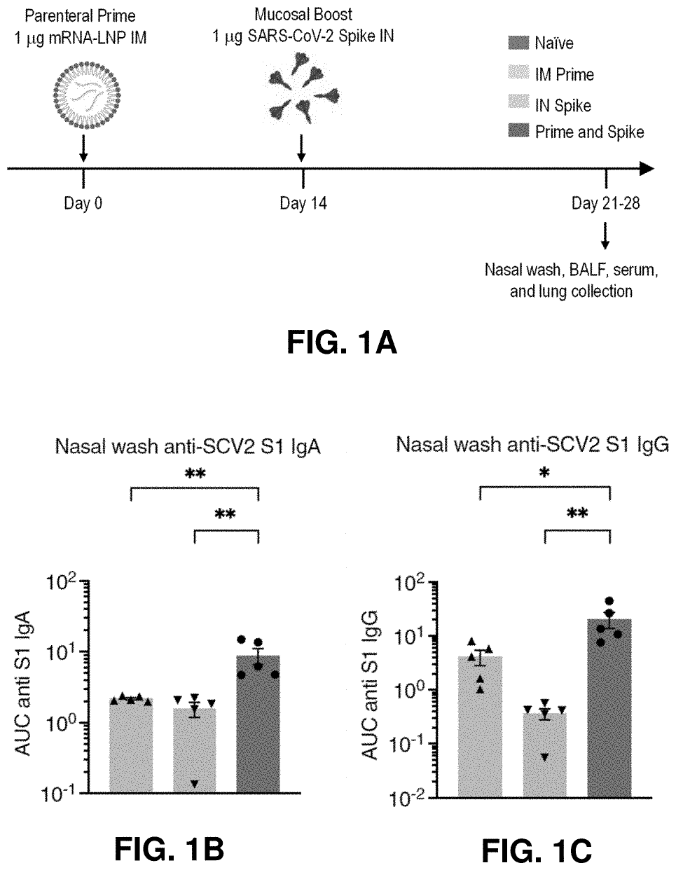

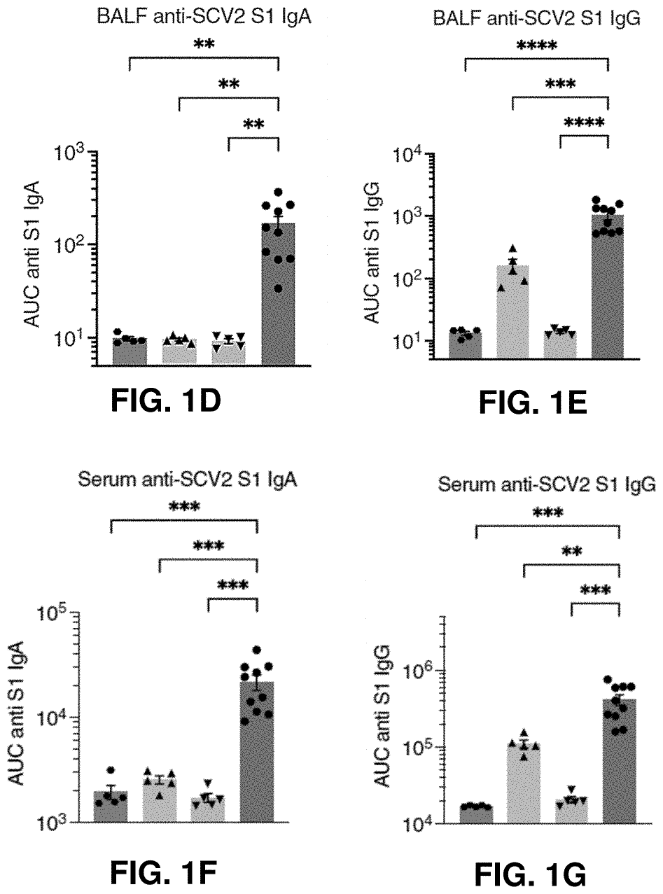

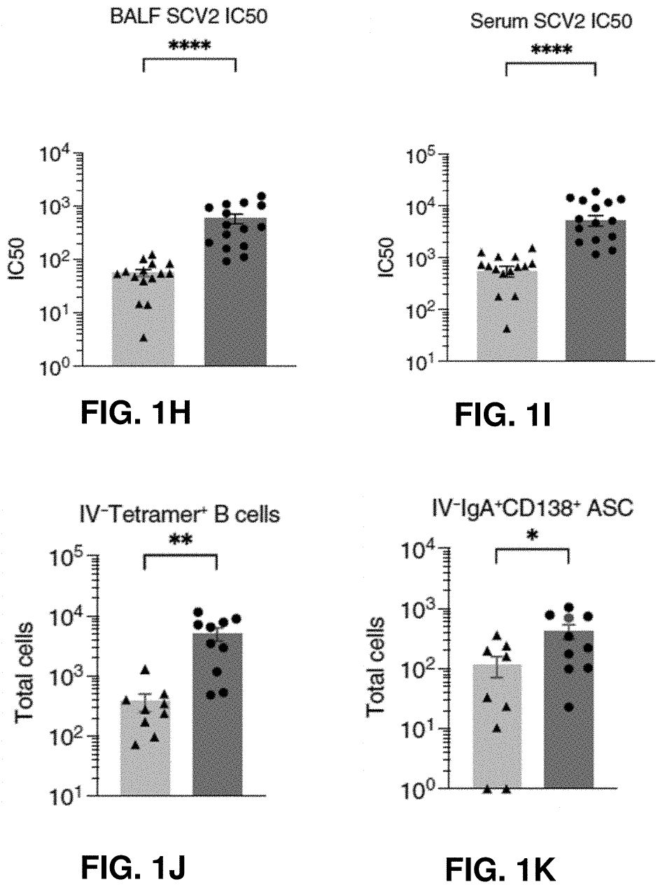

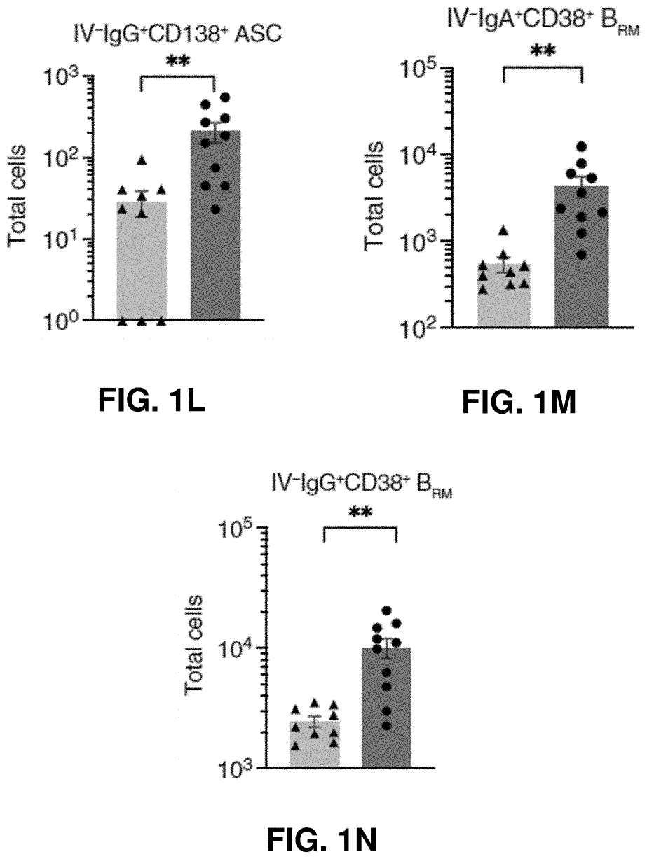

In some forms of the second PACEng polymer, R₃, R₄ or both are selected from the groups shown in FIG. 1.

In some forms, the second PACEng polymer is as described above, except that the second PACEng polymer has a structure of Formula III.

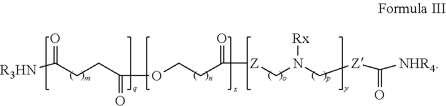

The monomer units can be substituted at one or more positions with one or more substituents. Exemplary substituents include, but are not limited to, alkyl groups, cyclic alkyl groups, alkene groups, cyclic alkene groups, alkynes, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The second PACE polymer (i.e., second PACEab polymer or second PACEng polymer) is biocompatible. Readily available lactones of various ring sizes are known to possess low toxicity: for example, polyesters prepared from small lactones, such as poly(caprolactone) and poly(p-dioxanone) are commercially available biomaterials which have been used in clinical applications. Large (e.g., C₁₆-C₂₄) lactones and their polyester derivatives are natural products that have been identified in living organisms, such as bees. Lactones containing ring carbon atoms between 16 and 24 are specifically contemplated and disclosed.

In some forms, the second PACE polymers (i.e., second PACEab polymer or second PACEng polymer) can be further activated via temperature-controlled hydrolysis, thereby exposing one or more activated end group(s). The one or more activated end group(s) can be, for example, hydroxyl or carboxylic acid end groups, both of which can be generated via hydrolysis of ester bonds within the polymers. The activated second PACE polymers (i.e., second PACEab polymer or second PACEng polymer) can have a weight-average molecular weight between about 5 and 25 kDa, preferably between about 5 and 10 kDa. As used herein, the term “about” is meant to minor variations within acceptable parameters. For the sake of clarity, “about” refers to ±10% of a given value. In some forms, the activated second PACE polymers contain R₁ or R₂ (such as chemical entities containing a hydroxyl group, a primary amine group, a secondary amine group, a tertiary amine group, or combinations thereof, exemplified by the non-limiting moieties denoted R₃ or R₄) at one end, and a hydroxyl or carboxylic acid end group at the other end, generated via hydrolysis.

In some forms, the second PACEng polymer has a structure of Formula IV.

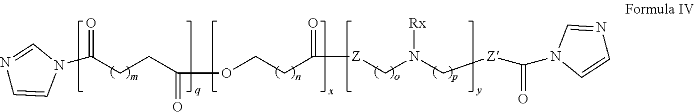

In some forms, the second PACEng polymer has a structure of Formula V.

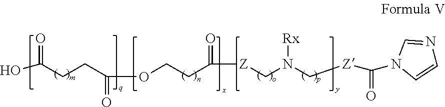

In some forms, the second PACEng polymer has a structure of Formula VI.

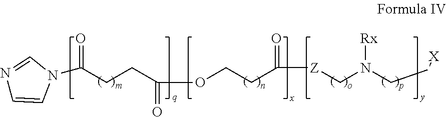

wherein X′ is —OH or —NHR′.

Formulas VI, V, and VI are structures of intermediary products. They can be used to synthesize a wide variety of second PACEng polymers with a structure of Formula I, II or III, wherein at least one of R₁ and R₂ is present.

3. Particles Containing Non-Covalently Conjugated Surfactant on the Surface

In another aspect, the particles contain a surfactant non-covalently conjugated to the surface of the particles. In these forms, the particles contain a second PACE polymer, as described above under the section titled Particles containing PACE-PAO blended with a second PACE polymer. Preferably, the surfactant is a polymeric surfactant.

Preferably in these forms, the surface of the particles contains a surfactant to which a targeting moiety is covalently conjugated. The surfactant, containing the targeting moiety, contains a structure:

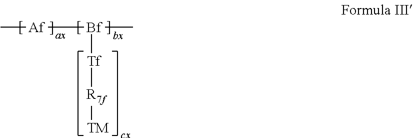

wherein:
each Af is independently a hydrophobic or hydrophilic monomer residue,
Bf is substituted alkyl, substituted aryl, substituted cycloalkyl, substituted heterocyclyl, substituted amine, substituted carbonyl, substituted amide, or substituted sulfone,
wherein Tf is independently oxygen or is absent,
wherein R_{7f} is independently hydrogen, alkyl, substituted alkyl, amide, substituted amide, substituted sulfone, unsubstituted sulfone, aryl, substituted alkyl, cycloalkyl, substituted cycloalkyl, maleimide, amine, substituted amine, thiol, N-hydroxysuccinimide ester, succinimide, azide, acrylate, methacrylate, alkyne, hydroxide, or isocyanate,
TM is a targeting moiety,
ax is independently an integer between 0 and 10,000, between 0 and 5,000, or between 0 and 1,000, and
bx and cx are independently an integer between 1 and 10,000, between 1 and 5000, or between 1 and 1,000.

In some forms, the surfactant containing the targeting moiety is as described above, except that the surfactant contains a structure:

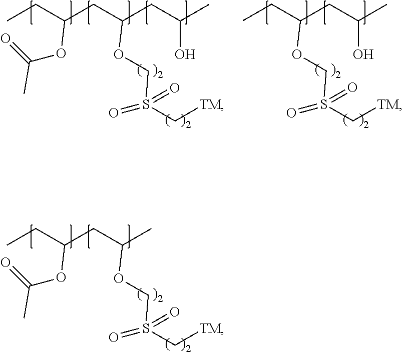

In either approach to conjugating targeting moieties to the nanoparticles, targeting moieties include small molecules (molecular weight between 200 Da and 2,500 Da), aptamers, proteins, or peptides.

Poly(amine-co-ester)s or poly(amine-co-amide)s with improved properties for delivery, including delivery to the lungs have been developed.

In some forms, these PACE polymers are modified with a polyalkylene oxide. These PACE-PAO polymers contain PACE-PAO polymer structures, as described above under the section titled Particles containing PACE-PAO not blended with a second PACE polymer.

In some forms, the PACE polymers are not modified with a polyalkylene oxide. In these forms, the PACE polymers contain PACE polymer structures akin to the second PACE polymers, as described above under the section titled Particles containing PACE-PAO blended with a second PACE polymer.

In some forms, the PACE polymers contain the structure,

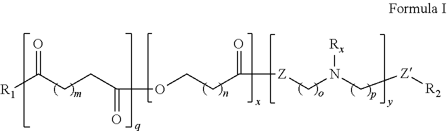

wherein n is an integer from 1-30,
m, o, and p are independently integers from 1-20,
x, y, and q are independently integers from 1-1000,
Rₓ is hydrogen, substituted or unsubstituted alkyl, or substituted or unsubstituted aryl, or substituted or unsubstituted alkoxy,
Z and Z′ are independently O or NR′, wherein R′ is hydrogen, substituted or unsubstituted alkyl, or substituted or unsubstituted aryl,
R₁ and R₂ are chemical entities containing a hydroxyl group, a primary amine group, a secondary amine group, a tertiary amine group, or combinations thereof.

In some forms, Z is the same as Z′.

In some forms, Z is O and Z′ is O. In some forms, Z is NR′ and Z′ is NR′. In some forms, Z is O and Z′ is NR′. In some forms, Z is NR′ and Z′ is O.

In some forms, Z′ is O and n is an integer from 1-24, such as 4, 10, 13, or 14. In some forms, Z is also O.

In some forms, Z′ is O, n is an integer from 1-24, such as 4, 10, 13, or 14, and m is an integer from 1-10, such as 4, 5, 6, 7, or 8. In some forms, Z is also O.

In some forms, Z′ is O, n is an integer from 1-24, such as 4, 10, 13, or 14, m is an integer from 1-10, such as 4, 5, 6, 7, or 8, and o and p are the same integer from 1-6, such 2, 3, or 4. In some forms, Z is also O.

In some embodiments, Z′ is O, n is an integer from 1-24, such as 4, 10, 13, or 14, m is an integer from 1-10, such as 4, 5, 6, 7, or 8, and R is alkyl, such a methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, and n-octyl, or aryl, such as phenyl, naphthalyl, anthracenyl, phenanthryl, chrysenyl, pyrenyl, tolyl, or xylyl. In some forms, Z is also O.

In some forms, n is 14 (e.g., pentadecalactone, PDL), m is 7 (e.g., sebacic acid), o and p are 2 (e.g., N-methyldiethanolamine, MDEA).

In some forms, the polymer contains a structure of Formula II:

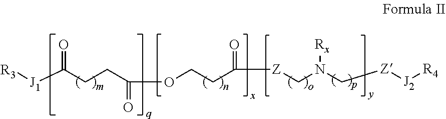

wherein Rx, Z, Z′, m, n, o, p, q, x, and y are as described above for Formula I,
J₁ and J₂ are independently absent or linking moieties such as —C(O)—, —C(O)NH—, —C(O)O—, —O—, and —NH—,
R₃ and R₄ in Formula II are independently substituted alkyl containing a hydroxyl group, a primary amine group, a secondary amine group, a tertiary amine group, or combinations thereof.

In some forms, J₁ is —O— or —NH—.

In some forms, J₂ is —C(O)—, —C(O)NH—, or —C(O)O—.

In some forms, R₃ is identical to R₄.

Preferably, R₃ and/or R₄ are linear.

In some forms, R₃, R₄, or both contain a hydroxyl group. In some forms, R₃, R₄, or both contain a hydroxyl group and one or more amine groups, preferably secondary or tertiary amine groups. In some forms, R₃, R₄, or both contain a hydroxyl group and no amine group.

In some forms, at least one of R₃ and R₄ does not contain a hydroxyl group.

In some forms, the polymer contains a structure of Formula III:

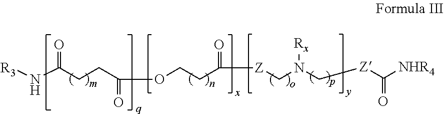

wherein R₃, R₄, Rx, Z, Z′, m, n, o, p, q, x, and y are as described above for Formula II.

In particular embodiments, the values of x, y, and/or q are such that the weight average molecular weight of the polymer is greater than 20,000 Daltons, greater than 15,000 Daltons, greater than 10,000 Daltons, greater than 5,000 Daltons, greater than 2,000 Daltons. In some forms, the weight average molecular weight of the polymer is between about 2,000 Daltons and about 20,000 Daltons, more preferably between about 5,000 Daltons and about 10,000 Daltons.

The polymer can be prepared from one or more lactones, one or more amine-diols (Z and Z′═O) or triamines (Z and Z′═NR′), and one or more diacids or diesters. In those embodiments where two or more different lactone, diacid or diester, and/or triamine or amine-diol monomers are used, the values of n, o, p, and/or m can be the same or different.

In some embodiments, the polymers are between about 2 kDa and 20 kDa, or between about 2 kDa and about 10 kDa, or between about 2 kDa and about 5 kDa.

The poly(amine-co-ester)s or poly(amine-co-amide)s can be used to form particles, such as microparticles, and/or nanoparticles preferably having encapsulated therein one or more messenger ribonucleic acids. In some forms, the particles are formed from polymer wherein R₁ and/or R₂ do not consist of or include

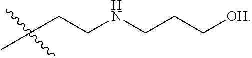 [SMILES: *CCNCCCO]

In some forms, the particles contain surfactants non-covalently conjugated to the surface of the particles. The structure of the surfactants can be as described above under the section titled Particles containing non-covalently conjugates surfactant on the surface.

In some forms, the poly(amine-co-ester)s or poly(amine-co-amide)s are in a mixture containing the poly(amine-co-ester)s or poly(amine-co-amide)s conjugated to a polyethylene glycol (PEG), i.e., PEG-conjugated poly(amine-co-ester)s or poly(amine-co-amide)s.

In some forms, the PEG-conjugated poly(amine-co-ester)s or poly(amine-co-amide)s contain a structure

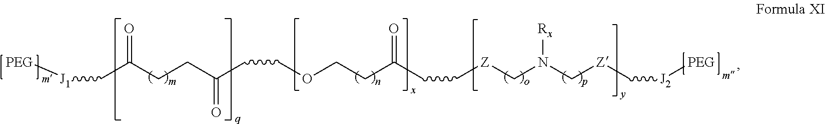

wherein m′ and m″ are independently 0 or 1, with the proviso that m′+m″ is 1 or 2,
J₁ and J₂ in Formulae XI are independently absent or linking moieties such as —C(O)—, —C(O)NH—, —C(O)O—, —O—, and —NH—.

In some forms of Formula XI, J₁ is —O— or —NH—. In some forms of Formula XI, J₂ is —C(O)—, —C(O)NH—, or —C(O)O—.

4. Sources of Polymers

The polymers are generally modified from synthetic polymers. Exemplary synthetic polymers include poly(amine-co-ester), formed of a lactone, a dialkyl acid, and a dialkyl amine. Methods for the synthesis of poly(amine-co-ester) from a lactone, a dialkyl acid, and a dialkyl amine using an enzyme catalyst, such as a lipase, are also provided. Exemplary lactones are disclosed in U.S. Patent Publication No. US20170121454. In some forms, poly(amine-co-ester) is prepared as shown in Scheme 1:

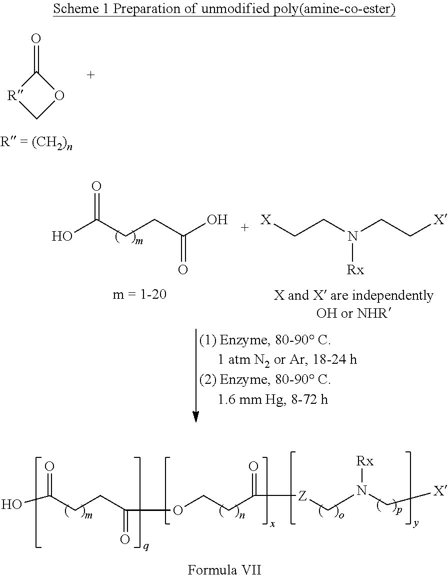

The molar ratio of the monomers (e.g., lactone:aminodiol:diacid) can vary, for example from about 10:90:90 to about 90:10:10. In some embodiments, the ratio is 10:90:90, 20:80:80, 40:60:60, 60:40:40, or 80:20:20. The weight average molecular weight, as determined by GPC using narrow polydispersity polystyrene standards, can vary for example from about 2,000 Daltons to about 50,000 Daltons, preferably from about 2,000 Daltons to about 20,000 Daltons, more preferably from about 5000 Daltons to about 20,000 Daltons, most preferably from about 5000 Daltons to about 10,000 Daltons.

The hydrophobicity of the polymers can be adjusted by varying the percentages of lactone, such as between about 10% and about 100% (calculated lactone unit vs. (lactone unit+diester/diacid)). The molecular weight of the polymers can be adjusted by tuning the second stage reaction time, such as between about 8 and about 72 h.

The enzymatic method allows for the synthesis of polymers with diverse chain structures and tunable hydrophobicities. In some embodiments, the hydrophobicity is varied by varying the ring size and/or molar amount of the lactone monomer. Lactone with a wide range of ring sizes (e.g., C₄-C₂₄, preferably C₆-C₂₄, more preferably from C₆-C₁₆) can be used as comonomers. The reaction can be performed in a single step without protection and deprotection of the amino group(s). Such amino-bearing copolyesters are extremely difficult to prepare using conventional organometallic catalysts, as such catalysts are often sensitive to or deactivated by organic amines. These catalysts are also known to be inefficient for polymerizing large lactone ring monomers. Enzymatic catalysts have distinct advantages for producing biomedical polymers owing to the high activity and selectivity of the enzyme and the resulting high purity of products that are metal-free.

Polymers with a structure of Formula IV, V, or VI can be synthesized via reacting the unmodified polymer of Formula VII with 1,1′-carbonyldiimidazole (CDI), at a molar ratio from about 1:10 to about 1:60, preferably at about 1:40.

Polymers with a structure of Formulas I or II can be obtained via modification of the end groups of the unmodified polymer of Formula VII using coupling reactions known in the art. For example, polymers with a structure of Formula III can be synthesized via (1) reacting the unmodified polymer of Formula VII with CDI to obtain a polymer of Formula IV, and (2) reacting the polymer of Formula IV with R₃—NH₂ and R₄—NH₂. In some forms, R₃, R₄, or both are selected from those shown in FIG. 1. Preferably, R₃ and R₄ are the same.

Alternatively, polymers with a structure of Formula III can be synthesized via (1) reacting the unmodified polymer of Formula VII with CDI to obtain a polymer of Formula V or VI, (2) protecting the —COOH group or the —X′ group in the polymer from step (1), (3) reacting the protected polymer from step (2) with R₄—NH₂ or R₃—NH₂, (4) deprotecting the —COOH group or the —X′ group in the polymer from step (3), and (5) reacting the deprotected polymer from step (4) with R₃—NH₂ or R₄—NH₂.

Hydrolysis-mediated activation of the polymers of Formula I, II, or III can be performed in a temperature-controlled manner for up to 30 days or more. The length of hydrolysis may vary depending on the molecular weight of the polymers to be activated. Larger molecular weight polymers (e.g., about 20-25 kDa) are optimally hydrolyzed for longer periods of time, for example, for about 30 to 40 days. Smaller molecular weight polymers (e.g., about 5-7 kDa) are optimally hydrolyzed for shorter periods of time, for example, for about 4 to 10 days.

In some forms, the polymers are hydrolyzed at a temperature from about 30° C. to 42° C., or any in the range of up to about 100° C. The PACE polymers can be hydrolyzed at a temperature from about 35° C. to 40° C., e.g., about 37° C.

In some forms, the polymers are hydrolyzed, for example, at about 1 atm. Higher pressures accelerate the process (e.g., pressures from about 1 to about 100 atm). The rate for the process would be determined by one of skill in the art for the specific formulations being made.

The weight-average molecular weight of the resulting hydrolysis product can vary from about 5 kDa to about 25 kDa, preferably between about 5 and about 10 kDa.

Preferably, one or more of the ester bonds in the polymers are hydrolyzed. The hydrolysis product can have R₁ or R₂ at one end and a carboxyl or a hydroxyl group at the other end, generated via hydrolysis.

The PEG-conjugated poly(amine-co-ester) or poly(amine-co-amide) can be synthesized under conditions similar to those described in Scheme 1, except that in addition to (i) the lactone, (ii) the diacid/diester, and (iii) amine diol and/or triamine, PEG containing a terminal carboxyl, hydroxyl, or amine group can be added to the reactants.

5. Micelles Formed from the Polymers

The polymers, such as PEG-block containing polymers, can be used to prepare micelles. The average micelle size is typically in the range from about 100 to about 500 nm, preferably from about 100 to about 400 nm, more preferably from about 100 to about 300 nm, more preferably from about 150 to about 200 nm, most preferably from about 160 to about 190 nm, which were stable at physiological pH of 7.4 in the presence of serum proteins. The copolymers possess high blood compatibility and exhibit minimal activity to induce hemolysis and agglutination.

The size and zeta potential of the micelles were found to change significantly when the pH of the aqueous medium accommodating the micelles was varied. For example, the trends in the size-pH and zeta-pH curves are remarkably similar for the micelles of the three PEG2K-PPMS copolymers with different PDL contents (11%, 30%, and 51%). It is evident that the average size of the micelle samples gradually increases upon decreasing the medium pH from 7.4 to 5.0, and then remains nearly constant when the pH value is below 5.0. This pH-responsive behavior observed for the micelles is expected upon decreasing the pH from 7.4 to 5.0, the PPMS cores of the micelles become protonated and more hydrophilic, thus absorbing more water molecules from the aqueous medium to cause swelling of the micelles. The micelle cores are already fully protonated at pH of 5.0, and as a result, the sizes of the micelles remain fairly constant with further decreasing of the pH from 5.0. The effects of the PDL content in the PEG2K-PPMS copolymers on the magnitude of the micelle size change between 7.4 and 5.0 pH values are also notable. With decreasing PDL content and increasing tertiary amino group content in the copolymer, the capacity of the micelle cores to absorb protons and water molecules is expected to increase. Thus, upon decreasing pH from 7.4 to 5.0, the change in average micelle size was more significant for PEG2K-PPMS-11% PDL (from 200 nm to 234 nm) as compared to PEG2K-PPMS-30% PDL (from 184 nm to 214 nm) and PEG2K-PPMS-51% PDL (from 163 nm to 182 nm).

The zeta potential of the micelles in aqueous medium also exhibits substantial pH-dependence. At physiological and alkaline pH (7.4 to 8.5), the surface charges of blank PEG2K-PPMS copolymer micelles were negative, which changed to positive when the pH of the medium decreased to acidic range (4.0-6.0). For example, the micelles of PEG2K-PPMS-11% PDL, PEG2K-PPMS-30% PDL, and PEG2K-PPMS-51% PDL possessed zeta potential values of −5.8, −7.1, −5.1 mV, respectively, at pH of 7.4, which turned to +7.6, +5.8, +4.0 mV, correspondingly, at a lower pH of 5.0. On the basis of the above discussions, this surface charge dependence on pH is attributable to the protonation or deprotonation of the PPMS cores of the micelles at different medium pH. At an alkaline pH (7.4-8.5), most of the amino groups in the micelles presumably are not protonated, and the micelle particles remain negatively charged due to the absorption of HPO₄₂— and/or H₂PO₄— anions in PBS by the micelles. In particular, at pH of 8.5, the zeta-potential values were −8.1 mV, −7.9 mV, −9.0 mV for PEG2K-PPMS-11% PDL, PEG2K-PPMS-30% PDL, and PEG2K-PPMS-51% PDL, respectively. Upon decreasing pH from 7.4 to 5.0, the tertiary amino moieties in the micelle PPMS cores become mostly protonated, turning the micelles to positively charged particles. Consistently, among the three micelle samples, PEG2K-PPMS-11% PDL micelles with the largest capacity to absorb protons displayed the highest zeta potential values at pH of 4.0-5.0, whereas PEG2K-PPMS-51% PDL micelles with the smallest protonation capacity showed the lowest zeta potentials. The observed micelle surface charge responses to the medium pH are highly desirable since the negative surface charge of the micelles at physiological pH can alleviate the interaction of the micelles with serum protein in the blood and prolong their in vivo circulation time. On the other hand, the reverse to positive surface charge at the tumor extracellular pH of approximately 6.5 could enhance the uptake of these micelles by target tumor cells.

The surface charge of the particles/micelles were slightly negative in PBS solution (0.01M, pH=7.4), which are beneficial for in vivo drug delivery applications of the micelles. It is known that nanoparticles with nearly neutral surface charge (zeta potential between −10 and +10 mV) can decrease their uptake by the reticuloendothelial system (RES) and prolong their circulation time in the blood. The negative surface charges of the micelles could result from the absorption of HPO₄²⁻ and/or H₂PO₄⁻ anions in PBS by the micelle particles via hydrogen bonding interactions between the anions and the ether groups of PEG shells or the amino groups of PPMS cores. For amphiphilic block copolymer micelles, it is anticipated that hydrophilic chain segments (e.g., PEG) in the outer shell of the micelles can shield the charges in the micelle core with the long chain blocks being more effective in reducing zeta potential than the short chain blocks. Thus, significantly lower zeta potential values were observed for PEG5K-PPMS copolymer micelles as compared to PEG2K-PPMS copolymer micelles.

The copolymer micelles are pH-responsive: decreasing the medium pH from 7.4 to 5.0, the sizes of the micelles significantly increased micelle size while the micelle surface charges reversed from negative charges to positive charges. Correspondingly, DTX-encapsulated copolymer micelles showed gradual sustained drug release at pH of 7.4, but remarkably accelerated DTX release at acidic pH of 5.0. This phenomenon can be exploited to improve release of agents at tumor site, since it is known that the tumor microenvironment is typically weakly acidic (e.g., 5.7-7.0) as the result of lactic acid accumulation due to poor oxygen perfusion. In contrast, the extracellular pH of the normal tissue and blood is slightly basic (pH of 7.2-7.4). Thus, enhanced drug delivery efficiency is anticipated for anticancer drug-loaded micelles that are pH-responsive and can be triggered by acidic pH to accelerate the drug release. Furthermore, even more acidic conditions (pH=4.0-6.0) are encountered in endosomes and lysosomes after uptake of the micelles by tumor cells via endocytosis pathways, which may further increase the cytotoxicity of the drug-encapsulated micelles.

6. Polymeric Microparticles

Typically, the polymers and antigen components are combined into polymeric micro- and/or nanoparticles, having encapsulated therein the one or more antigens, and optionally one or more additional active agents. The antigen can be encapsulated within the particle, dispersed within the polymer matrix that forms the particle, covalently or non-covalently associated with the surface of the particle or combinations thereof. Generally, the antigen is in the form of a nucleic acid.

Typically, the polymer is biocompatible and biodegradable. The nucleic acid(s) encapsulated by and/or associated with the particles can be released through different mechanisms, including diffusion and degradation of the polymeric matrix. The rate of release can be controlled by varying the monomer composition of the polymer and thus the rate of degradation. For example, if simple hydrolysis is the primary mechanism of degradation, increasing the hydrophobicity of the polymer may slow the rate of degradation and therefore increase the time period of release. In all case, the polymer composition is selected such that an effective amount of nucleic acid(s) is released to achieve the desired purpose/outcome.

7. Controlled Release Materials

In some embodiments the particles provide controlled release of active agents encapsulated or otherwise associated with the particles. For example, the unaltered particles may provide release of an effective amount of the drug over time based on the rate of diffusion of the drug form the particle and/or the rate of degradation of the polymer. The polymer composition can be varied to manipulate the degradation behavior of the polymer and thus the release rate/time of the agent to be delivered. Alternatively, the particle can be coated with one or more materials to provide controlled release, such as sustained release or delayed release of the agent or agents to be delivered.

Sustained release and delayed release materials are well known in the art. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

Controlled release polymers known in the art include acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is included of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the EUDRAGIT®. In further preferred embodiments, the acrylic polymer includes a mixture of two acrylic resin lacquers commercially available from Rohm Pharma under the EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multi-particulate systems formed to include the same are swellable and permeable in aqueous solutions and digestive fluids.

The polymers such as EUDRAGIT® RL/RS may be mixed together in any desired ratio in order to ultimately obtain a sustained-release formulation having a desirable dissolution profile. Desirable sustained-release multi-particulate systems may be obtained, for instance, from 100% EUDRAGIT® RL, 50% EUDRAGIT® RL and 50% EUDRAGIT® RS, AND 10% EUDRAGIT® RL and 90% EUDRAGIT® RS. One skilled in the art will recognize that other acrylic polymers may also be used, such as, for example, EUDRAGIT® L.

Other controlled release materials include methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGIT® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac.

B. Vaccine Viral Antigens

A virion consists of a nucleic-acid core, an outer protein coating, and sometimes an outer envelope made of protein and phospholipid membranes derived from the host cell. The most visible difference between members of viral families is their morphology, which is quite diverse. An interesting feature of viral complexity is that the complexity of the host does not correlate to the complexity of the virion. Some of the most complex virion structures are observed in bacteriophages, viruses that infect the simplest living organisms, bacteria.

Viruses come in many shapes and sizes, but these are consistent and distinct for each viral family. All virions have a nucleic-acid genome covered by a protective layer of protein, called a capsid. The capsid is made of protein subunits called capsomeres. Some viral capsids are simple polyhedral “spheres,” whereas others are quite complex in structure. The outer structure surrounding the capsid of some viruses is called the viral envelope. All viruses use some sort of glycoprotein to attach to their host cells at molecules on the cell called viral receptors. The virus exploits these cell-surface molecules, which the cell uses for some other purpose, as a way to recognize and infect specific cell types. For example, the measles virus uses a cell-surface glycoprotein in humans that normally functions in immune reactions and possibly in the sperm-egg interaction at fertilization. Attachment is a requirement for viruses to later penetrate the cell membrane, inject the viral genome, and complete their replication inside the cell.

By stimulating the immune system to interfere with specific antigen, one can inhibit infection (typically through immunization with a viral envelope or capsid protein, preventing infection of the cell, or limiting the spread and infection of new cells following lysis, budding or other release from infected cells.

The polymers described above form solid core particles or micelles or particles formed of or including the polymers, which are useful for delivery of one or more antigens derived from a pathogen to target cells for the purposes of stimulating or inducing protective immunity against infection by the pathogen in the host. Therefore, in preferred embodiments, the polymer compositions deliver one or more exogenous antigens to target cells. In some embodiments, the polymers are used to encapsulate, be mixed with, or be ionically or covalently coupled to any of a variety of antigenic or immunogenic molecules.

It has been established that administration of the polymers, in combination with one or more exogenous antigens, to a mucosal tissue induces a mucosal immune response and/or a long lasting and protective mucosal immunity specific to the antigen in a subject. Typically, compositions for inducing tissue-resident mucosal adaptive immunity include poly(amine-co-ester) polymers and an antigenic or immunogenic protein fragment or epitope. In some embodiments, antigen is a whole protein, an immunogenic peptide fraction of the protein, or a nucleic acid such as DNA or RNA encoding the protein or peptide. In preferred embodiments, the antigen is an mRNA encoding the protein or peptide. Exemplary antigens include bacterial antigens, viral antigens, or tumor antigens.

Antigens can be or can include, for example, proteins, nucleic acids, lipids, and polysaccharides. Exemplary antigens include B cell antigens and T cell antigens. B cell antigens can be peptides, proteins, polysaccharides, saccharides, lipids, nucleic acids, small molecules (alone or with a hapten) or combinations thereof. In a preferred embodiment, the antigen is a polypeptide, or a nucleic acid encoding a polypeptide. The antigen can be derived from a virus, bacterium, parasite, plant, protozoan, fungus, tissue or transformed cell such as a cancer (such as a leukemic cell) and immunogenic component thereof, e.g., cell wall components or molecular components thereof. Thus, the antigen can be a tumor specific antigen) The antigens can be allergens or environmental antigens or tumor antigens. The antigen can be associated with one or more diseases or conditions such as infectious diseases, and autoimmune diseases.

Suitable antigens are known in the art and are available from commercial government and scientific sources. The antigens can be purified, or partially purified polypeptides derived from tumors or viral or bacterial sources. The antigens can be recombinant polypeptides produced by expressing DNA or mRNA encoding the polypeptide antigen in a heterologous expression system. Antigens can be provided as single antigens or can be provided in combination. Antigens can also be provided as complex mixtures of polypeptides or nucleic acids.

The particles may also include antigens and/or adjuvants (i.e., molecules enhancing an immune response). Peptide, protein, and DNA based vaccines may be used to induce immunity to various diseases or conditions. Cell-mediated immunity is needed to detect and destroy virus-infected cells. Most traditional vaccines (e.g., protein-based vaccines) can only induce humoral immunity. DNA-based vaccine represents a unique means to vaccinate against a virus or parasite because a DNA based vaccine can induce both humoral and cell-mediated immunity. In addition, DNA based vaccines are potentially safer than traditional vaccines. DNA vaccines are relatively more stable and more cost-effective for manufacturing and storage. DNA vaccines include two major components: DNA carriers (or delivery vehicles) and DNAs encoding antigens. DNA carriers protect DNA from degradation, and can facilitate DNA entry to specific tissues or cells and expression at an efficient level.

In preferred embodiments, the antigen is in the form of a nucleic acid, for example, a nucleic acid encoding a polypeptide antigen or a portion thereof. Therefore, in some embodiments, the polymers include nucleic acids for delivery into one or more cells and expression of one or more protein antigen in vivo. Accordingly, particles including polymers and one or more polynucleotides are also disclosed. The polynucleotide can encode one or more proteins, functional nucleic acids, or combinations thereof. The polynucleotide can be monocistronic or polycistronic. In some embodiments, polynucleotide is multigenic. For example, the polypeptide encoded by the polynucleotide can be a polypeptide antigen that provides a prophylactic effect to an organism. For example, for immunization against infection such as parasitic, viral, bacterial, fungal or other infections, the polynucleotide(s) to be expressed may encode antigen that stimulates the immune system to provide immunity against the pathogen, such as a respiratory pathogen to stimulate the immune system of an organism

In some embodiments, the polynucleotide is transfected into the cell and remains extrachromosomal. In some embodiments, the polynucleotide is introduced into a host cell and is integrated into the host cell's genome. As discussed in more detail below, the compositions can be used in methods of gene therapy. Methods of gene therapy can include the introduction into the cell of a polynucleotide that alters the genotype of the cell. Introduction of the polynucleotide can correct, replace, or otherwise alter the endogenous gene via genetic recombination. Methods can include introduction of an entire replacement copy of a defective gene, a heterologous gene, or a small nucleic acid molecule such as an oligonucleotide. For example, a corrective gene can be introduced into a non-specific location within the host's genome.

In some embodiments, the polynucleotide is incorporated into or part of a vector. Methods to construct expression vectors containing genetic sequences and appropriate transcriptional and translational control elements are well known in the art. These methods include in vitro recombinant DNA techniques, synthetic techniques, and in vivo genetic recombination. Expression vectors generally contain regulatory sequences and necessary elements for the translation and/or transcription of the inserted coding sequence, which can be, for example, the polynucleotide of interest. The coding sequence can be operably linked to a promoter and/or enhancer to help control the expression of the desired gene product. Promoters used in biotechnology are of different types according to the intended type of control of gene expression. They can be generally divided into constitutive promoters, tissue-specific or development-stage-specific promoters, inducible promoters, and synthetic promoters.

For example, in some embodiments, the antigen nucleic acid is operably linked to a promoter or other regulatory elements known in the art. Thus, the polynucleotide can be a vector such as an expression vector. The engineering of polynucleotides for expression in a prokaryotic or eukaryotic system may be performed by techniques generally known to those of skill in recombinant expression. An expression vector typically includes one of the compositions under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors. It will be appreciated that any of these vectors may be packaged and delivered using the polymers.

Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the compositions. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

In preferred embodiments, the polynucleotide cargo is an RNA, such as an mRNA. The mRNA can encode a polypeptide of interest.

In some embodiments, the mRNA has a cap on the 5′ end and/or a 3′ poly(A) tail which can modulate ribosome binding, initiation of translation and stability mRNA in the cell.

In some embodiments, the antigen is in the form of a polypeptide, for example, a protein antigen. Therefore, in some embodiments, the polymers include polypeptide for delivery into one or more cells in vivo. The polypeptide can be any polypeptide. For example, the polypeptide can be a polypeptide that provides a therapeutic or prophylactic effect to an organism or that can be used to diagnose a disease or disorder in an organism. For example, for treatment of cancer, autoimmune disorders, parasitic, viral, bacterial, fungal or other infections, the polynucleotide(s) to be expressed may encode a polypeptide that functions as a ligand or receptor for cells of the immune system, or can function to stimulate or inhibit the immune system of an organism.

1. Pathogens

The antigen is typically obtained from, or raises a protective immune response to one or more pathogens, such as pathogens of the respiratory tract. In some embodiments, the antigen is a viral antigen. A viral antigen can be isolated from any virus. In an exemplary embodiment, the antigen is a natural viral capsid structure.

In some embodiments, the antigen is a bacterial antigen. Bacterial antigens can originate from any bacteria. In some embodiments the antigen is a parasite antigen. In some embodiments, the antigen is an allergen or environmental antigen. Exemplary allergens and environmental antigens, include but are not limited to, an antigen derived from naturally occurring allergens such as pollen allergens (tree-, herb, weed-, and grass pollen allergens), insect allergens (inhalant, saliva, and venom allergens), animal hair and dandruff allergens, and food allergens. In some embodiments, the antigen is a self-antigen such as in immune tolerance applications for auto-immune or related disorders such as Multiple Sclerosis. In some embodiments, the antigen is a tumor antigen. Exemplary tumor antigens include a tumor-associated or tumor-specific antigen. Preferred antigens include viral antigens obtained from respiratory pathogens, such as coronaviruses and influenza viruses.

In some embodiments, the antigen is an antigen obtained from, or which raises an immunological response to a mucosal pathogen, such as a virus, bacteria, nematode, protozoan, fungi or other pathogen that enters and/or infects the body through the mucosal surfaces.

a. Viral Antigens

In some embodiments, the antigen is a viral antigen isolated from a virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenza virus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens can be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

Exemplary viral antigens include E1A, E1B, E2, E3, E4, E5, L1, L2, L3, L4, L5 of Adenovirus; Pneumonoviridae (e.g., pneumovirus, human respiratory syncytial virus): Papovaviridae (polyomavirus and papillomavirus): E1, E2, E3, E4, E5a, E5b, E6, E7, E8, L1, L2; Human respiratory syncytial virus: human respiratory syncytial virus: G glycoprotein, RSV-viral proteins, e.g., RSV F glycoprotein; Dengue virus: core protein, matrix protein or other protein of Dengue virus; Measles: measles virus hemagglutinin; Herpesviridae (e.g., herpes simplex virus 1, herpes simplex virus 2, herpes simplex virus 5, and herpes simplex virus 6: herpes simplex virus type 2 glycoprotein gB, gB, gC, gD, and gE, HIV (GP-120, p17, GP-160, gag, pol, qp41, gp120, vif, tat, rev, nef, vpr, vpu, vpx antigens), ribonucleotide reductase, α-TIF, ICP4, ICP8, 1CP35, LAT-related proteins, gB, gC, gD, gE, gH, gI, gJ, and dD antigens; Lentivirus (e.g., human immunodeficiency virus 1 and human immunodeficiency virus 2): envelope glycoproteins of HIV I Picornaviridae (e.g., enterovirus, rhinovirus, hepatovirus (e.g., human hepatitis A virus); Cardiovirus; Apthovirus; Reoviridae (orthoreovirus, orbivirus, rotavirus, cypovirus, fijivirus, phytoreovirus, and oryzavirus), Retroviridae (mammalian type B retroviruses, mammalian type C retroviruses, avian type C retroviruses, type D retrovirus group, BLV-HTLV retroviruses); spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus); Poliovirus: I VP1; Hepatitis B virus: hepatitis B surface antigen hepatitis B virus core protein and/or hepatitis B virus surface antigen or a fragment or derivative thereof, hepatitis (Hep B Surface Antigen (gp27S, gp36S, gp42S, p22c, pol, x)). Additional viruses include Ebola, Marburg, Rabies, Hanta virus infection, West Nile virus, SARS-like Coronaviruses, Varicella-zoster virus, Epstein-Barr virus, Alpha viruse, St. Louis encephalitis, Adenovirdiae (mastadenovirus and aviadenovirus), Leviviridae (levivirus, enterobacteria phase MS2, allolevirus), Poxyiridae (e.g., chordopoxyirinae, parapoxvirus, avipoxvirus, capripoxvirus, leporipoxvirus, suipoxvirus, molluscipoxvirus, and entomopoxyirinae), Papovaviridae (polyomavirus and papillomavirus); Paramyxoviridae (paramyxovirus, parainfluenza virus 1), Mobillivirus (measles virus), Rubulavirus (mumps virus), metapneumovirus (e.g., avian pneumovirus and human metapneumovirus); Pseudorabies: pseudorabies virus g50 (gpD), pseudorabies virus II (gpB), pseudorabies virus gIII (gpC), pseudorabies virus glycoprotein H, pseudorabies virus glycoprotein E; transmissible gastroenteritis including transmissible gastroenteritis glycoprotein 195, transmissible gastroenteritis matrix protein;

Newcastle virus including Newcastle disease virus hemagglutinin-neuraminidase; infectious laryngotracheitis virus including viral antigens such as infectious laryngotracheitis virus glycoprotein G or glycoprotein 1; La Crosse virus including viral antigen such as a glycoprotein of La Crosse virus.

i. Coronavirus Antigens

In some embodiments, the antigen is derived from one or more coronaviruses. The coronaviruses (order Nidovirales, family Coronaviridae, and genus Coronavirus) are a diverse group of large, enveloped, positive-stranded RNA viruses that cause respiratory and enteric diseases in humans and other animals.

Coronaviruses typically have narrow host specificity and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group 1 (HCoV-229E) and group 2 (HCoV-OC43) and are historically responsible for ˜30% of mild upper respiratory tract illnesses.

At ˜30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini. The SARS-CoV rep gene, which includes approximately two-thirds of the genome, encodes two polyproteins (encoded by ORF1a and ORF1b) that undergo co-translational proteolytic processing. There are four open reading frames (ORFs) downstream of rep that are predicted to encode the structural proteins, S, E, M, and N, which are common to all known coronaviruses.

In some embodiments, the antigen is an antigen from a severe acute respiratory syndrome coronavirus-2 (SARS-CoV-2) betacoronavirus of the subgenus Sarbecovirus. SARS-CoV-2 viruses share approximately 79% genome sequence identity with the SARS-CoV virus identified in 2003. An exemplary nucleic acid sequence for the SARS-CoV-2 ORF1a/b gene is set forth in GenBank accession number MN908947.3. The genome organization of SARS-CoV-2 viruses is shared with other betacoronaviruses; six functional open reading frames (ORFs) are arranged in order from 5′ to 3′: replicase (ORF1a/ORF1b), spike (S), envelope (E), membrane (M) and nucleocapsid (N). In addition, seven putative ORFs encoding accessory proteins are interspersed between the structural genes.

In some preferred embodiments, the antigen includes one or more SARS-CoV-2 antigens from one or more of the genes encoding structural (S, E, M, N), or non-structural (NSPs) SARS-CoV-2 proteins. In some embodiments, the antigen includes one or more SARS-CoV-2 genes or gene expression products with selected epitopes in the SARS-CoV-2 genome that are conserved amongst multiple different coronaviruses. In further preferred embodiments, the antigen includes one or more structural (S, M, N) and non-structural (NSPs) SARS-CoV-2 proteins with selected epitopes in conserved regions of the SARS-CoV-2 genome, eliciting immune responses specific to the one or more SARS-CoV-2 antigens. In some embodiments the SARS-CoV-2 variant is selected from SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant).

ii. Influenza Virus Antigens

In some embodiments, the antigen is an influenza virus antigen. Influenza Virus antigens can be derived from a particular influenza clade or strain, or can be synthetic antigens, designed to correspond with highly conserved epitopes amongst multiple different influenza virus strains. There are four types of influenza viruses: A, B, C and D. Human influenza A and B viruses cause seasonal epidemics of disease. Influenza A viruses are the only influenza viruses known to cause flu pandemics, i.e., global epidemics of flu disease. Influenza type C infections generally cause mild illness and are not thought to cause human flu epidemics Influenza D viruses primarily affect cattle and are not known to infect or cause illness in people (see w.w.w.cdc.gov/flu/about/viruses/types.htm).

The influenza A virion is studded with glycoprotein spikes of hemagglutinin (HA) and neuraminidase (NA), in a ratio of approximately four to one, projecting from a host cell-derived lipid membrane. A smaller number of matrix (M2) ion channels traverse the lipid envelope, with an M2:HA ratio on the order of one M2 channel per 101-102 HA molecules. The envelope and its three integral membrane proteins HA, NA, and M2 overlay a matrix of M1 protein, which encloses the virion core. Internal to the M1 matrix are found the nuclear export protein (NEP; also called nonstructural protein 2, NS2) and the ribonucleoprotein (RNP) complex, which includes of the viral RNA segments coated with nucleoprotein (NP) and the heterotrimeric RNA-dependent RNA polymerase, composed of two “polymerase basic” and one “polymerase acidic” subunits (PB1, PB2, and PA). The organization of the influenza B virion is similar, with four envelope proteins: HA, NA, and, instead of M2, NB and BM2. Therefore, in some embodiments, the antigen is derived from one or more of the HA, NA, M2, NS2, NB, PB1, PB2, PA or NP genes of any influenza A or B virus. In particular embodiments, the antigen is derived from the NP gene of an influenza A or B virus (Bouvier and Palese P, Vaccine. 2008; 26 Suppl 4(Suppl 4):D49-D53. doi:10.1016/j.vaccine.2008.07.039).

The influenza A and B virus genomes each include eight negative-sense, single-stranded viral RNA (vRNA) segments, while influenza C virus has a seven-segment genome. The eight segments of influenza A and B viruses (and the seven segments of influenza C virus) are numbered in order of decreasing length. In influenza A and B viruses, segments 1, 3, 4, and 5 encode just one protein per segment: the PB2, PA, hemagglutinin (HA) and nucleoprotein (NP) proteins. All influenza viruses encode the polymerase subunit PB1 on segment 2; in some strains of influenza A virus, this segment also codes for the accessory protein PB1-F2, a small, 87-amino acid protein with pro-apoptotic activity, in a +1 alternate reading frame. No analogue to PB1-F2 has been identified in influenza B or C viruses. Conversely, segment 6 of the influenza A virus encodes only the NA protein, while that of influenza B virus encodes both the NA protein and, in a −1 alternate reading frame, the NB matrix protein, which is an integral membrane protein corresponding to the influenza A virus M2 protein. Segment 7 of both influenza A and B viruses code for the M1 matrix protein. In the influenza A genome, the M2 ion channel is also expressed from segment 7 by RNA splicing, while influenza B virus encodes its BM2 membrane protein in a +2 alternate reading frame. Finally, both influenza A and B viruses possess a single RNA segment, segment 8, from which they express the interferon-antagonist NS1 protein and, by mRNA splicing, the NEP/NS2, which is involved in viral RNP export from the host cell nucleus. Influenza A viruses are divided into subtypes based on hemagglutinin (H) and neuraminidase (N) proteins on the surface of the virus. There are 18 different hemagglutinin subtypes and 11 different neuraminidase subtypes (H1 through H18, and N1 through N11, respectively). Therefore, in some embodiments, the antigen is derived from the HA gene of an influenza virus influenza from any one or more of the H1 through H18 subtypes. In other embodiments, the antigen is derived from the NA gene of an influenza virus from any one or more of the N1 through N11 subtypes. While there are potentially 198 different influenza A subtype combinations, only 131 subtypes have been detected in nature. Current subtypes of influenza A viruses that routinely circulate in people include: A(H1N1) and A(H3N2). Therefore, in some embodiments, the antigen is derived from, or provides immunity to an A(H1N1) influenza virus, or an A(H3N2) influenza virus. In some embodiments, the antigen is conserved amongst, and/or provides immunity to all A(H1N1) influenza viruses. In some embodiments, the antigen is conserved amongst, and/or provides immunity to all A(H3N2) influenza viruses. In preferred embodiments, the antigen is conserved amongst, and/or provides immunity to both A(H1N1) influenza viruses and A(H3N2) influenza viruses.

Influenza A viruses are further classified into multiple subtypes (e.g., H1N1, or H3N2), while influenza B viruses are classified into one of two lineages: B/Yamagata and B/Victoria. Both influenza A and B viruses can be further classified into specific clades and sub-clades. Clades and sub-clades can be alternatively called “groups” and “sub-groups,” respectively. An influenza Glade or group is a further subdivision of influenza viruses (beyond subtypes or lineages) based on the similarity of their HA gene sequences. Clades and subclades are shown on phylogenetic trees as groups of viruses that usually have similar genetic changes (i.e., nucleotide or amino acid changes) and have a single common ancestor represented as a node in the tree. Clades and sub-clades that are genetically different from others are not necessarily antigenically different (i.e., viruses from a specific clade or sub-clade may not have changes that impact host immunity in comparison to other clades or sub-clades). In some embodiments, the antigen is conserved amongst, and/or provides immunity to two or more influenza viruses within the same subtype and/or sub-clade. In preferred embodiments, the antigen is conserved amongst, and/or provides immunity to two or more influenza viruses within different subtypes and/or sub-clades. In some embodiments, the antigen is conserved amongst, and/or provides immunity to all influenza viruses within the same subtype and/or sub-Glade. In preferred embodiments, the antigen is conserved amongst, and/or provides immunity to multiple influenza viruses within different subtypes and/or sub-clades.

Currently circulating influenza A(H1N1) viruses are related to the pandemic 2009 H1N1 virus that emerged in spring of 2009 and caused a flu pandemic (See w.w.w.cdc.gov/flu/about/viruses/types.htm). This virus is known as “A(H1N1)pdm09 virus,” or “2009 H1N1,” and continued to circulate seasonally from 2009 to 2021. These H1N1 viruses have undergone relatively small genetic changes and changes to their antigenic properties over time.

Of the influenza viruses that circulate and cause human disease, influenza A(H3N2) viruses tend to change more rapidly, both genetically and antigenically and have formed many separate, genetically different clades that continue to co-circulate. Therefore, in some embodiments, the antigen is derived from and/or provides immunity to all currently circulating H1N1 influenza viruses. In some embodiments, the antigen is derived from and/or provides immunity to all currently circulating H3N2 influenza viruses. In preferred embodiments, the antigen is derived from and/or provides immunity to all currently circulating H1N1 influenza viruses and H3N2 influenza viruses. In some embodiments, the antigen is derived from an Influenza A virus NP gene, or an Influenza A virus NP gene expression product.

Influenza B viruses are classified into two lineages: B/Yamagata and B/Victoria. Influenza B viruses are further classified into specific clades and sub-clades. Influenza B viruses change more slowly in terms of genetic and antigenic properties than influenza A viruses. Surveillance data from recent years shows co-circulation of influenza B viruses from both lineages in the United States and around the world with. Therefore, in some embodiments, the antigen is derived from and/or provides immunity to influenza B viruses. In some embodiments, the antigen is derived from and/or provides immunity to all currently circulating influenza B viruses. In some embodiments, the antigen is derived from an Influenza B virus NP gene, or an Influenza B virus NP gene expression product.

In some embodiments, the antigen is derived from and/or provides immunity to B/Yamagata and B/Victoria influenza viruses. In other embodiments, the antigen is derived from and/or provides immunity to one or more H1N1 influenza virus, and to one or more influenza B virus. In other embodiments, the antigen is derived from and/or provides immunity to one or more H3N2 influenza virus, and to one or more influenza B virus. In other embodiments, the antigen is derived from and/or provides mucosal immunity to one or more H1N1 influenza virus, to one or more H3N2 influenza virus, and to one or more influenza B virus.

Exemplary antigens include influenza virus hemagglutinin (Genbank accession No. J₀₂₁₃₂; Air, 1981, Proc. Natl. Acad. Sci. USA 78:7639-7643; Newton et al., 1983, Virology 128:495-501), influenza virus neuraminidase, PB1, PB2, PA, NP, M₁, M₂, NS₁, NS₂)) of Influenza virus; swine influenza including antigens such as swine flu hemagglutinin and swine flu neuraminidase. Exemplary equine viruses include equine influenza virus or equine herpesvirus: equine influenza virus type A/Alaska 91 neuraminidase, equine influenza virus type A/Miami 63 neuraminidase, equine influenza virus type A/Kentucky 81 neuraminidase. Exemplary cattle viruses include bovine parainfluenza virus type 3 fusion protein, and bovine parainfluenza virus type 3 hemagglutinin neuraminidase).

b. Bacterial Pathogens

In some embodiments, the antigen is a bacterial antigen. Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some embodiments, the antigenic or immunogenic protein fragment or epitope is derived from a pathogenic bacteria such as Anthrax; Chlamydia: Chlamydia protease-like activity factor (CPAF), major outer membrane protein (MOMP); Mycobacteria; Legioniella: Legionella peptidoglycan-associated lipoprotein (PAL), mip, flagella, OmpS, hsp60, major secretory protein (MSP); Diptheria: diptheria toxin; Streptococcus 24M epitope; Gonococcus: gonococcal pilin; Mycoplasm: mycoplasma hyopneumoniae; Mycobacterium tuberculosis: M. tuberculosis antigen 85A, 85B, MPT51, PPE44, mycobacterial 65-kDa heat shock protein (DNA-hsp65), 6-kDa early secretary antigenic target (ESAT-6); Salmonella typhi; Bacillus anthracis B. anthracis protective antigen (PA); Yersinia perstis: Y. pestis low calcium response protein V (LcrV), F1 and F1-V fusion protein; Francisella tularensis; Rickettsia typhi; Treponema pallidum; Salmonella: SpaO and H1a, outer membrane proteins (OMPs); and Pseudomonas: P. aeruginosa OMPs, PcrV, OprF, OprI, PilA and mutated ToxA.

c. Fungal Pathogens

In some embodiments, the antigenic or immunogenic protein fragment or epitope is derived from a pathogenic fungus, including, but not limited to, Coccidioides immitis: Coccidioides Ag2/Pra106, Prp2, phospholipase (P1b), alpha-mannosidase (Amn1), aspartyl protease, Gel1; Blastomyces dermatitidis: Blastomyces dermatitidis surface adhesin WI-1; Cryptococcus neoformans: Cryptococcus neoformans GXM and its Peptide mimotopes, and mannoproteins, Cryptosporidiums surface proteins gp15 and gp40, Cp23 antigen, p23; Candida spp. including C. albicans. C. glabrata. C. parapsilosis. C. dubliniensis. C. krusei. and others; Aspergillus species: Aspergillus Asp f 16, Asp f 2, Der p 1, and Fel d 1, rodlet A, PEP2, Aspergillus HSP90, 90-kDa catalase.

d. Protozoan or Parasitic Pathogens

In some embodiments, the antigenic or immunogenic protein fragment or epitope is derived from a pathogenic protozoan. Exemplary protozoa or protozoan antigens include: Plasmodium falciparum, Plasmodium vivax, Plasmodium ovale, Plasmodium malariae, Plasmodium apical membrane antigen 1 (AMA1), 25-kDa sexual-stage protein (Pfs25),-erythrocyte membrane protein 1 (PfEMP1) circumsporozoite protein (CSP), Merozoite Surface Protein-1 (MSP1); Leishmania species: Leishmania cysteine proteinase type III (CPC) Trypanosome species (African and American): T. pallidum outer membrane lipoproteins, Trypanosome beta-tubulin (STIB 806), microtubule-associate protein (MAP p15), cysteine proteases (CPs) Cryptosporidiums; isospora species; Naegleria fowleri; Acanthamoeba species; Balamuthia mandrillaris; Toxoplasma gondii, or Pneumocystis carinii: Pneumocystis carinii major surface glycoprotein (MSG), p55 antigen; Babesia Schistosomiasis: Schistosomiasis mansoni Sm14, 21.7 and SmFim antigen, Tegument Protein Sm29, 26 kDa GST, Schistosoma japonicum, SjCTPI, SjC23, Sj22.7, or SjGST-32 Toxoplasmosis: gondii surface antigen 1 (TgSAG1), protease inhibitor-1 (TgPI-1), surface-associated proteins MIC2, MIC3, ROP2, GRA1-GRA7.

2. Tumor Antigens

A tumor antigen may include a cell surface molecule. Tumor antigens of known structure and having a known or described function, include the following cell surface receptors: HER1 (GenBank Accession No. U48722), HER2 (Yoshino, et al., J. Immunol., 152:2393 (1994);); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. 573003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985)); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

3. Compositions for Transfection of Polynucleotides

It has been discovered that the gene delivery ability of polycationic polymers is due to multiple factors, including polymer molecular weight, hydrophobicity, and charge density. Many synthetic polycationic materials have been tested as vectors for non-viral gene delivery, but almost all are ineffective due to their low efficiency or high toxicity. Most polycationic vectors described previously exhibit high charge density, which has been considered a major requirement for effective DNA condensation. As a result, they are able to deliver genes with high efficiency in vitro but are limited for in vivo applications because of toxicity related to the excessive charge density.

High molecular weight polymers, particularly polymers, and methods of making them using enzyme-catalyzed copolymerization of a lactone with a dialkyl diester and an amino diol are disclosed. These poly(amine-co-ester) polymers have a low charge density. In addition, their hydrophobicity can be varied by selecting a lactone comonomer with specific ring size and by adjusting lactone content in the polymers. High molecular weight and increased hydrophobicity of the lactone-diester-amino diol polymers compensate for the low charge density to provide efficient gene delivery with minimal toxicity.

In preferred embodiments, the polymers exhibit efficient gene delivery with reduced toxicity. The polymers can be significantly more efficient the commercially available non-viral vectors. For examples, the polymers can be more than 100x more efficient than commercially available non-viral vectors such as PEI and LIPOFECTAMINE® 2000 based on luciferase expression assay while exhibiting minimal toxicity at doses of up to 0.5 mg/ml toxicity compared to these commercially available non-viral vectors. Preferably, the polymer is non-toxic at concentrations suitable for both in vitro and in vivo transfection of nucleic acids. For example, in some embodiments, the polymers cause less non-specific cell death compared to other approaches of cell transfection.

As described in more detail below, in some embodiments, the polymer is ω-pentadecalactone-diethyl sebacate-N-methyldiethanolamine polymer containing 20% PDL (also referred to as terpolymer III-20% PD).

4. Coating Agents for Particles

Efficiency of polynucleotide delivery using the polymers can be affected by the positive charges on the particle surface. For example, a zeta potential of the particle of +8.9 mV can attract and bind with negatively charged plasma proteins in the blood during circulation and lead to rapid clearance by the reticuloendothelial system (RES). Efficiency can also be affected by instability of the nanoparticles. For example, as discussed in the Examples below, particles incubated in NaAc buffer solution containing 10% serum nearly doubled in size within 15 minutes and increased by over 10-fold after 75 minutes. As a result of this increase in size, enlarged particles might be cleared from the circulation by uptake in the liver. Therefore, in some embodiments the particles are treated or coated to improve polynucleotide delivery efficiency. In some embodiments, the coating improves cell specific targeting of the particle, improves the stability (i.e., stabilizes the size of the particle in vivo), increases the half-life of the particle in vivo (i.e., in systemic circulation), or combinations thereof compared to a control. In some embodiments, the control is a particle without a coating.

An exemplary particle coating for targeting tumor cells is polyE-mRGD. As used herein, polyE-mRGD refers to a synthetic peptide containing three segments: a first segment including a polyglutamic acid (polyE) stretch, which is negatively charged at physiological pH and, therefore, capable of electrostatic binding to the positively charged surface of the particles; a second segment including a neutral polyglycine stretch, which serves as a neutral linker; and a third segment that includes a RGD sequence that binds the tumor endothelium through the interaction of RGD with αᵥβ₃ and αᵥβ₅.

As discussed in more detail below, the polyE-mRGD used in the Examples reversed the surface charge of 111-20% PDL/pLucDNA particle. When polyE-mRGD was added at 5:1 peptide/DNA weight ratio, the zeta potential of the particle changed from +8.9 mV to −5.8 mV. Peptide coated particles were stable upon incubation in NaAc buffer containing 10% serum and resistant to aggregation indicating that the modified particles can escape clearance by RES during circulation in vivo.

In one embodiment, polyE-mRGD includes the sequence EEEEEEEEEEEEEEEEGGGGGGRGDK (SEQ ID NO:1), or RGDKGGGGGG EEEEEEEEEEEEEEEE (SEQ ID NO:2), or a variant thereof with 85%, 90%, 95%, or more than 95% sequence identity to SEQ ID NO:1 or 2.

Another exemplary coating that can be used to prepare charge neutral, or negatively charged particles that maintain their size in vivo are described in Harris, et al., Biomaterials, 31:998-1006 (2010)), and can include the amino acid sequence GGGGGGEEEEEEEEEEEEEEEE (SEQ ID NO:3, poly-E), for non-specific systemic administration, or the amino acids sequence GdPdLGdVdRG-GGGGGG-EEEEEEEEEEEEEEEE-CONH2 (SEQ ID NO:4, poly-E-cat), which contains a polycationic sequence that increase targeting to the spleen, spine, sternum, and femur. In some embodiments, the polypeptide used in the coating is a variant SEQ ID NO:3 or 4, with 85%, 90%, 95%, or more than 95% sequence identity to SEQ ID NO:3 or 4

In vitro studies have indicated that adsorption of immunoglobulin G (IgG) and complement protein C3 to nanoparticles increases their uptake by Kupffer cells and incubation in serum increases hepatic uptake in vivo following liver perfusion (Nagayama, et al., Int. J. Pharm., 342:215-21 (2007)). Reports also indicate that galactose can be used to guide polymeric gene delivery particles to hepatocytes via the asialoglycoprotein receptor (Zhang, et al., J. Controlled Release, 102:749-63 (2005)).

5. Compositions for Altering Surface Charge

Polynucleotide delivery efficiency of the particles can be improved by coating the particles with an agent that is negatively charged at physiological pH. Preferably, the negatively charged agent is capable of electrostatic binding to the positively charged surface of the particles. The negatively charged agent can neutralize the charge of the particle, or reverse the charge of the particle. Therefore, in some embodiments, the negatively charged agent imparts a net negative charge to the particle.

In some embodiments, the negatively charged agent is a negatively charged polypeptide. For example, the polypeptide can include aspartic acids, glutamic acids, or a combination therefore, such that the overall charge of the polypeptide is a negative at neutral pH. In some embodiments, the polypeptide is a poly aspartic acid polypeptide including 3, 4, 5, 6, 7, 8, 9, 10, 11, 12, 13, 14, 15, 16, 17, 18, 19, 20, or more than 20 aspartic acid residues. In some embodiments, the polypeptide is a poly glutamic acid polypeptide including 3, 4, 5, 6, 7, 8, 9, 10, 11, 12, 13, 14, 15, 16, 17, 18, 19, 20, or more than 20 glutamic acid residues. Other negatively charged molecules include small molecules (i.e., MW less than 1500, 100, 750, or 500 Daltons) such as hyaluronic acid.

Increasing the negative charge on the surface of the particle can reduce or prevent the negative interactions described above, wherein more positively charged particles attract and bind negatively charged plasma proteins in the blood during circulation and lead to rapid clearance by the reticuloendothelial system (RES). In some embodiments, the zeta potential of the particles is from about −15 mV to about 10 mV, preferably from about −15 mV to about 8 mV, more preferably from about −10 mV to about 8 mV, more preferably from about −8 mV to about 8 mV. The zeta potential can be more negative or more positive than the ranges above provided the particles are stable (i.e., don't aggregate, etc.) and not readily cleared from the blood stream. The zeta potential can be manipulated by coating or functionalizing the particle surface with one or more moieties which varies the surface charge. Alternatively, the monomers themselves can be functionalized and/or additional monomers can be introduced into the polymer, which vary the surface charge.

6. Targeting Moieties

In some embodiments, the particles include a cell-type or cell-state specific targeting domain or targeting signal. Examples of moieties which may be linked or unlinked to the particles include, for example, targeting moieties which provide for the delivery of molecules to specific cells. The targeting signal or sequence can be specific for a host, tissue, organ, cell, organelle, non-nuclear organelle, or cellular compartment. For example, the compositions herein can be modified with galactosyl-terminating macromolecules to target the compositions to the liver or to liver cells. The modified compositions selectively enter hepatocytes after interaction of the carrier galactose residues with the asialoglycoprotein receptor present in large amounts and high affinity only on these cells. Moreover, the compositions here can be targeted to other specific intercellular regions, compartments, or cell types.

In one embodiment, the targeting signal binds to its ligand or receptor which is located on the surface of a target cell such as to bring the vector and cell membranes sufficiently close to each other to allow penetration of the vector into the cell. Additional embodiments are directed to specifically delivering polynucleotides to specific tissue or cell types, wherein the polynucleotides can encode a polypeptide or interfere with the expression of a different polynucleotide. The polynucleotides delivered to the cell can encode polypeptides that can enhance or contribute to the functioning of the cell.

The targeting moiety can be an antibody or antigen binding fragment thereof, an antibody domain, an antigen, a T-cell receptor, a cell surface receptor, a cell surface adhesion molecule, a major histocompatibility locus protein, a viral envelope protein and a peptide selected by phage display that binds specifically to a defined cell.

One skilled in the art will appreciate that the tropism of the particles described can be altered by merely changing the targeting signal. It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used as targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest.

7. Size of Particles

Resistance to aggregation can be important because maintaining a small particle size limits clearance by the liver and maintains transfection ability of particles into target cells. Therefore, in preferred embodiments, the particles are resistant to aggregation. Preferably, particles with or without coating are between about 1 nm and 1000 nm in radius, more preferably between about 1 nm and about 500 nm in radius, most preferably between about 15 nm and about 250 nm in radius. For example, in some embodiments, coated particles loaded with polynucleotide are between about 150 nm and 275 nm in radius.

The ratio of polynucleotide weight to polymer weight (polynucletide:polymer), the content and quantity of particle coating, or a combination thereof can be used to adjust the size of the particles.

For example, the Examples below show that in some embodiments, transfection efficiency of particles with 25:1 polymer to DNA ratio is lower than the transfection efficiency of particles with 50:1, 100:1, 150:1, and 200:1 polymer:DNA ratios. The most preferred polymer:polynucleotide ratio for a particular formulation can be determined empirically using the methods that are known in the art, such as those described in the Examples below. Generally, the weight:weight ratio of polymer:polynucleotide is preferably greater than about 10:1, more preferably greater than about 50:1, most preferably greater than about 100:1. The weight:weight ratio of polymer:polynucleotide is preferably between about 10:1 and 500:1, more preferably between about 25:1 and 250:1, most preferably between about 50:1 and 150:1. In some embodiments, the weight ratio of polymer:polynucleotide is about 100:1. Preferably, the particles have are spherical in shape.

The examples also show that in some embodiments, transfection efficiency of particles by the ratio of coating agent molecules to polynucleotide molecules (coating agent:polynucleotide). The ratio is expressed by weight. The most preferred coating agent:polynucleotide ratio for a particular formulation can be determined empirically using the methods that are known in the art, such as those described in the Examples below. Generally, the ratio of coating agent:polynucleotide is greater than 0, and preferably lower than about 50:1, more preferably lower than about 25:1, most preferably lower than about 10:1. The ratio coating agent:polynucleotide is preferably between about 1:1 and 10:1, more preferably between about 2.5:1 and 7.5:1. In some embodiments, the ratio of coating agent:polynucleotide is about 5:1. Ratios of coating agent:polynucleotide of 10:1, 5:1, and 2.5:1 are also referred to herein as 10×, 5×, and 2.5× respectively. Preferably, the particles are spherical in shape.

8. PEG-Blocking Containing Polymers

The polymers can be used for drug delivery, for example, in the formation of particles, such as microparticles or nanoparticles, or micelles which can release one or more therapeutic, prophylactic, and/or diagnostic agents in a controlled release manner over a desirable period of time.

pH-responsive micelle nanocarriers are often formed via self-assembly of amphiphilic block copolymers and include a hydrophilic (e.g., PEG) outer shell and a hydrophobic inner core capable of response to medium pH. Typically, upon changing the medium pH from neutral or slightly basic to mildly acidic, the micelle cores undergo accelerated degradation, become completely soluble in water, or swell substantially in aqueous medium. As the result, the drug-encapsulated micelles with a slow drug-release rate at the physiological pH can be triggered by an acidic pH to rapidly unload the drug molecules. The polymer segments constituting the micelle cores in previous reports include poly(ortho esters), poly(β-amino esters), poly(L-histidine), and others. The major disadvantages with most of the previous micelle systems are the multiple steps required for preparing the copolymers and the difficulty of controlling the polymer molecular weight and adjusting the polymer composition during the copolymer synthesis.

The copolymers exhibited variation in the rate of release as a function of pH. In vitro drug release behaviors of the DTX-encapsulated micelles of PEG2K-PPMS copolymer samples (PEG2K-PPMS-11% PDL, PEG2K-PPMS-30% PDL, and PEG2K-PPMS-51% PDL) were studied in PBS solution at both physiological pH of 7.4 and acidic pH of 5.0. In general, the DTX release from all micelle samples followed biphasic release kinetics and exhibited remarkable pH-dependence. The DTX-loaded PEG2K-PPMS copolymer micelles release 25-45% drug rapidly during the initial 12 h, followed by a more gradual release of additional 25-40% drug for the subsequent 132 h. The influence of the medium pH on the drug release rate is substantial. For example, at the end of the incubation period (144 h), the values of accumulated DTX released from the micelles of PEG2K-PPMS-11% PDL, PEG2K-PPMS-30% PDL, and PEG2K-PPMS-51% PDL copolymers are respectively 66%, 60%, and 55% at physiological pH of 7.4, which increase correspondingly to 85%, 81%, and 75% at acidic pH of 5.0. The observed pH-triggered acceleration of DTX release from the PEG2K-PPMS copolymer micelles is consistent with the earlier observation that changing of the medium pH from 7.4 to 5.0 causes significant swelling of the micelles due to the protonation and size increase of the micelle PPMS cores. This pH-triggered micelle size expansion would certainly facilitate the diffusion and release of entrapped DTX from the micelle cores to the aqueous medium. At a given pH, the DTX release rate is presumably controlled by the interactions between the drug and the PPMS matrix in the micelle cores. Since PDL-rich PEG2K-PPMS copolymers are expected to form strong hydrophobic domains in their micelle inner cores to better trap and retain hydrophobic DTX molecules, the drug release from such copolymer micelles should be more gradual and sustained. This hypothesis is supported by the experimental result showing that at both pH of 7.4 and 5.0, the DTX release rate from PEG2K-PPMS copolymer micelles decreases with increasing PDL content in the PPMS chain segments of the copolymer.

It is known that upon uptake of micelles by tumor cells, the micelle particles are subjected to entrapment in endosomes with pH ranging from 5.5 to 6.0 and in lysosomes with pH ranging from 4.5 to 5.0. As the above results clearly show, these acidic environments would inevitably trigger fast DTX release from PEG2K-PPMS copolymer micelles, thus enhancing the cytotoxicity of the drug-loaded micelles. The amino groups in the copolymers would act as proton sponges to facilitate endosomal escape. Therefore, the pH-responsive properties exhibited by the PEG2K-PPMS copolymer micelles are highly desirable, which render them to be superior carriers for delivery of anticancer drugs.

### III. Formulations

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients.

There are two general classes of formulations that can be used for administration of antigen, which is preferably in the form of a nucleic acid that encodes the antigen. In the more preferred embodiment, the nucleic acid is an mRNA. The two general classes of formulations are (1) polymeric nanoparticles formed of PACE and (2) lipid based particles, lipid emulsions, and liposomes.

A variety of materials have been developed for mRNA delivery, including lipids, lipid-like materials, polymers and protein derivatives. Preferred lipids include Distearoylphosphatidylcholine (DSPC), dipalmitoyl phosphatidylcholine, phosphatidylserine, cholesterol and polyethylene glycol (PEG)-lipid. Other materials include squalene, Span 85, DOTAP, polysorbate 80, trioleate and other surfactants.

These include liposomes, liposome-like nanoparticles, solid lipid nanoparticles, nanostructured lipid carriers, absorbed lipid-polymer hybrid nanoparticles, encapsulated lipid-polymer hybrid nanoparticles, cation nanoemulsions, exosomes, native lipoprotein, and synthetic lipoprotein, Different lipids have been commonly used to fabricate various lipid-based formulations for the delivery of nucleic acids. Traditional liposomes, lipoplexes, cationic nanoemulsions (CNEs), and nanostructured lipid carriers (NLCs) were developed as delivery systems for nucleic acids.

Liposomes are spherical vesicles comprising unilamellar or multilamellar phospholipid bilayers enclosing an aqueous core in which the drug of choice can be encapsulated. They are prepared from materials possessing polar head (hydrophilic) groups and nonpolar tail (hydrophobic) groups. The interaction between these groups induces the formation of vesicles. Pharmaceutics 13, 206 (2021 https://doi.org/10.3390/pharmaceutics13020206.

In particular, lipid nanoparticles have been thoroughly investigated and successfully entered into the clinic for the delivery of small molecules, siRNA drugs and mRNA. Notably, two authorized coronavirus disease 2019 (COVID-19) vaccines, mRNA-1273 and BNT162b, use lipid nanoparticles to deliver antigen mRNA. Many other lipid nanoparticle-mRNA formulations have been developed and are under clinical evaluation for the prevention and treatment of virus infections, cancer and genetic diseases. Suitable lipid nanoparticles for mRNA delivery are known. See, for example, Hou, et al. Nature Reviews Materials 6, 1078-1094 (2021), A review of lipid carriers for mRNA can also be found at Zhang, et al. Acta Pharmaceutica Sinica B doi.org/10.1016/j.apsb.2022.11.026, See also Bolhassani, A. Lipid-Based Delivery Systems in Development of Genetic and Subunit Vaccines. Mol Biotechnol (2022). https://doi.org/10.1007/s12033-022-00624-8.

More advanced delivery systems of LNPs are more effective for delivering nucleic acids compared to the classical lipid-based formulations. These advanced LNPs may not show a lipid bilayer enclosing an aqueous core. Instead, they may present a micelle-like structure that encapsulates drug molecules inside a non-aqueous core. In addition, LNPs do not exhibit electrostatic complexation with their nucleic acid contents.

Gerhardt, et al. Mol. Ther. Method Clin. Dev. 25, 205 (2022) describes a. thermostable nanostructured lipid carrier for delivery of mRNA vaccines. These have diameters of less than 150 nm.

Solid lipid nanoparticles (SLN) are a particulate carrier system composed of a solid lipid core and a cationic lipid surface suitable for binding negatively, charged DNA. SLN delivery systems can be used to bind DNA resulting in an SLN/DNA complex (termed. “lipoplex”) which can be used as a potential vaccine. Francis, et al. (2022). Design and Preparation of Solid Lipid Nanoparticle (SLN)-Mediated DNA Vaccines. In: Thomas, S. (eds) Vaccine Design. Methods in Molecular Biology, vol 2412. Humana, New York, N.Y. doi.org/10.1007/978-1-0716-1892-9_18, See also Aldosari, et al. Lipid Nanoparticles as Delivery Systems for RNA-Based Vaccines. Pharmaceutics 2021, 13, 206.https://doi.org/10.3390/pharmaceutics1302020.

### IV. Methods of Preparing Polymeric Nanoparticles

A. Methods for Making Particles

Particles can be prepared using a variety of techniques known in the art. The technique to be used can depend on a variety of factors including the polymer used to form the nanoparticles, the desired size range of the resulting particles, and suitability for the material to be encapsulated.

Methods known in the art that can be used to prepare nanoparticles include, but are not limited to, polyelectrolyte condensation (see Suk et al., Biomaterials, 27, 5143-5150 (2006)); single and double emulsion; nanoparticle molding, and electrostatic self-assembly (e.g., polyethylene imine-DNA or liposomes). Preferred methods also include microfluidic methods (see, for example, Liu, et al., Nano Micro Small Review (2019); Anal. Chem. 2018, 90, 3, 1434-1443 (2017); Lim, et al., “Ultra-High Throughput Synthesis of Nanoparticles with Homogeneous Size Distribution Using a Coaxial Turbulent Jet Mixer” ACS Nano 2014, 8, 6, 6056-6065 (2014)). These also work for PACE nanoparticles.

In one embodiment, the loaded particles are prepared by combining a solution of the polymer, typically in an organic solvent, with the polynucleotide of interest. The polymer solution is prepared by dissolving or suspending the polymer in a solvent. The solvent should be selected so that it does not adversely affect (e.g., destabilize or degrade) the nucleic acid to be encapsulated. Suitable solvents include, but are not limited to, DMSO and methylene chloride. The concentration of the polymer in the solvent can be varied as needed. In some embodiments, the concentration is for example 25 mg/ml. The polymer solution can also be diluted in a buffer, for example, sodium acetate buffer.

Next, the polymer solution is mixed with the agent to be encapsulated, such as a polynucleotide. The agent can be dissolved in a solvent to form a solution before combining it with the polymer solution. In some embodiments, the agent is dissolved in a physiological buffer before combining it with the polymer solution. The ratio of polymer solution volume to agent solution volume can be 1:1. The combination of polymer and agent are typically incubated for a few minutes to form particles before using the solution for its desired purpose, such as transfection. For example, a polymer/polynucleotide solution can be incubated for 2, 5, 10, or more than 10 minutes before using the solution for transfection. The incubation can be at room temperature.

In some embodiments, the particles are also incubated with a solution containing a coating agent prior to use. The particle solution can be incubated with the coating agent for 2, 5, 10, or more than 10 minutes before using the particles for transfection. The incubation can be at room temperature.

In some embodiments, if the agent is a polynucleotide, the polynucleotide is first complexed to a polycation before mixing with polymer. Complexation can be achieved by mixing the polynucleotides and polycations at an appropriate molar ratio. When a polyamine is used as the polycation species, it is useful to determine the molar ratio of the polyamine nitrogen to the polynucleotide phosphate (N/P ratio). In a preferred embodiment, inhibitory RNAs and polyamines are mixed together to form a complex at an N/P ratio of between approximately 1:1 to 1:25, preferably between about 8:1 to 15:1. The volume of polyamine solution required to achieve particular molar ratios can be determined according to the following formula:

$${V}_{NH 2} = \frac{{C}_{inhRNA , final} \times {M}_{w , inhRNA} / {C}_{inhRNA , final} \times {M}_{w , P} \times {Φ}_{N : P} \times Φ {V}_{final}}{{C}_{NH 2} / {M}_{w , NH 2}}$$

where M_{w,inhRNA}=molecular weight of inhibitory RNA, M_{w,P}=molecular weight of phosphate groups of inhibitory RNA, Φ_{N:P}=N:P ratio (molar ratio of nitrogens from polyamine to the ratio of phosphates from the inhibitory RNA), C_{NH2}, stock=concentration of polyamine stock solution, and M_{w,NH2}=molecular weight per nitrogen of polyamine. Methods of mixing polynucleotides with polycations to condense the polynucleotide are known in the art. See for example U.S. Published Application No. 2011/0008451.

The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values. Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In some embodiments, the polycation is a polyamine. Polyamines are compounds having two or more primary amine groups. Suitable naturally occurring polyamines include, but are not limited to, spermine, spermidine, cadaverine and putrescine. In a preferred embodiment, the polyamine is spermidine.

In another embodiment, the polycation is a cyclic polyamine. Cyclic polyamines are known in the art and are described, for example, in U.S. Pat. No. 5,698,546, WO 1993/012096 and WO 2002/010142. Exemplary cyclic polyamines include, but are not limited to, cyclen.

Spermine and spermidine are derivatives of putrescine (1,4-diaminobutane) which is produced from L-ornithine by action of ODC (ornithine decarboxylase). L-ornithine is the product of L-arginine degradation by arginase. Spermidine is a triamine structure that is produced by spermidine synthase (SpdS) which catalyzes monoalkylation of putrescine (1,4-diaminobutane) with decarboxylated S-adenosylmethionine (dcAdoMet) 3-aminopropyl donor. The formal alkylation of both amino groups of putrescine with the 3-aminopropyl donor yields the symmetrical tetraamine spermine. The biosynthesis of spermine proceeds to spermidine by the effect of spermine synthase (SpmS) in the presence of dcAdoMet. The 3-aminopropyl donor (dcAdoMet) is derived from S-adenosylmethionine by sequential transformation of L-methionine by methionine adenosyltransferase followed by decarboxylation by AdoMetDC (S-adenosylmethionine decarboxylase). Hence, putrescine, spermidine and spermine are metabolites derived from the amino acids L-arginine (L-ornithine, putrescine) and L-methionine (dcAdoMet, aminopropyl donor).

### V. Methods of Use as to Elicit an Immune Response/as Vaccines

It has been established that PACE particles including antigens, or nucleic acids encoding antigens can be used to provide enhanced mucosal immunity against pathogens. The antigen-containing PACE particles can be used in a two-step vaccination strategy against infection by a virus, termed “prime and PACE-spike”.

Natural infection induces the development of tissue-resident mucosal adaptive immunity that act as sentinels at the site of pathogen encounter, including 1) CD8 T cells that can quickly kill infected cells preventing amplification and spread of a pathogen within an infected person; 2) B cells that produce specific mucosal antibodies called secretory IgA that resides at the mucosa to prevent pathogen entry and infection; and 3) CD4 T cells that can help CD8 T cells and B cells to function optimally. Parenteral vaccination given as intramuscular injection (Shots) provides excellent systemic immune protection but does not induce robust mucosal immunity. Mucosal exposure to cognate antigens and/or inflammation is key to the development of tissue-resident memory T and B cell responses.

By “mucosal immune response” or “mucosal immunity” as the terms are interchangeably used, is meant the induction of a humoral (i.e., B cell and antibodies) and/or cellular (i.e., T cell) response. Most preferably, this immune response is specific for the antigen with which the mammalian host was immunized. Suitably, a humoral mucosal immune response may be assessed by measuring the antigen-specific antibodies present in the mucosal lavage in response to introduction of the desired antigen into the host. In one exemplary embodiment, the mucosal immune response is assessed by measuring anti-antigen antibody titers and isotype profiles in Bronchoalveolar Lavage (B AL) fluid of immunized subjects. Most preferably, the antibody response is composed primarily of IgA antibodies. A cellular mucosal immune response may be assessed by measuring the T cell response within tissues isolated from the mucosal area (e.g., respiratory, or gastrointestinal tract) or from lymph nodes that drain from the mucosal area (for example respiratory area or gastrointestinal area). The method is not limited to respiratory mucosal immune response. In some embodiments, the immune response is one at various mucosa of mammals, preferably humans.

Methods for inducing or enhancing a robust mucosal immunity to an exogenous antigen in mucosal and epithelial tissues of a subject are provided. The methods effectively generate one or more populations of tissue resident T cells and B cells in the recipient. In preferred embodiments, the methods provide a population of CD8⁺ tissue-resident memory (T_{RM}) cells, CD4⁺ tissue-resident memory (T_{RM}) cells, and/or memory B cells at the mucosal tissue, protective against the vaccinating antigen(s) and/or the original host where the vaccinating antigen is sourced. In one embodiment, methods induce a population of CD69⁺CD8⁺ tissue-resident memory (T_{RM}) cells against the vaccinating antigen(s) in the lung and/or mediastinal lymph node. In one embodiment, methods induce a population of CD69⁺CD103⁺CD8⁺ tissue-resident memory (T_{RM}) cells against the vaccinating antigen(s) in the lung and/or mediastinal lymph node. In a further embodiment, methods induce one or more populations of CXCR5⁺PD1⁺ follicular helper CD4⁺ T cells (T_{fh} cells), GL7⁺ germinal center B cells, CD138+ antibody-secreting cells (ASC), and antigen-specific B cells in the draining lymph node. In some embodiments, the method provides an effective increase in IgA and/or IgG antibodies against the vaccinating antigen(s) in BALF and/or in the serum.

A. Methods of Priming and Boosting

In some aspects, the present invention is directed to a method of treating, ameliorating, and/or preventing an infection by a virus in a subject in need thereof. In some aspects, the present invention is directed to a method of immunizing a mammal against an infection by a virus. In some embodiments, the virus is one that causes a respiratory infection. In some embodiments, the virus is a coronavirus, an influenza virus, a herpes simplex virus, or any combinations thereof.

In some embodiments, the existing immunity is obtained by a “priming” vaccination, such as those described elsewhere herein. In some embodiments, the existing immunity is obtained via a prior infection by a coronavirus.

In some embodiments, the method includes administering intranasally to the subject a composition including a coronavirus protein. In some embodiments, the composition is the same as or similar to those used in the “spiking” step as described elsewhere herein.

The examples described herein (“the present study”) demonstrated the following two-step vaccination strategies against infection by a virus (using SARS-CoV-2 as a non-limiting example: (1) IM prime and IN boosting with antigen (herein, “prime and spike”); (2) IM prime and IN boosting with antigen-containing PACE/antigen encapsulating PACE (herein, prime and PACE-spike); (3) IM prime and IM boosting with antigen-containing PACE/antigen encapsulating PACE (herein, prime and IM PACE-spike (4) IN prime and IN boosting both with antigen-containing PACE/antigen encapsulating PACE (herein “IN PACE-prime and IN PACE-spike”). IM prime as used herein refers to a conventional parenterally administered vaccine (e.g., intramuscular administration of an mRNA-lipid nanoparticle (LNP)-based SARS-CoV-2 vaccine).

In some embodiments, the method includes the steps of i) administering via a systemic or mucosal route of administration an effective amount of a priming immunogenic composition and subsequently ii) administering via a mucosal route to the subject an effective amount of a boosting immunogenic composition. This method is based on the discovery that systemic or mucosal priming of a subject using a vaccine expressing an antigen selectively augments or enhances the induction of antigen-specific mucosal immunity upon subsequent immunization or boosting, via a mucosal route, with a composition containing the same antigen or a fragment thereof. The present study demonstrated that “priming” the subject (e.g., a mouse) with a conventional parenterally administered vaccine (e.g., intramuscular administration of an mRNA-lipid nanoparticle (LNP)-based SARS-CoV-2 vaccine), followed by “spiking” the subject with (i) an intranasally administered viral protein (e.g., intranasal administration of SARS-CoV-2 spike protein) or (ii) intranasally administered antigen-containing PACE particles, resulted in robust immune response. The immune responses elicited by the “prime and spike” or ‘prime and PACE-spike” strategy are more robust than those elicited by the conventional “prime and boost” vaccination strategy (two or more parenteral administration of vaccines). The present study also demonstrated that “priming” and spiking the subject (e.g., a mouse) with antigen-containing PACE intranasally administered antigen-containing PACE particles, resulted in robust immune response. Similarly, priming the subject and “spiking” the subject with IN antigen or antigen-containing PACE particles or IM antigen-containing PACE particles within the context of waning immunity protects against a lethal SARS-CoV-2 Challenge.

The method of the invention can be readily adapted to induce or enhance mucosal immunity in a host to any antigen and preferably, confers protective immunity.

i. Prime and Spike

In some embodiments, the method includes: administering parenterally to the subject a vaccination against the virus; and administering intranasally a viral protein to the subject. In some embodiments, the viral protein comprises an isolated viral protein. In some embodiments, the viral protein comprises an isolated spike protein of a coronavirus.

In some embodiments, administering intranasally the viral protein to the subject includes administering a spike protein of a coronavirus. In some embodiments, the spike protein is from the same type of coronavirus being treated, prevented and/or ameliorated, and/or being vaccinated against. In some embodiments, the spike protein is from a different type of coronavirus because, due to the similarities in amino acid sequences, protein structures, and/or glycosylation patterns of spike proteins among the coronavirus family, immunities elicited by a spike protein from a first coronavirus is often effective against a second coronavirus. Indeed, the present study has demonstrated that “spiking” a subject with either SARS-CoV spike protein or SARS-CoV-2 spike protein is able to generate pan-sarbecovirus immunity in the subject. Specifically, the present study demonstrates that “spiking” an already “primed” subject with a recombinant SARS-CoV spike protein is able to elicit robust production of anti-SARS-CoV-2 IgA and IgG in the subject. Similarly, “spiking” an already “primed” subject with a recombinant SARS-CoV-2 spike protein is able to elicit robust production of anti-SARS-CoV IgA and IgG in the subject, as well. In some embodiments, the spike protein is a recombinant SARS-CoV-2 spike protein (either wild-type or genetically engineered, such as genetically engineered to improve the stability or immunogenicity to the spike of the same or different coronaviruses), or a fragment thereof. In some embodiments, the spike protein is a recombinant SARS-CoV spike protein (either wild-type or genetically engineered, such as genetically engineered to improve the stability or immunogenicity to the spike of the same or different coronaviruses), or a fragment thereof. In some embodiments, the SARS-CoV spike proteins, the SARS-CoV-2 spike proteins, or the fragments thereof are glycosylated. In some embodiments, the SARS-CoV spike proteins, the SARS-CoV-2 spike proteins, or the fragments thereof are used to “spike” a subject to boost an immunity against a SARS-CoV infection, a SARS-CoV-2 infection, both, or other types of coronavirus infections.

In some embodiments, the antigenic viral protein such as spike protein is administered intranasally without an adjuvant. In some embodiments, the antigenic viral protein such as spike protein is administered intranasally with one or more adjuvants. In some embodiments, the antigenic viral protein such as spike protein is administered with one or more pharmaceutically acceptable carrier suitable for intranasal administration.

ii. Antigen-Containing Prime and PACE-Spike

In one embodiment, the method includes the steps of i) administering via an intramuscular route of administration an effective amount of a priming immunogenic composition including mRNA encoding one or more antigens and subsequently ii) administering via a mucosal route to the subject an effective amount of a boosting immunogenic composition including mRNA encoding the same antigens to establish tissue-resident immunity. In one embodiment, the methods include steps of: (i) administering via intramuscular (IM) injection an effective amount of a priming antigen containing lipid nanoparticles (LNP) and a polynucleotide encoding the antigen; and (ii) subsequently administering intranasally (IN) or IM to the subject, an effective amount of a boosting composition containing poly(amine-co-ester) or poly(amine-co-amide), (PACE), PACE-PEG block copolymer and/or PACE polymer blend which may include additional monomers to make the nanoparticles more acid sensitive, and a polynucleotide encoding the same antigen. In another embodiment, the method includes the steps of i) administering via a mucosal route of administration an effective amount of a priming immunogenic composition including mRNA encoding one or more antigens and subsequently ii) administering via a mucosal route to the subject an effective amount of a boosting immunogenic composition including mRNA encoding the same antigens to establish tissue-resident immunity. In one embodiment, the methods include the step of administering intranasally (IN) an effective amount of a priming antigen composition; and (ii) subsequently administering intranasally (IN) to the subject an effective amount of a boosting antigen composition. In preferred embodiments, the priming immunogenic composition and/or the boosting immunogenic composition contain poly(amine-co-ester) or poly(amine-co-amide), (PACE), PACE-PEG block copolymer and/or PACE polymer blend which may include additional monomers to make the nanoparticles more acid sensitive, and a polynucleotide encoding the same antigen, preferably, encapsulated in the nanoparticles.

In some embodiments, the method for inducing a protective immunity includes the steps of i) administering via a systemic route of administration an effective amount of a priming immunogenic composition and subsequently ii) administering via a systemic or mucosal route to the subject an effective amount of a boosting immunogenic composition. In one embodiment, the methods include steps of: (i) administering via intramuscular (IM) injection an effective amount of a priming antigen containing lipid nanoparticles (LNP) and a polynucleotide encoding the antigen; and (ii) subsequently via intramuscular (IM) injection to the subject an effective amount of a boosting composition containing poly(amine-co-ester) or poly(amine-co-amide), (PACE), PACE-PEG block copolymer and/or PACE polymer blend which may include additional monomers to make the nanoparticles more acid sensitive, and a polynucleotide encoding the same antigen. The methods are effective in providing protective immunity again one or more respiratory pathogens.

In further embodiments, the method for inducing a protective immunity includes the steps of i) administering via a mucosal route of administration an effective amount of a priming immunogenic composition and subsequently ii) administering via a systemic route to the subject an effective amount of a boosting immunogenic composition. In preferred embodiments, the priming immunogenic composition and/or the boosting immunogenic composition contain poly(amine-co-ester) or poly(amine-co-amide), (PACE), PACE-PEG block copolymer and/or PACE polymer blend which may include additional monomers to make the nanoparticles more acid sensitive, and a polynucleotide encoding the same antigen.

In preferred embodiments, the immunogenic composition for administration to the mucosal surface includes poly(amine-co-ester) particles for effective delivery of mRNA. The mucosal immunity is enhanced compared that induced by without poly(amine-co-ester) particles, for example, in terms of the number of tissue resident memory T cells, tissue resident memory B cells, and mucosal IgA against one or more vaccinating antigens at a mucosal site, particularly in the respiratory tract.

The priming or boosting composition when administered via a mucosal route is administered via a selected mucosa. The preferred route of mucosal administration is the intranasal route. Delivery can be accomplished by aerosol, nebulizer, or by depositing a liquid in the nasal cavity.

In some embodiments, the priming includes previous exposure to the antigen via a mucosal route. For example, in some embodiments, the subject is a subject who has previously been exposed to the antigen via administration of an effective amount of a immunogenic composition comprising the antigen to mucosal tissue to the subject. Exemplary mucosal tissues include pulmonary, nasal, oral, gastrointestinal, vaginal, and rectal mucosa.

In some embodiments, administering parenterally to the subject the vaccination against the coronavirus elicits systemic T cell, B cell, and antibody responses in the subject.

In some embodiments, administering intranasally the viral protein to the subject establishes lung-resident T and B cells. In some embodiments, the vaccination administering parenterally to the subject includes any vaccines against any coronaviruses that are suitable for parenteral administration. In some embodiments, the vaccination administering parenterally to the subject includes an mRNA-lipid nanoparticle (LNP)-based vaccine, a viral vector vaccine (such as a non-replicating or a replicating viral vector vaccine), an inactivated virus vaccine, a viral subunit/peptide vaccine, a DNA vaccine, a virus-like particle (VLP) vaccine, or the like.

Examples of mRNA-LNP based vaccines against SARS-CoV-2 virus include BNT162b1 and BNT162b2 (COMIRNATY/Tozinameran) and BNT162b2s01 by Pfizer and BioNTech, mRNA-1273 (SPIKEVAX/elasomeran) and mRNA-1273.211 and mRNA-1273.617.2 by Moderna, CVnCoV and CV2CoV by CureVac, ARCT-154 by Arcturus Therapeutics Inc, ARCoV by Abogen, TAK-919 (Moderna formulation) by Takeda, mRNA by Walvax, and the like.

Examples of non-replicating viral vector vaccines against SARS-CoV-2 virus include Ad26.COV2.S by Janssen (Johnson & Johnson), AZD1222 and AZD2816 developed by Oxford University and AstraZeneca, Gam-COVID-Vac (Sputnik V) and Sputnik Light by Gamaleya Research Institute of Epidemiology and Microbiology in Russia, AD5-nCOV and AD5-nCOV-IH developed by CanSino Biologics, Covishield (Oxford/AstraZeneca formulation) by Serum Institute of India, GRAd-COV2 by ReiThera, and the like.

Examples of replicating viral vector vaccines against SARS-CoV-2 virus include DelNS1-2019-nCoV-RBD-OPT by Wantai, IIBR-100 by Israel Institute for Biological Research (IIBR), and the like.

Examples of inactivated virus vaccines against SARS-CoV-2 virus include CoronaVac by Sinovac Biotech, BBIBP-CorV and WIBP-CorV by Sinopharm, Covaxin by Bharat Biotech, KoviVac by Chumakov Centre, COVIran Barekat by Shifa Pharmed Industrial Group, VLA2001 and VLA2101 by Valneva SE and Dynavax Technologies, ERUCOV-VAC by Health Institutes of Turkey, QazVac by Kazakhstan RIBSP, SARS-CoV-2 Vaccine (Vero Cells) by Minhai Biotechnology Co, FAKHRAVAC (MIVAC) by Organization of Defensive Innovation and Research, KD-414 by KM Biologics Co Ltd, Inactivated (Vero Cells) by Chinese Academy of Medical Sciences, and the like.

Examples of viral subunit/peptide vaccines against SARS-CoV-2 virus include EpiVacCorona by the VECTOR center of Virology, ZF2001 by Anhui Zhifei Longcom and the Institute of Microbiology at the Chinese Academy of Sciences, MVC COVID-19 vaccine by Medigen Vaccine Biologics Corporation, Dynavax Technologies and the U.S. National Institutes of Health, Novavax COVID-19 vaccine (NVX-CoV2373) by Novavax and the Coalition for Epidemic Preparedness Innovations (CEPI), FINLAY-FR-2 by Finlay Institute, VAT00002 and VAT00008 by Sanofi Pasteur and GlaxoSmithKline, IVX-411 and IVX-421 by Icosavax, SPFN_1B-06-PL by Walter Reed Army Institute of Research (WRAIR), SCB-2019 (CpG 1018/Alum) by Clover Biopharmaceuticals, BECOV2A/Corbevax by Biological E Limited, CIGB-66 by Center for Genetic Engineering and Biotechnology (CIGB), EpiVacCorona and EpiVacCorona-N by FBRI, Soberana 02 and Soberana Plus by Instituto Finlay de Vacunas Cuba, Razi Coy Pars by Razi Vaccine and Serum Research Institute, COVOVAX (Novavax formulation) by Serum Institute of India, COVAX-19 by Vaxine/CinnaGen Co., Nanocovax by Nanogen, V-01 by Livzon Mabpharm Inc, Recombinant SARS-CoV-2 Vaccine (CHO Cell) by National Vaccine and Serum Institute, ReCOV by Jiangsu Rec-Biotechnology Co Ltd, 5-268019 by Shionogi, SCTV01C by Sinocelltech, UB-612 by COVAXX, GBP510 by SK Bioscience Co Ltd, CIGB-66 by Center for Genetic Engineering and Biotechnology (CIGB), AKS-452 by University Medical Center Groningen, Recombinant (Sf9 cell) by West China Hospital, MVC-COV1901 by Medigen, and the like.

Examples of DNA vaccines against SARS-CoV-2 virus include AG0302-COVID19 by AnGes, ZyCoV-D by Zydus Cadila, GX-19 by Genexine, INO-4800 by Inovio, and the like.

Examples of virus-like particles (VLP) vaccines against SARS-CoV-2 virus include LYB001 by Yantai Patronus Biotech Co Ltd, Plant-based VLP by Medicago, and the like.

B. Individuals to be Vaccinated

A subject in need of treatment is a subject having or at risk of having an infection (e.g., a subject having or at risk of contracting a viral, bacterial, fungal, or protozoal infection. The methods are particularly suited for those at risk of exposure to one or more respiratory pathogens such as severe acute respiratory syndrome (SARS) virus and influenza virus. The methods effectively induce mucosal immunity including tissue-resident memory T and B cells. In some embodiments, the methods provide protective mucosal and systemic immunity against one or more antigens from an Orthomyxoviridae (e.g., Influenza virus A and B and C), or Coronaviridae (e.g., Coronavirus, such as severe acute respiratory syndrome (SARS) virus).

A subject having an infection is a subject that has been exposed to an infectious microorganism and has acute or chronic detectable levels of the microorganism in his/her body or has signs and symptoms of the infectious microorganism. Methods of assessing and detecting infections in a subject are known by those of ordinary skill in the art. A subject at risk of having an infection is a subject that may be expected to come in contact with an infectious microorganism. Examples of such subjects are medical workers or those traveling to parts of the world where the incidence of infection is high. Therefore, in some embodiments, the subject is an individual who has one or more risk factors including being a medical worker, living in or traveling to parts of the world where the incidence of infection is high, or who has a close contact with an infected individual, for example, is co-habiting or working with one or more infected individuals. In some embodiments, the subject is at an elevated risk of an infection because the subject has one or more physiological risk factors to have an infection. Examples of risk factors to have an infection include, for example, immunosuppression, immunocompromised, age, trauma, burns (e.g., thermal burns), surgery, foreign bodies, cancer, newborns especially newborns born prematurely. Therefore, in some embodiments, the subject is an individual who has one or more risk factors including immunosuppression, immunocompromised, old age, trauma, burns (e.g., thermal burns), surgery, foreign bodies, cancer, newborns and newborns born prematurely.

The degree of risk of an infection depends on the multitude and the severity or the magnitude of the risk factors that the subject has. Risk charts and prediction algorithms are available for assessing the risk of an infection in a subject based on the presence and severity of risk factors. Other methods of assessing the risk of an infection in a subject are known by those of ordinary skill in the art. In some embodiments, the subject who is at an elevated risk of an infection may be an apparently healthy subject. An apparently healthy subject is a subject who has no signs or symptoms of disease.

1. Diseases to be Prevented

The methods provide protective immunity against infectious diseases. In particular, the methods provide protective immunity against one or more respiratory diseases. The diseases for which protective immunity is provided are determined by the antigen that is provided within the compositions for administration.

In preferred embodiments, the methods vaccinate a subject against infection with a respiratory viral disease, such as infection with a coronavirus associated with the development of severe acute respiratory syndrome (SARS-Cov-2), or an influenza virus.

Where there are multiple pathogenic strains of a pathogen that share one or more of the same antigenic epitopes, or that can be recognized by the same cross-protective immune surveillance molecules in the subject, the methods can provide protective immunity to more than one pathogen in the subject following a single administration of the particle composition to the subject. For example, in some embodiments, the administration of a single antigen species to a subject according to the methods can induce or stimulate protective immunity to a multiplicity of pathogens, or to a multiplicity of strains of a pathogen in the subject. In an exemplary embodiment, the administration of a single SARS coronavirus antigen species to the subject induces or stimulates protective immunity to a multiplicity of SARS-CoV-2 coronaviruses in the subject. In another embodiment, the administration of a single influenza virus antigen species to the subject induces or stimulates protective immunity to a multiplicity of influenza viruses in the subject.

a. Respiratory Infections

In preferred embodiments, the methods prevent infection by respiratory viruses selected from orthomyxoviruses, paramyxoviruses, coronaviruses, adenoviruses, herpesviruses, and human bocaviruses. Exemplary viruses associated with respiratory infections include influenza viruses, parainfluenza viruses, measles viruses, mumps viruses, and respiratory syncytial virus, human metapneumovirus, severe acute respiratory syndrome (SARS) Coronavirus 2, herpes simplex virus (HSV), cytomegalovirus (CMV), Epstein-Barr virus (EBV), and varicella-zoster virus (VZV).

i. SARS

In some embodiments, the methods prevent one or more symptoms of coronavirus infection in the subject. For example, the methods reduce or prevent one or more symptoms or physiological markers of severe acquired respiratory syndrome (SARS) in a subject. In some embodiments, the methods prevent infection by the causative viral disease COVID-19 in a subject. Exemplary symptoms of COVID-19 include cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. Therefore, in some embodiments, the methods prevent or reduce one or more of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

Thus, in some embodiments, the virus comprises a coronavirus. In certain embodiments, the coronavirus comprises at least one of an Alphacoronavirus, a Betacoronavirus, a Gammacoronavirus, and a Deltacoronavirus.

In certain embodiments, the coronavirus is an Alphacoronavirus, such as but not limited to Alphacoronavirus 1, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, and/or Scotophilus bat coronavirus 512. In certain embodiments, the coronavirus is a Betacoronavirus, such as but not limited to Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), and/or Tylonycteris bat coronavirus HKU4. In certain embodiments, the coronavirus is a severe acute respiratory syndrome-related coronavirus (SARS-CoV, SARS-CoV-2). In certain embodiments, the coronavirus is a severe acute respiratory syndrome-related coronavirus, SARS-CoV-2. In certain embodiments, the oronavirus is a Gammacoronavirus, such as but not limited to Avian coronavirus and/or Beluga whale coronavirus SW1. In certain embodiments, the coronavirus is a Deltacoronavirus, such as but not limited to Bulbul coronavirus HKU11 and/or Porcine coronavirus HKU15.

In some embodiments, the coronavirus includes human coronavirus OC43 (HCoV-OC43), human coronavirus HKU1 (HCoV-HKU1), human coronavirus 229E (HCoV-229E), human coronavirus NL63 (HCoV-NL63), severe acute respiratory syndrome coronavirus (SARS-CoV), Middle East respiratory syndrome-related coronavirus (MERS-CoV), severe acute respiratory syndrome coronavirus 2 (SARS-CoV-2), or any other coronaviruses that are capable of infecting human and/or other mammalian subjects.

In certain embodiments, the coronavirus includes at least one of MERS-CoV, SARS-CoV, and SARS-CoV-2.

In certain embodiments, the SARS-CoV-2 includes at least one variant selected from B.1.1.7 (Alpha), B.1.351 (Beta), P.1 (Gamma), B.1.617.2 (Delta), B.1.429/B.1.427 (Epsilon), B.1.617.1 (Kappa), B.1.525 (Eta), B.1.526 (Iota), P.3 (Theta), P.2 (Zeta), and B.1.1.529 (Omicron).

In certain embodiments, the SARS-CoV-2 includes at least one variant selected from A.1-A.6, B.3-B.7, B.9, B.10, B.13-B.16, B.2, B.1 lineage, P.1, P.2, P.3, and R.1.

In certain embodiments, the B.1 lineage includes at least one of (including, but not limited to, B.1, B.1.1, B.1.1.7, B.1.1.7 with E484K, B.1.2, B.1.5-B.1.72, B.1.9, B.1.13, B.1.22, B.1.26, B.1.37, B.1.3-B.1.66, B.1.177, B.1.243, B.1.313, B.1.351, B.1.427, B.1.429, B.1.525, B.1.526, B.1.526.1, B.1.526.2, B.1.617, B.1.617.1, B.1.617.2, B.1.617.3, B.1.619, B.1.620, and B.1.621.

ii. Influenza

In some embodiments, the methods prevent one or more symptoms of influenza virus infection (flu) in the subject. For example, the methods prevent or reduce one or more symptoms or physiological markers of flu in a subject. In some embodiments, the methods prevent infection by one or more influenza viruses, or prevent or reduce one or more symptoms of influenza. Exemplary symptoms of flu include cough, fatigue, fever, body aches, headache, sore throat, vomiting, diarrhea, cytokine storm, skin changes, chest pain and shortness of breath. Therefore, in some embodiments, the methods prevent or reduce one or more of cough, fatigue, fever, body aches, headache, sore throat, chest pain and shortness of breath.

b. Other Diseases

In some embodiments, the methods vaccinate a subject against one or more other infectious diseases, for example, viral diseases such as HIV/AIDS, AIDS Related Complex, Chickenpox (Varicella), Common cold, Cytomegalovirus Infection, Colorado tick fever, Dengue fever, Ebola hemorrhagic fever, Epidemic parotitis, Flu, Hand, foot and mouth disease, Hepatitis, Herpes simplex, Herpes zoster, HPV, Lassa fever, Measles, Marburg hemorrhagic fever, Infectious mononucleosis, Mumps, Poliomyelitis, Progressive multifocal leukoencephalopathy, Rabies, Rubella, Smallpox (Variola), Viral encephalitis, Viral gastroenteritis, Viral meningitis, Viral pneumonia, West Nile disease or Yellow fever; or Bacterial diseases such as Anthrax, Bacterial Meningitis, Brucellosis, Bubonic plague, Campylobacteriosis, Cat Scratch Disease, Cholera, Diphtheria, Epidemic Typhus, Gonorrhea, Hansen's Disease, Legionellosis, Leprosy, Leptospirosis, Listeriosis, Lyme Disease, Melioidosis, MRSA infection, Nocardiosis, Pertussis, Pneumococcal pneumonia, Psittacosis, Q fever, Rocky Mountain Spotted Fever or RMSF, Salmonellosis, Scarlet Fever, Shigellosis, Syphilis, Tetanus, Trachoma, Tuberculosis, Tularemia, Typhoid Fever, Typhus, Whooping Cough; Parasitic diseases such as African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, and Trypanosomiasis.

C. Routes of Administration

The methods can be used to deliver antigen, for example, in the form of polynucleotides to cells in vivo. It has been discovered that the polymers are more efficient and/or less toxic for systemic in vivo transfection of polynucleotides than alternative transfection reagents including LIPOFECTAMINE 2000, PEI, and even other PMSCs. Therefore, in some embodiments, the cell specific particles including a therapeutic polynucleotide are administered systemically in vivo to prevent a disease, for example severe acute respiratory syndrome coronavirus 2 (SARS-CoV-2) and cancer.

The compositions are generally administered to a subject in an effective amount. As used herein the term “effective amount” means a dosage sufficient to inhibit, or prevent one or more infections, or symptoms of a disease or to otherwise provide a desired pharmacologic and/or physiologic effect. The precise dosage will vary according to a variety of factors such as subject-dependent variables (e.g., age, immune system health, etc.), the disease against which immunity is desired, and the treatment being affected.

The pharmaceutical compositions can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some embodiments, the compositions are administered via a systemic or a mucosal route, for example, by intravenous or intraperitoneal administration, in an amount effective for delivery of the compositions to targeted cells.

In certain embodiments, the compositions are administered locally, for example by injection directly into a site to be treated. In some embodiments, the compositions are injected or otherwise administered directly to one or more tumors. Typically, local injection causes an increased localized concentration of the compositions which is greater than that which can be achieved by systemic administration. In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can affect a sustained release of the particles to the immediate area of the implant.

The particles can be provided to the cells either directly, such as by contacting it with the cell, or indirectly, such as through the action of any biological process. For example, the particles can be formulated in a physiologically acceptable carrier or vehicle, and injected into a tissue or fluid surrounding the cell. The particles can cross the cell membrane by simple diffusion, endocytosis, or by any active or passive transport mechanism.

Intranasal vaccine will generate a robust population of lung T_{RM}. Modes of vaccine delivery play a critical role in the generation of T_{RM}. Different modes of vaccine delivery can generate similar levels of systemic antibody and T_{CM}, but vastly different levels of mucosal antibody and T_{RM}. The presence or absence of T_{RM} is reflected in resistance to infection, and demonstrates clearly that simply sampling blood for antibody titers and/or memory T cell abundance and function as a surrogate for protective immunity falls far short of this goal and may even be misleading.

1. Administration Via Mucosal Tissue

The particles are useful in delivery of antigens. The particles are particularly suited for administration to the nasal or pulmonary system, or administered to a mucosal surface (vaginal, rectal, buccal, sublingual). The particles may be administered as a dry powder, as an aqueous suspension (in water, saline, buffered saline, etc.), in a hydrogel, organogel, or liposome, in capsules, tablets, troches, or other standard pharmaceutical excipient. [000200]

Suitable compositions and dosage forms include, for example, tablets, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, or aerosolized formulations for inhalation, compositions and formulations for intravesical administration and the like. It should be understood that the formulations and compositions that would be useful in the instant specification are not limited to the particular formulations and compositions that are described herein.

The particular polynucleotide delivered by the particle can be selected by one of skill in the art depending on the condition or disease to be treated. The polynucleotide can be, for example, a gene or cDNA of interest, mRNA, a functional nucleic acid such as an inhibitory RNA, a tRNA, an rRNA, or an expression vector encoding a gene or cDNA of interest, a functional nucleic acid a tRNA, or an rRNA. In some embodiments two or more polynucleotides are administered in combination.

D. Administration/Dosing

In clinical settings, delivery systems for the compositions described herein can be introduced into a subject by any of a number of methods, each of which is familiar in the art. For instance, a pharmaceutical formulation of the composition can be administered by inhalation or systemically, e.g., by intravenous injection.

The regimen of administration may affect what constitutes an effective amount. The therapeutic formulations may be administered to the subject either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the composition of the instant specification to a subject, preferably a mammal, more preferably a human, may be carried out using known procedures, at dosages and for periods of time effective to treat a disease or condition in the subject. An effective amount of the composition necessary to achieve a therapeutic effect may vary according to factors such as the time of administration; the duration of administration; other drugs, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the composition without undue experimentation. Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The therapeutically effective amount or dose of an antigen of the instant specification depends on the age, sex and weight of the patient, the current medical condition of the patient and the progression of a disease or disorder contemplated herein in the patient being treated. The skilled artisan is able to determine appropriate dosages depending on these and other factors.

A suitable dose of an antigen of the instant specification may be in the range of from about 0.001 mg to about 5,000 mg per day, such as from about 0.01 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

Actual dosage levels of the cells in the pharmaceutical formulations of the instant specification may be varied so as to obtain an amount of the composition that are effective to achieve the desired therapeutic response for a particular subject, composition, and mode of administration, without being toxic to the subject.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized.

E. Controls

The effect of the polynucleotides-based vaccines associated or encapsulated within poly(amine-co-ester) polymers in the form of micellular particles or solid polymeric nanoparticles in inducing a mucosal immune response and/or a long lasting and protective mucosal immunity specific to the vaccinating antigen in a subject can be compared to a control. In some embodiments, the control is one having had a priming immunogenic composition and/or a boosting immunogenic composition via non-mucosal route, or priming and boosting both via intramuscular route. In some embodiments, the control is one having had a priming immunogenic composition and/or a boosting immunogenic composition without the use of poly(amine-co-ester) polymers. In a further embodiment, the control is one having had a priming immunogenic composition and a boosting immunogenic composition, neither of which uses of poly(amine-co-ester) polymers as a vaccine carrier.

Suitable controls are known in the art and include, for example, an untreated subject, or a placebo-treated subject. A typical control is a comparison of a condition or symptom of a subject prior to and after administration of the composition. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

### VI. Kits

Kits or packs that supply the elements necessary for inducing mucosal immunity, including, but not limited to, protective immunity against one or more pathogenic antigens are also disclosed. The kits include a first composition used to “prime” the subjects immune system and a second composition to “spike” the subject.

Accordingly, in some aspects, the present invention is directed to a kit for eliciting an immune response in/vaccinating a subject against an infection by a virus. In some embodiments, the virus is one that causes a respiratory infection. In some embodiments, the virus is a coronavirus, an influenza virus, a herpes simplex virus, or any combinations thereof.

In some embodiments, the viral infection is the same as or similar to those as described elsewhere herein.

Thus, in some embodiments, the kit contains a first composition for priming an immune system of the subject; and a second composition for boosting an immunization following vaccination with the first composition.

In some embodiments, the first composition is the same as or similar to those used in the “priming” step as described elsewhere herein.

In some embodiments, the second composition is the same as or similar to those used in the “spiking” step as described elsewhere herein.

In accordance with one embodiment a kit is provided comprising the polymers, optionally a coating, for example a target specific coating, and an antigenic polypeptide or a polynucleotide encoding the antigen, form a complex which can be used to deliver the antigen to a target cell. The particle can be further mixed with the coating to provide cell-type or cell-state specific tropism.

In one embodiment, the kits include a priming immunogenic composition including one or more messenger ribonucleic acids encoding an exogenous antigen in an intramuscular or intradermal formulation suitable as a first composition, and the kit can include as the second composition, a composition containing a viral antigen or mRNA encoding a viral antigen, preferably, encapsulated in PACE particles. The kits include a immunogenic composition including particles (such as microparticles, and/or nanoparticles) formed from poly(amine-co-ester)s or poly(amine-co-amide)s having encapsulated therein one or more messenger ribonucleic acids encoding the same exogenous antigen in an intranasal formulation, which can be the first and/or the second composition, in some embodiments. In another embodiment, the priming vaccine composition and the boosting vaccine composition are packaged in separate containers.

The individual components of the kits can be packaged in a variety of containers, e.g., vials, tubes, microtiter well plates, bottles, and the like. Other reagents can be included in separate containers and provided with the kit; e.g., positive control samples, negative control samples, buffers, cell culture media, etc. Preferably, the kits will also include instructions for use.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided.

### Example 1

Materials and Methods

Cell and Virus

As reported in Israelow et al. (Sci Immunol 6, eabl4509 (2021)), Gartlan et al. (Front Immunol 13, 882972 (2022)), and Sanchez et al., (Science Immunology 6, eabi8635 (2021)), Vero E6 cells overexpressing angiotensin-converting enzyme 2 (ACE2) and TMPRSS2 [kindly provided by B. Graham at the National Institutes of Health Vaccine Research Center (NIH-VRC)] were cultured in Dulbecco's modified Eagle medium (DMEM) supplemented with 1% sodium pyruvate and 5% fetal bovine serum (FBS) at 37° C. and 5% CO₂. SARS-CoV-2 isolate hCOV-19/USAWA1/2020 (NR-52281) was obtained from BEI Resources and was amplified in VeroE6 cells overexpressing ACE2 and TMPRSS2. Cells were infected at a multiplicity of infection 0.01 for 2 to 3 days to generate a working stock, and after incubation, the supernatant was clarified by means of centrifugation (5 min, 500 g) and filtered through a 0.45-mm filter and stored at −80° C. Viral titers were measured with standard plaque assay by using Vero E6 cells overexpressing hACE2 and TMPRSS2.

Animals

B6.Cg-Tg(K18-ACE2)2Prlmn/J (K18-hACE2) mice (stock no. 034860) were purchased from the Jackson Laboratory and subsequently bred and housed at Yale University. Eight- to twelve-week-old female mice were used for immunization experiments. Male Syrian hamsters (strain, HSdHan:AURA; stock no. 089) were purchased from Envigo, and vaccination began at 12 weeks of age. All procedures used in this study (such as sex matching and age matching) complied with federal guidelines and the institutional policies of the Yale School of Medicine Animal Care and Use Committee. To reduce the overall number of experimental animals used and to be consistent with our institutional animal use policy, control data points are shared among some FIGs. when applicable and noted in figure legends. Sample sizes for animal experiments were determined empirically on the basis of previously published work in the field with similar experimental paradigms to provide sufficient statistical power for assessing biological effects of interest. No statistical methods were used to predetermine the sample size. Age- and sex-matched animals were randomly assigned to experimental groups at the beginning of the experiment. Investigators were not blinded, except for pathological analysis, because no subjective measurements were performed.

SARS-CoV-2 Infection

Mice were anesthetized by using 30% v/v iso-flurane diluted in propylene glycol. Using a pipette, 50 ml containing 6×104 plaque-forming units (PFU) SARS-CoV-2 was delivered intra-nasally. Hamsters were anesthetized by using 30% v/v isoflurane diluted in propylene glycol and administered 6×10 PFU SARS-CoV-2 intranasally in 100 ml.

mRNA Extraction from Comirnaty (BNT162b2) mRNA-LNP

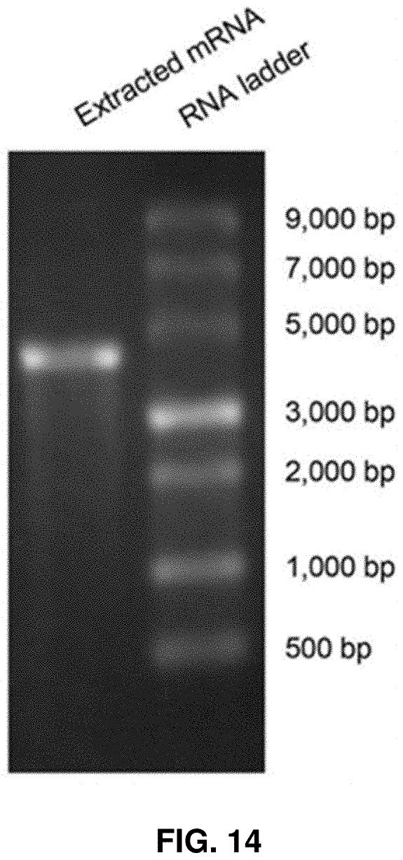

mRNA was extracted from the vaccine formulation with a TRIzol-chloroform separation method as previously described (41). Briefly, aliquots of vaccine were dissolved in TRIzol LS (Thermo Fisher Scientific) at 1:6.6 vaccine to TRIzol volume ratio. After a 15-min incubation (37° C., shaking), 0.2 ml of chloroform was added per 1 ml of TRIzol. The solution was shaken vigorously for 1 min and then incubated at room temperature for 3 min. The solution was centrifuged at 12,000 g for 8 min at 4° C. The aqueous layer containing the isolated mRNA was further purified with a RNeasy Maxi Kit purchased from Qiagen (Germantown, MD, USA) following the manufacturers protocol. The RNA was eluted from the column on the final step with sodium acetate buffer (25 mM, pH 5.8) warmed to 37° C. Extracted mRNA was analyzed for concentration and purity with NanoDrop measurements of the absorbance at 260, 280, and 230 nm, with purity being assessed as A260/A280>2 and A260/A230>2. Agarose gel electrophoresis was used to determine the length and verify that the mRNA remained intact. Extracted mRNA containing 1:100 SYBR Safe stain (Thermo Fisher Scientific) was loaded onto a 1% agarose gel and run at 75 V with tris-acetate-EDTA buffer containing 1:5000 SYBR Safe stain (FIG. 14).

ACE Polyplex Formulation and Characterization

PACE polymers were synthesized and characterized as previously described. All polyplexes were formulated at a 50:1 weight ratio of polymer to mRNA. PACE polymers were dissolved at 100 mg/ml overnight in dimethyl sulfoxide (37° C., shaking). Before polyplex fabrication, an optimal PACE polymer blend was produced by mixing solutions of PACE polymers containing an end-group modification and a polyethylene glycol tail. mRNA and polymer were diluted into equal volumes of sodium acetate buffer (25 mM, pH 5.8). The polymer dilution was then vortexed for 15 s, mixed with the mRNA dilution, and vortexed for an additional 25 s. Polyplexes were incubated at room temperature for 10 min before use.

Vaccination

Used vials of Comirnaty vaccine were acquired from Yale Health pharmacy within 24 hours of opening and stored at 4° C. Vials contained residual vaccine (diluted to 100 mg/ml per manufacturer's instructions), which was removed with spinal syringe and pooled. Pooled residual vaccine was aliquoted and stored at −80° C. Mice were anesthetized by using a mixture of ketamine (50 mg per kilogram of body weight) and xylazine (5 mg per kilogram of body weight) and injected intraperitoneally. Vaccine was diluted in sterile phosphate-buffered saline (PBS) and 10 or 20 ml was injected into the left quadriceps muscle with a 31G syringe for a final dose of 1 or 0.05 mg as indicated. Similarly, hamsters were administered 0.5 mg diluted in 20 ml by means of 31G syringe in the left quadriceps muscle. For IN vaccination, SARS-CoV-2-stabilized spike (ACRO biosystems, SPN-052H9) or SARS-CoV-1 spike (ACRO biosystems, SPN-S52H6) was re-constituted in sterile endotoxin-free water ac-cording to the manufacturer's protocol and then diluted in sterile PBS and stored at −80° C. Mice or hamsters were anesthetized by using isoflurane and administered 1 or 5 mg (as indicated) in 50 ml (25 ml where indicated) through the IN route. For IN mRNA-PACE, 50 ml of polyplexes in solution was administered at the indicated dose.

Viral Titer Analysis

Viral titer analysis was performed as previously described, with modifications noted and summarized here. Mice were euthanized in 100% isoflurane at indicated time points. Approximately half of the total lung (right lobes) or nasal turbinate was homogenized in a bead homogenizer tube containing 1 ml of PBS supplemented with 2% FBS and 2% antibiotics/antimycotics (Gibco) and stored at −80° C. Nasal turbinate and lung homogenates were clarified of debris by centrifugation (10 min, 3100 g). Daily oral swabs (Pruitan PurFlock Ultra 25-3206-U) were performed on hamsters and stored in 1 ml of DMEM with 2% FBS and 2% antibiotics/antimycotics (Gibco) and stored at −80° C. To determine infectious SARS-CoV-2 titers, plaque assay was performed by using ACE2- and TMPRSS2-overexpressing Vero E6 cells. Plaques were resolved by means of formalin fixation 40 to 42 hours after infection, followed by staining with crystal violet and rinsing with water for plaque visualization.

SARS-CoV-2-Specific Antibody Measurements

Enzyme-linked immunosorbent assays (ELISAs) were performed as previously described (Isralow, et al., J. Exp. Med., 217:e20201241 (2020), Armat, et al., Nat Med., 26:1033-36 (2020)), with modifications noted and summarized here. Ninety-six-well MaxiSorp plates (Thermo Scientific 442404) were coated with recombinant SARS-CoV-2 S1 protein (ACRO Biosystems S1N-052H3) or SARS-CoV-1 S1 protein (ACRO Biosystems S1N-S52H5). After overnight incubation at 4° C., plates were replaced with blocking solution (PBS with 0.1% Tween-20, and 5% milk powder) and incubated for 1 to 2 hours at room temperature. Serum or BALF was diluted in dilution solution (PBS with 0.1% Tween-20 and 2% milk powder) and added to plates for 2 hours at room temperature. Plates were washed five times with PBS-T (PBS with 0.05% Tween-20) by using an automatic plate washer (250 ml per cycle), and 50 ml of horseradish peroxidase (HRP) anti-mouse IgG (Cell Signaling Technology 7076; 1:3000), HRP anti-mouse IgA (South-ern Biotech 1040-05; 1:1000), HRP anti-hamster IgG (Southern Biotech 6060-05; 1:1000), or rabbit anti-hamster IgA HRP (Brookwood Bio-medical, sab3003a, 1:250 100 mg/ml) diluted in dilution solution was added to each well. After 1 hour of incubation at room temperature (over-night at 4° C. for hamster IgA), plates were washed three times with PBS-T by using an automatic plate washer. Fifty μl of TMB Substrate Reagent Set (BD Biosciences 555214) was added to plates. To terminate the reaction, another 50 ml of 2N sulfuric acid was added after 15 min of substrate development. Plates were then recorded at wavelengths of 450 and 570 nm, and the difference was reported as AUC.

Immunohistochemistry and Pathological Analysis

Yale Pathology Tissue Services (YPTS) performed embedding, sectioning, and hematoxylin and eosin (H&E) staining of lung tissue. A pulmonary pathologist reviewed the slides blinded and identified immune cell infiltration and other related pathologies. Mouse lung scores of 1 to 4 were characterized as follows: 1, mild patchy mononuclear infiltrate, parenchymal and perivascular, with variably reactive pneumocytes and stromal reaction; 2, moderate patchy mononuclear infiltrate, parenchymal and perivascular, with variably reactive pneumocytes and stromal reaction; 3, mild, dense mixed infiltrate, including mononuclear cells and granulocytes and neutrophils; and 4, moderate, dense mixed infiltrate, including mononuclear cells and granulocytes and neutrophils. Hamster lung scores of 0 to 4 were characterized as follows: 0, normal; 1, very focal injury, inflammation, and repair; 2, multifocal repair; and 4, multifocal repair with necrosis.

Intravascular Labeling, Cell Isolation, and Flow Cytometry

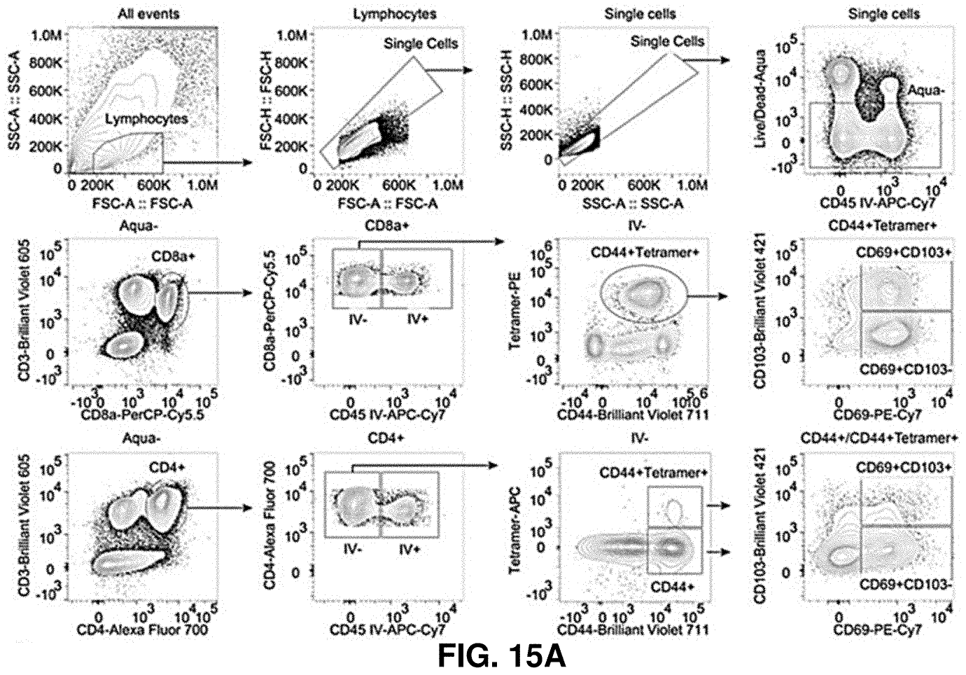

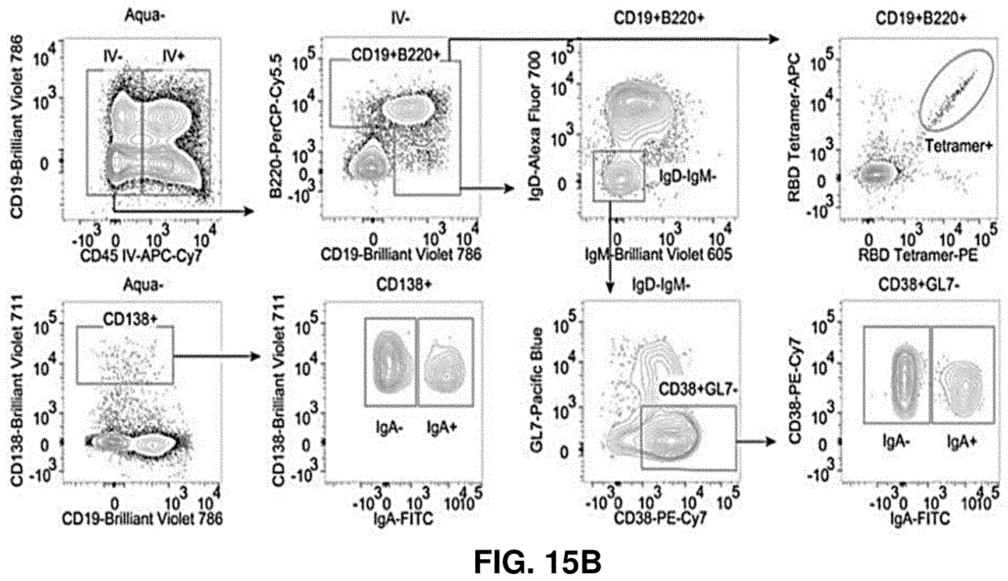

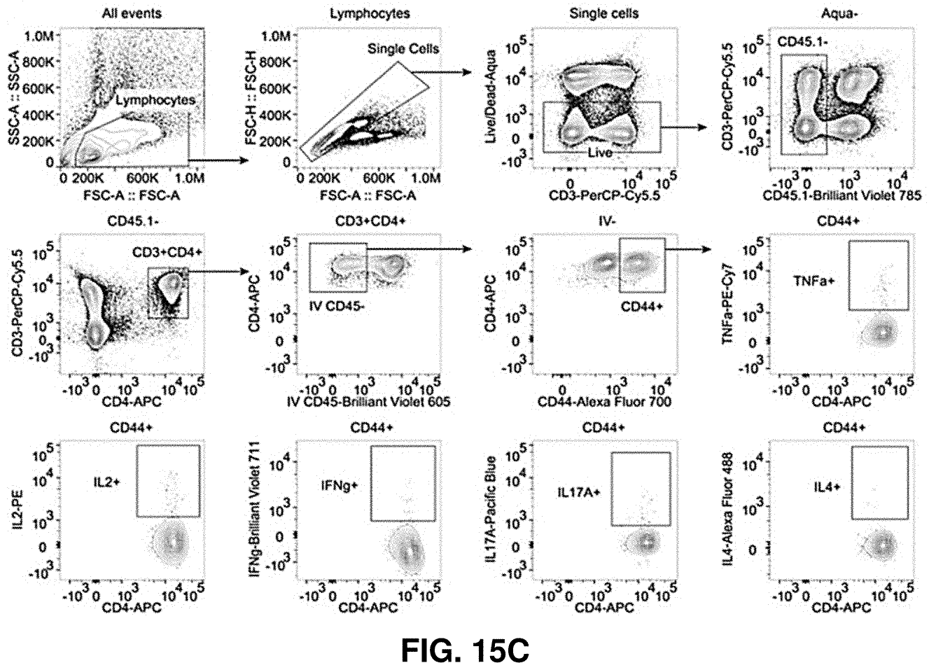

To discriminate circulating from extravascular immune cells, mice were anesthetized with 30% isoflurane and injected intravenously with 2 mg of APC/Fire 750-labeled anti-CD45 Ab. After 3 min of labeling, mice were euthanized. Tissues were harvested and analyzed as previously described (39). Briefly, lungs and nasal turbinates were minced with scissors, incubated in a digestion cocktail containing collagenase A (Roche) and DNase I (Sigma-Aldrich) in RPMI at 37° C. for 45 min, and dissociated through a 70-mm filter. Airway-resident immune cells were collected by centrifuging BALF at 600 g for 5 min at 4° C., after which cell pellets were used for flow cytometry, and supernatants were used for antibody analysis. Cells were treated with ammonium-chloride-potassium (ACK) buffer to lyse red blood cells and then washed once with PBS. Single-cell suspensions were incubated with Fixable Aqua cell viability dye (Invitrogen L34957) and anti-mouse CD16/CD32 Fc Block (BD Biosciences 553141) for 30 min at 4° C. Cells were washed once with PBS before surface staining. For T cell analysis, cells were first stained with APC-labeled SARS-CoV-2 S 62-76 MHC class II tetramer [I-A(b)] for 60 min at RT. Cells were washed once with PBS and then stained with anti-CD103, anti-CD3, anti-CD44, anti-CD62L, anti-CD8a, anti-CD69, anti-CD183 (CXCR3), anti-CD4, and PE-SARS-CoV-2 S 539-546 MHC class I tetramer [H-2K(b)] for 30 min at 4° C. For B cell analysis, cells were stained with ant-GL7, anti-IgM, anti-CD138, anti-CD19, anti-IgA, anti-B220, PE-SARS-CoV-2 RBD tetramer, anti-CD38, APC-SARS-CoV-2 RBD tetramer, and anti-IgD for 30 min at 4° C. Cells were washed with PBS once, followed by 4% paraformaldehyde fixation for 45 min at 4° C. Flow cytometry data were acquired on an At-tune NxT Flow Cytometer and analyzed by use of FlowJo Software (10.5.3; Tree Star). Gating strategy is provided in FIGS. 15A-15C, and detailed antibody information is provided in the Table 1 below.

TABLE S1
Specificities, conjugated fluorophores, clone numbers, catalogue numbers, vendors,
dilutions, and final concentrations for flow cytometry reagents used in this study.

| | | | | | Final Conc. |
|---|---|---|---|---|---|
| Reagent | Fluorophore | Clone# | Cat# | Vendor | (μg/ml) |
| Anti-B220 | PerCP/Cy5.5 | RA3-6B2 | 103236 | BioLegend | 63.9 |
| Anti-CD3 | PerCp/Cy5.5 | 17A2 | 100218 | BioLegend | 36.1 |
| Anti-CD3 | BV605 | 17A2 | 100237 | BioLegend | 63.9 |
| Anti-CD4 | AF700 | GK1.5 | 100430 | BioLegend | 159.7 |
| Anti-CD4 | AF647 | GK1.5 | 100530 | BioLegend | 90.3 |
| Anti-CD8a | PerCP/Cy5.5 | 53-6.7 | 100734 | BioLegend | 63.9 |
| Anti-CD19 | BV785 | 6D5 | 115543 | BioLegend | 5.4 |
| Anti-CD38 | PE/Cy7 | 90 | 102718 | BioLegend | 63.9 |
| Anti-CD44 | AF700 | IM7 | 103026 | BioLegend | 159.7 |
| Anti-CD44 | BV711 | IM7 | 103057 | BioLegend | 63.9 |
| Anti-CD45.1 | BV785 | A20 | 110743 | BioLegend | 36.1 |
| Anti-CD62L | FITC | MEL-14 | 104406 | BioLegend | 159.7 |
| Anti-CD69 | PE/Cy7 | H1.2F3 | 104512 | BioLegend | 36.1 |
| Anti-CD103 | BV421 | 2E7 | 121422 | BioLegend | 36.1 |
| Anti-CD138 | BV711 | 281-2 | 142519 | BioLegend | 63.9 |
| Anti-CXCR3 | APC | CXCR3-173 | 126512 | BioLegend | 63.9 |
| Anti-GL7 | Pacific Blue | GL7 | 144614 | BioLegend | 159.7 |
| Anti-IgA | FITC | N/A | 1040-02 | Southern Biotech | 319.4 |
| Anti-IgD | AF700 | 11-26c.2a | 405730 | BioLegend | 159.7 |
| Anti-IgM | BV605 | RMM-1 | 406523 | BioLegend | 63.9 |
| Anti-IL-2 | PE | JES6-5H4 | 503808 | BioLegend | 36.1 |
| Anti-IL-4 | AF488 | 11B11 | 504109 | BioLegend | 90.3 |
| Anti-IL-17A | BV421 | TC11-18H10.1 | 506926 | BioLegend | 36.1 |
| Anti-TNF-α | PE/Cy7 | MP6-XT22 | 506324 | BioLegend | 36.1 |
| Anti-INF-γ | BV711 | XMG1.2 | 505836 | BioLegend | 36.1 |
| SCV2 Spike | APC | N/A | N/A | NIH Tetramer | 234.7 |
| S62-76 MHC II | | | | Core | |
| tetramer | | | | | |
| SCV2 Spike | PE | N/A | N/A | NIH Tetramer | 469.6 |
| S539-546 MHC I | | | | Core | |
| tetramer | | | | | |
| Anti-CD45 | APC/Fire 750 | 30-F11 | 103154 | BioLegend | 2 μg/mouse |
| Anti-CD45 | BV605 | 30-F11 | 103140 | BioLegend | 2 μg/mouse |

Intracellular Cytokine Staining Assay for Detection of Lung-Resident Spike-Specific CD4 T Cells

After intravascular labeling by using an anti-CD45 Ab at the dose of 2 mg per mouse, lung isolation, and processing, single cells from the lung tissue were first enriched by using a Percoll gradient before spike peptide stimulation. Briefly, total lung cells were first re-suspended in 5 ml of 30% Percoll solution in a 15-ml conical tube, underlaid with 5 ml of 70% Percoll solution, and subject to centrifugation at 1000 g for 20 min at room temperature. After centrifugation, lymphocytes located at the interphase between 30 and 70% Percoll solution were collected, washed once with PBS, and re-suspended in complete RPMI. In a 96-well U-bottom plate, 106 lymphocytes enriched from each lung sample were added, together with spike peptide megapool from SARS-CoV-2 (JPT PM-WCPV-S-1) or SARS-CoV-1 (JPT PM-CVHSA-S-1) at a final working concentration of 1 mg/ml per peptide, 1× Protein Transport Inhibitor Cocktail (eBioscience 00-4980-03), and 106 freshly isolated splenocytes from CD45.1+ mice, with complete RPMI for a final volume of 200 ml. Peptide stimulation was performed for 8 hours at 37° C. After peptide stimulation, cells were incubated at 4° C. with Fc block (BioXCell BE0307) and Aqua cell viability dye (ThermoFisher L34957) for 20 min. Cells were washed once with PBS before surface staining with anti-CD3, anti-CD44, anti-CD4, and anti-CD45.1. After washing with PBS, cells were fixed by using 4% paraformaldehyde for 45 min at 4° C. Cells were then washed and permeabilized with 1× Permeabilization Buffer (eBioscience 00-8333-56) for 10 min at RT. After permeabilization, cells were stained with anti-IL-4, anti-IL-2, anti-tumor necrosis factorα (TNF-α), anti-IL-17A, and anti-interferon-γ (IFN-γ). Cells were washed once with PBS before being acquired on Attune and analyzed by use of FlowJo. Gating strategy is provided in FIGS. 15A-15C, and detailed antibody information is provided in table 1.

SARS-CoV-2 RBD B Cell Tetramer Production and Staining

Recombinant SARS-CoV-2 spike RBD His Biotin Protein, CF (R&D BT10500-050) was incubated at a 4:1 molar ratio with either streptavidin-PE (Prozyme PJRS25) or streptavidin-APC (Prozyme PJ27S) for 30 min at 4° C. Mixture was then purified and concentrated in an Amicon Ultra (50kDA MWCO) spin column and washed 1× with sterile cold PBS. The concentration was determined on a NanoDrop 8000 Spectrophotometer (ThermoFisher ND-8000-GL) by using fluorophore-specific absorbances. Tetramers were then diluted to 1.0 mM in PBS and stored at 4° C. For every 2×10⁷ to 5×10⁷ cells, 1 ml of stock 1.0 mM tetramer was used for staining.

Pseudovirus Production and Neutralization Assay

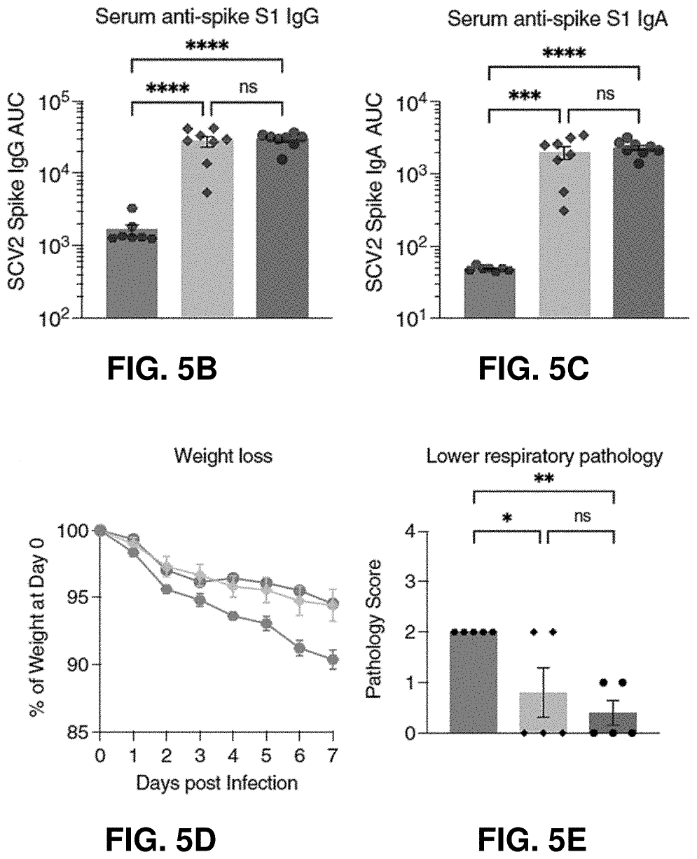

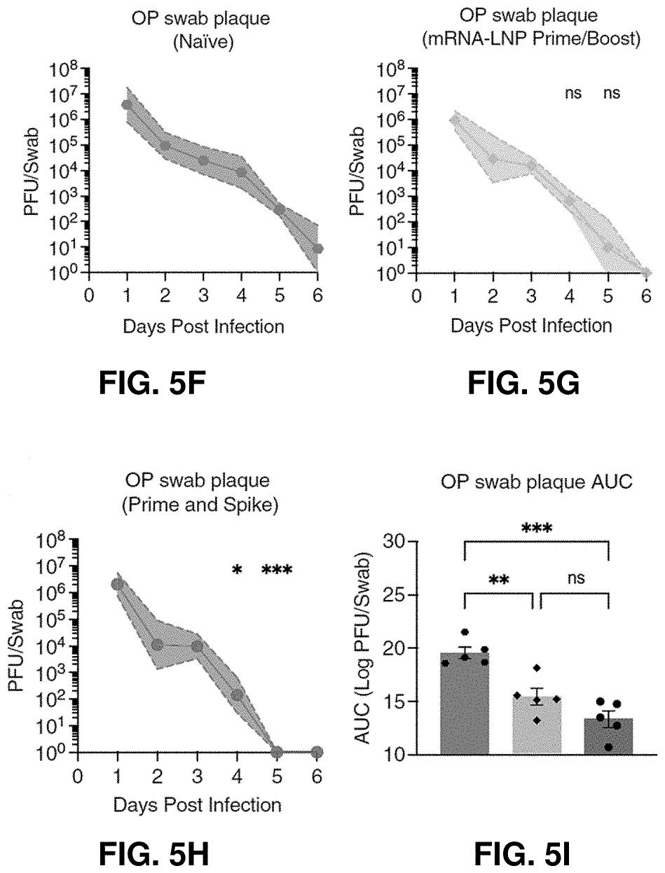

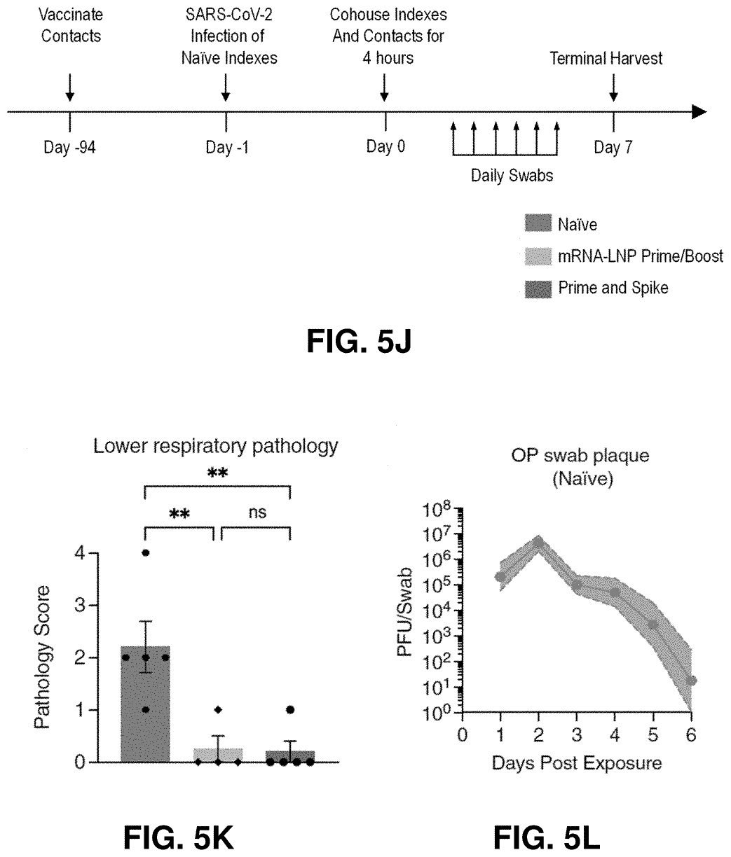

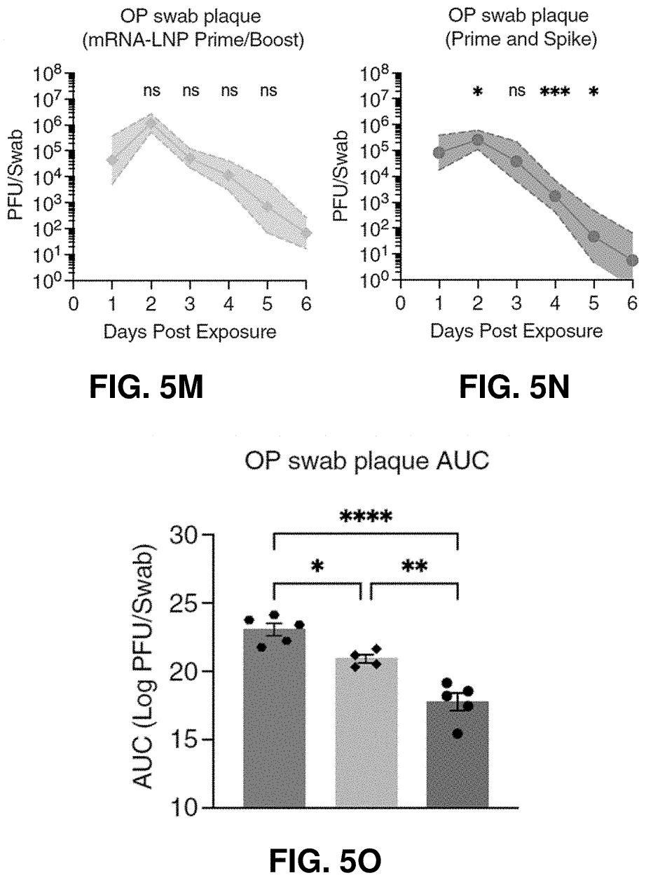

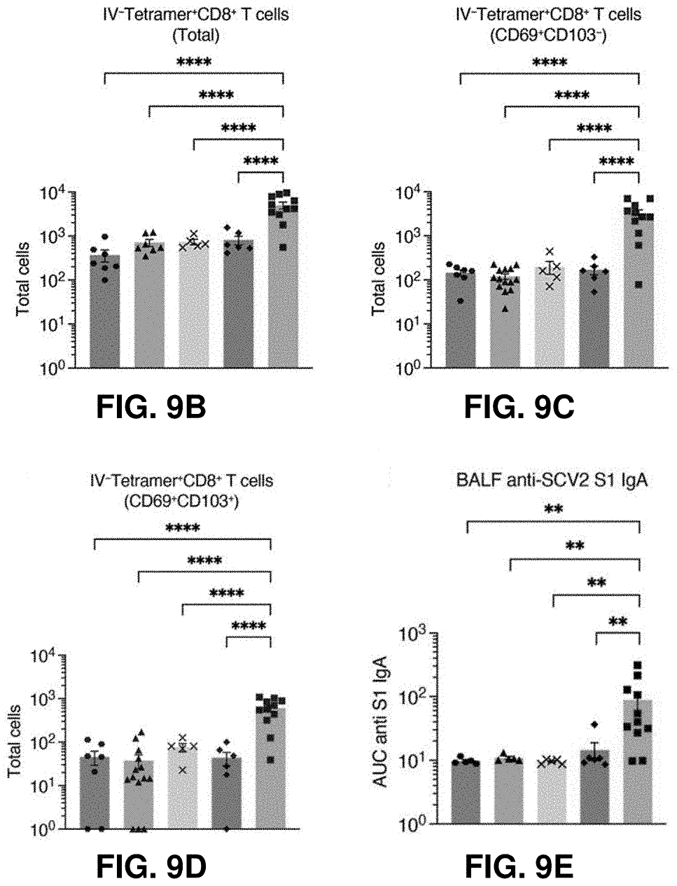

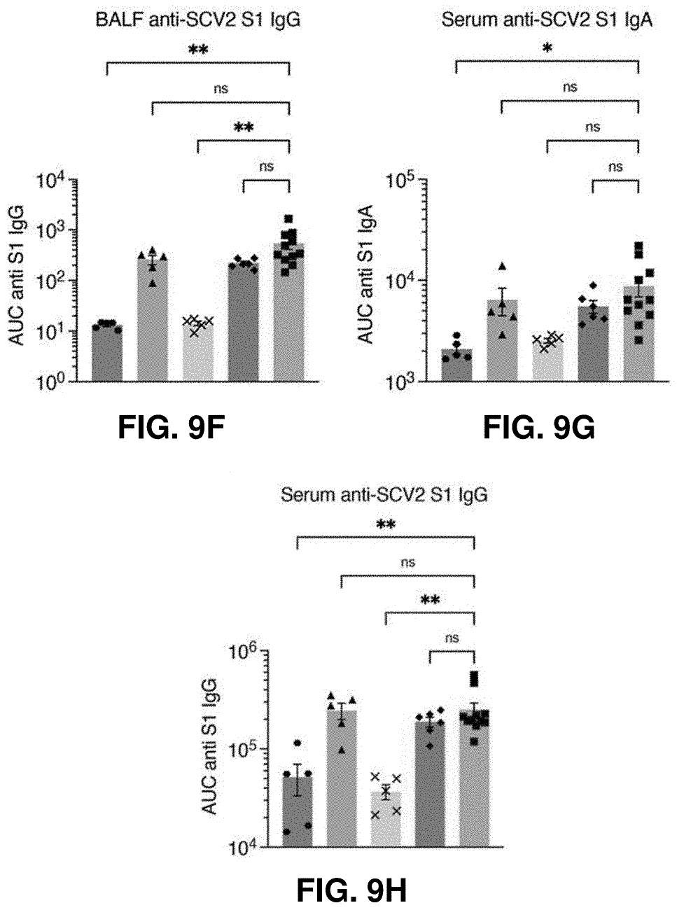

Pseudoviruses were produced as previously described (Israelow, et al., Immunol., 6, eabl4509 (2021)). Spike-encoding plasmid was kindly provided by V. Munster and previously described (45). To perform pseudovirus neutralization assays, Vero E6 overexpressing hACE2 and TMPRSS2 (FIG. 1) or Huh7.5 cell (FIG. 5 and FIG. 9A-H) were plated (3×104) in each well of a white 96-well plate the day before infection. On the day of infection, serum and BALF were heat-inactivated for 30 min at 56° C. Sera shown in FIG. 1 were tested at a starting dilution of 1:50, and BALF samples were tested at a starting dilution of 1:4, both with eight two-fold serial dilutions. Sera shown in FIG. 5 and FIG. 9A-H were tested at a starting dilution of 1:40 with eight threefold serial dilutions. Serial dilutions were mixed 1:1 with indicated pseudo-virus and incubated for 1 hour at 37° C. and 5% CO2. Growth medium was then aspirated from the cells and replaced with 100 ml of serum-virus mixture. Twenty-four hours after infection, the infection-antibody mixture was removed, and plates were flash-frozen at −80° C. Thirty ml of passive lysis buffer (Promega) was added to each well, and plates were incubated for 15 min at room temperature. Thirty ml of Renilla-Glo Luciferase Assay System substrate (Promega) was then added to each well and incubated at room temperature for an additional 15 min. Luminescence was measured on a microplate reader (SpectraMax i3, Molecular Devices). Median inhibitory concentration was calculated with Prism 9 (GraphPad Software) nonlinear regression.

Sequence Alignment

The following amino acid sequences of corona-virus Spike proteins used in alignment were obtained from Uniprot/Genebank: Wuhan (PODTC2), B.1.1.7 (QWE88920.1), B.1.351 (QRN78347.1), B.1.617 (QUD52764.1), B.1.1.28.1 (QRX39425.1), BA.1 (UFO69279.1), BA.2 (UFO69279.1), BA.2.12.1 (UMZ92892.1), BA.4 (UPP14409.1), BA.5 (UOZ45804.1), Khosta (MZ190137.1), Khosta-2 (MZ190138.1), SARS-CoV (AY278489.2), WIV1 (KF367457), and BANAL236 (MZ937003.1). Sequence alignment was performed with MAFFT in JalView (v2.11.2.3).

Results

IN Boosting with Unadjuvanted SARS-CoV-2 Spike Induces Mucosal Humoral Immunity

To assess the potential of IN unadjuvanted subunit vaccine boosting for the development of respiratory mucosal immunity, we decided to harness the strong systemic immunogenicity of mRNA-LNP. We additionally benefited from extensive SARS-CoV-2 spike engineering by using HexaPro, which has been shown to substantially enhance immunogenicity and increase protein stability (Hsieh et al. Science 369, 1501-1505 (2020)).

K18-hACE2 (mice) were vaccinated with 1 μg mRNA-LNP (Pfizer/BioNTech BNT162b2) by means of IM injection (prime), followed 14 days later with IN administration of 1 μg recombinant unadjuvanted spike protein [prime and spike (P&S)]. Mice were euthanized at days 21 or 28 and assessed for mucosal humoral immunity (FIG. 1A).

First, anti-SARS-CoV-2 spike IgG and IgA were assessed in nasal wash (FIGS. 1B and 1C), broncho-alveolar lavage fluid (BALF) (FIGS. 1D and 1E), and serum (FIGS. 1F and 1G). Only mice that received P&S developed high levels of anti-SARS-CoV-2 IgA and IgG in the nasal wash and BALF. Neither IM prime nor IN spike alone was sufficient to develop mucosal antibodies. In the serum, prime alone was sufficient to induce low levels of IgA and IgG. By contrast, P&S led to significant systemic boosting of both anti-spike IgA and IgG. These increases in antibody levels correlated with increases in neutralization titers both in BALF (FIG. 1H) and serum (FIG. 1I). Thus, a single-dose unadjuvanted IN spike alone is not immunogenic, and the induction of a po-tent mucosal and systemic antibody response by unadjuvanted spike requires prior systemic priming, in this case with mRNA-LNP.

Tissue resident memory B cells (B_{RM}) cells in the lungs assist in rapid recall response of antibody-secreting plasma cells upon secondary heterologous challenge in influenza models and may be an important local immune effector in protecting against SARS-CoV-2 (29). Using intravenous (IV) CD45 labeling to differentiate circulating immune cells within lung tissue combined with B cell tetramers specific for the receptor binding domain (RBD) of the spike protein, the data showed that P&S leads to increased antigen-specific B cells with-in lung tissue (IV⁻CD45⁻B220⁺CD19⁺tetramer⁺) (FIG. 1J). The present studies also examined the polyclonal tissue response, which likely represents a more complete set of spike-specific B cells within the lungs. The data showed increases in class-switched antibody-secreting cells (ASCs) (IV⁻CD45⁻CD1^{9+/-}CD138⁺) in lung tissue expressing IgA or IgG (FIGS. 1K and 1L), and increased class-switched B_{RM} cells (IV− CD45⁻B220⁺CD19⁺IgD⁻IgM⁻CD38⁺) expressing IgA or IgG (FIGS. 1M and 1N). Thus, P&S elicits local B cell responses in the lung.

Prime and Spike Induces Mucosal T Cell Immunity

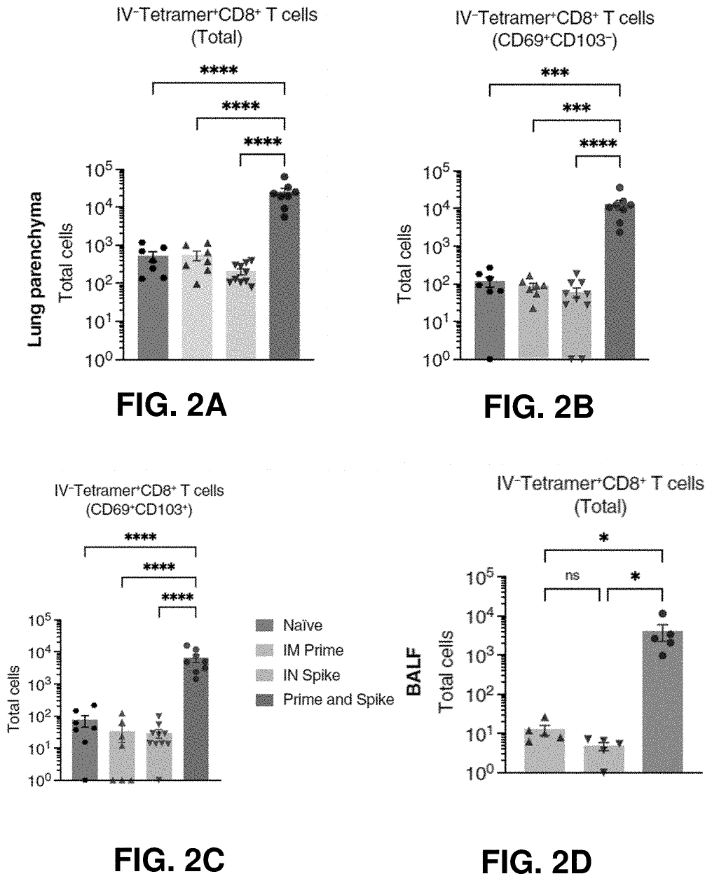

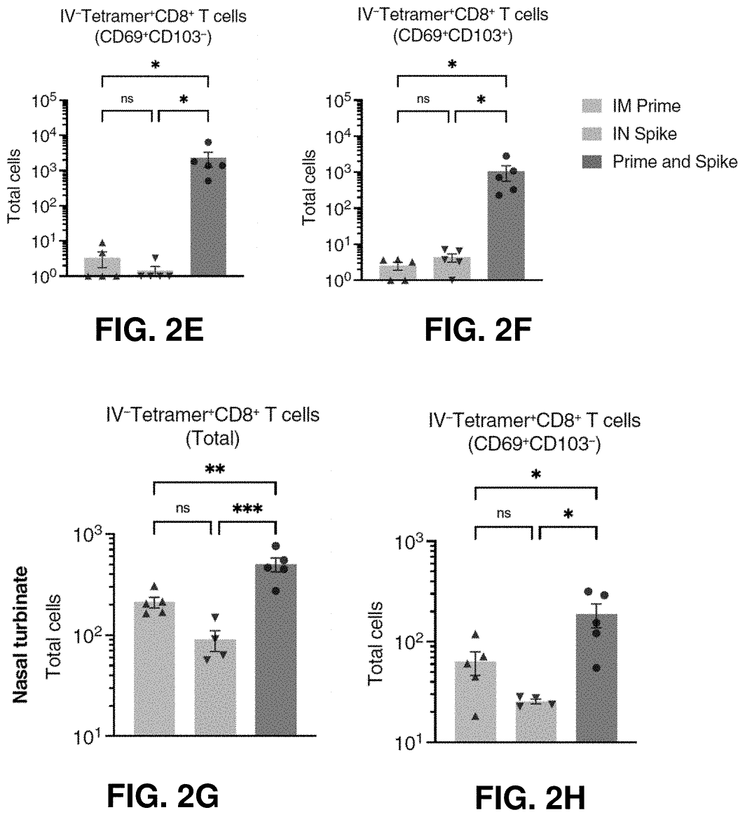

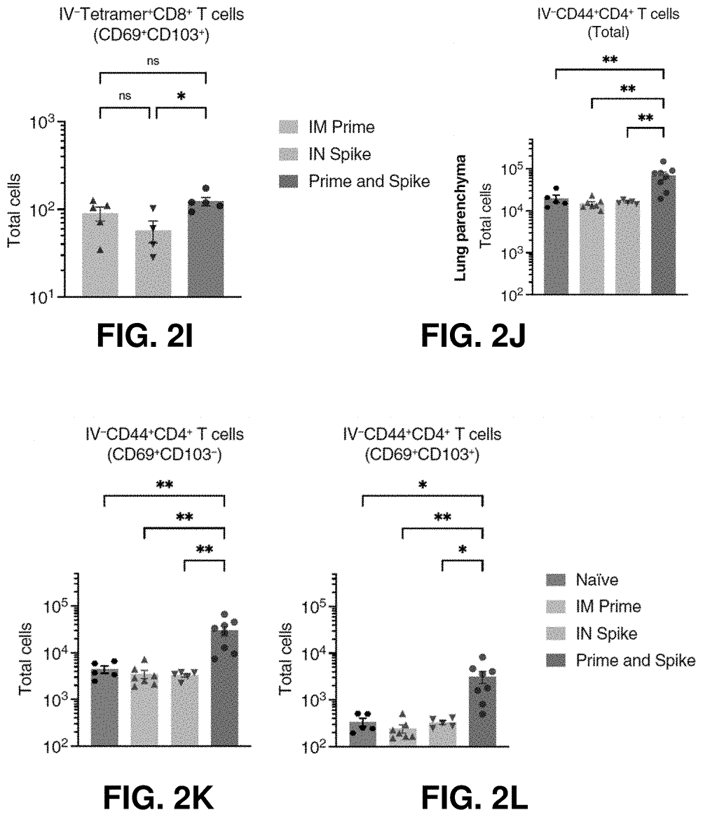

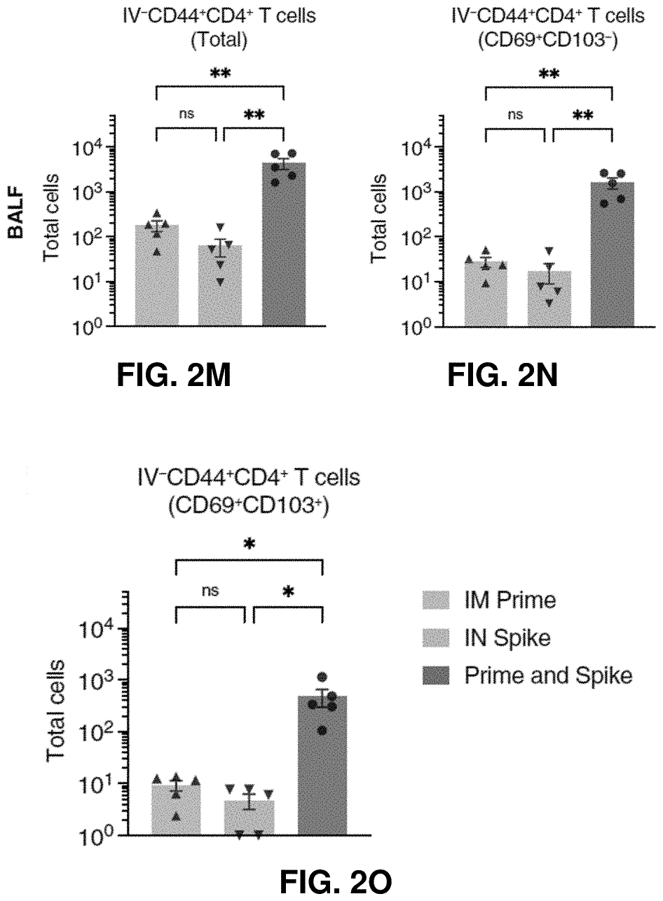

Given that P&S induced respiratory mucosal humoral memory, next studies assessed the induction of lung memory T cells (T_{RM} cells). Although adjuvant-free subunit vaccines have not traditionally been potent inducers of antigen-specific T cell responses, the hypothesis was that the immune memory generated by mRNA-LNP priming would enable subunit-mediated T cell-boosting responses. To identify spike-specific CD8⁺ T cells, major histocompatibility complex (MHC) class I tetramer S539-546 (VNFNFNGL (SEQ ID NO:6)) was used. There was a significant induction of IV-CD45⁻tetramer⁺ CD8⁺ T cells, which expressed canonical markers of T_{RM} cells including CD69⁺ and CD103⁺,within lung tissue (FIGS. 2A to 2C), BALF (FIG. 2D to 2F), and the nasal turbinate (FIG. 2G to 2I). Moreover, there were significant increases in antigen-experienced CD4⁺ T cells (IV⁻CD45⁻CD44⁺CD4⁺), many of which also expressed CD69⁺ and CD103⁺ both within lung tissue (FIG. 2J to 2L) and in the BALF (FIG. 2M to 2O). Thus, P&S mediates expansion of lung parenchyma and airway CD8⁺ T_{RM} and CD4⁺ T_{RM} cells.

Host Genotype, Boosting Interval, and IN Volume have Little Effect on P&S

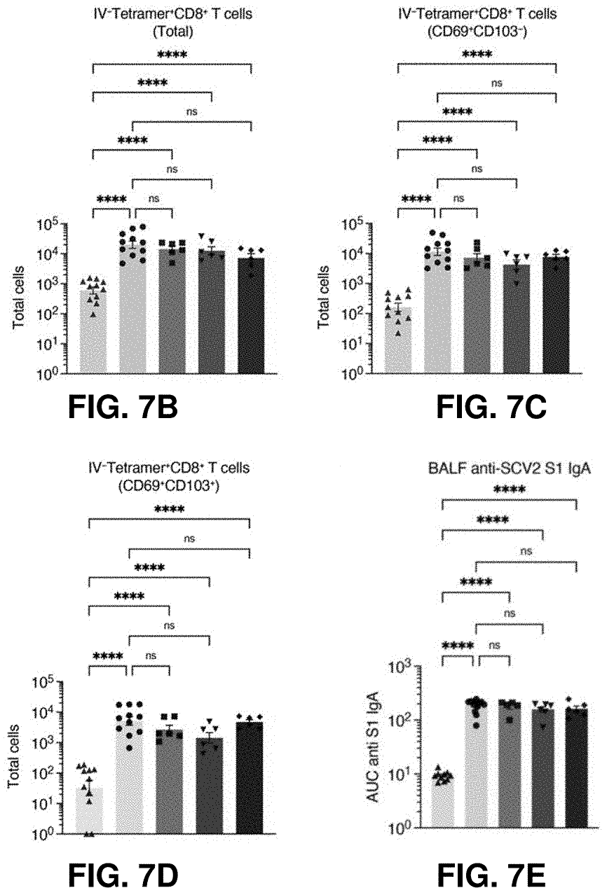

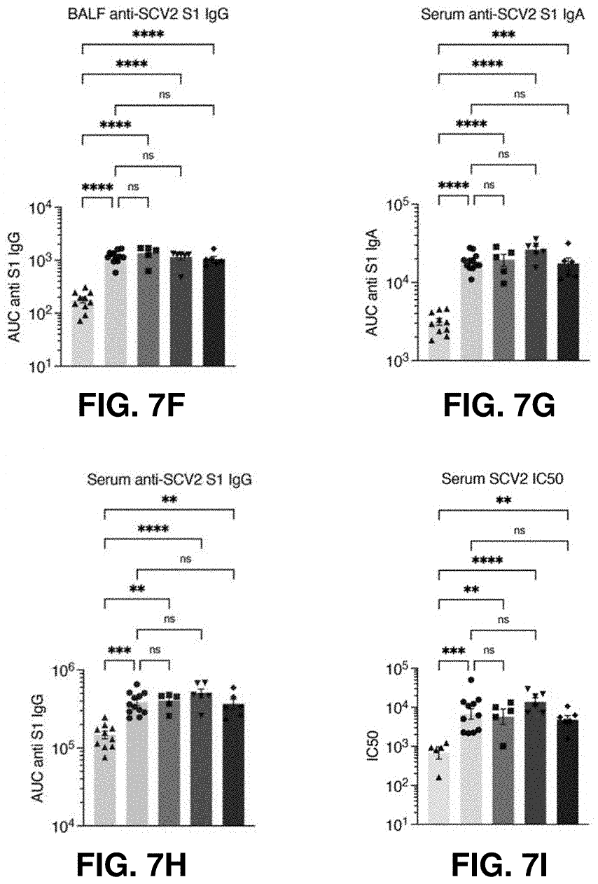

To assess whether mouse genotype, boosting interval, or boosting volume affected immunity induced by P&S, we compared mucosal CD8⁺ T cell and antibody responses after P&S under varying conditions, including in K18-hACE2 versus C57B6/J (B6J) mice, 2-week versus 4-week boosting intervals, and 25- versus 50 μl IN inoculations (FIG. 7A). Antigen-specific lung CD8⁺ T_{RM} cells (FIG. 7, B to D), BALF IgA and IgG (FIGS. 7E and 7F), serum IgA and IgG (FIGS. 7G and 7H), and serum neutralizing responses (FIG. 7I) were similar among all groups and significantly higher than responses elicited by prime alone. These results support the robustness of P&S because multiple experimental variables can be modified without affecting over-all immune responses.

Delayed-Interval P&S Induces Mucosal Immunity

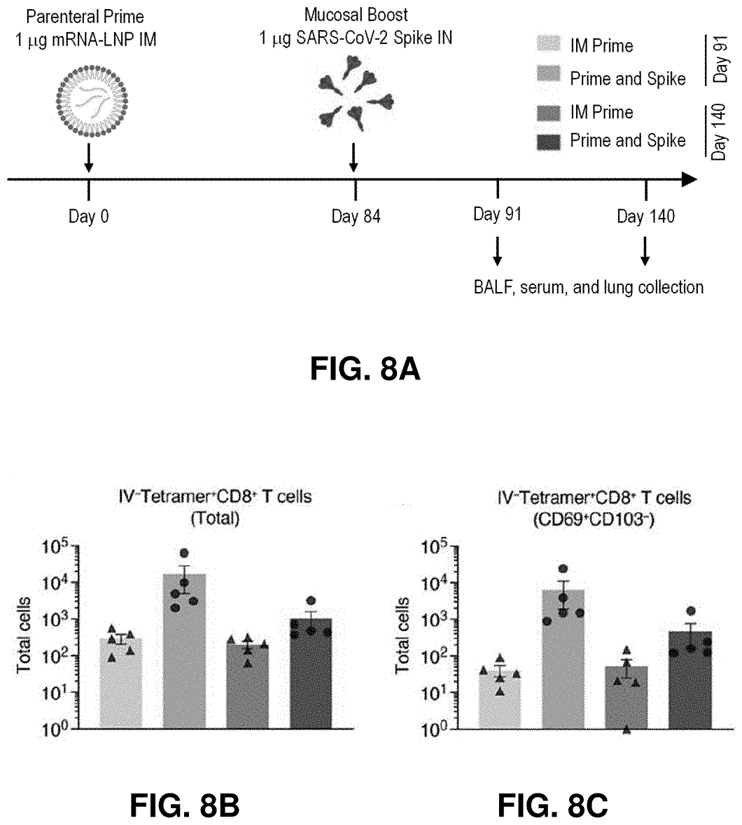

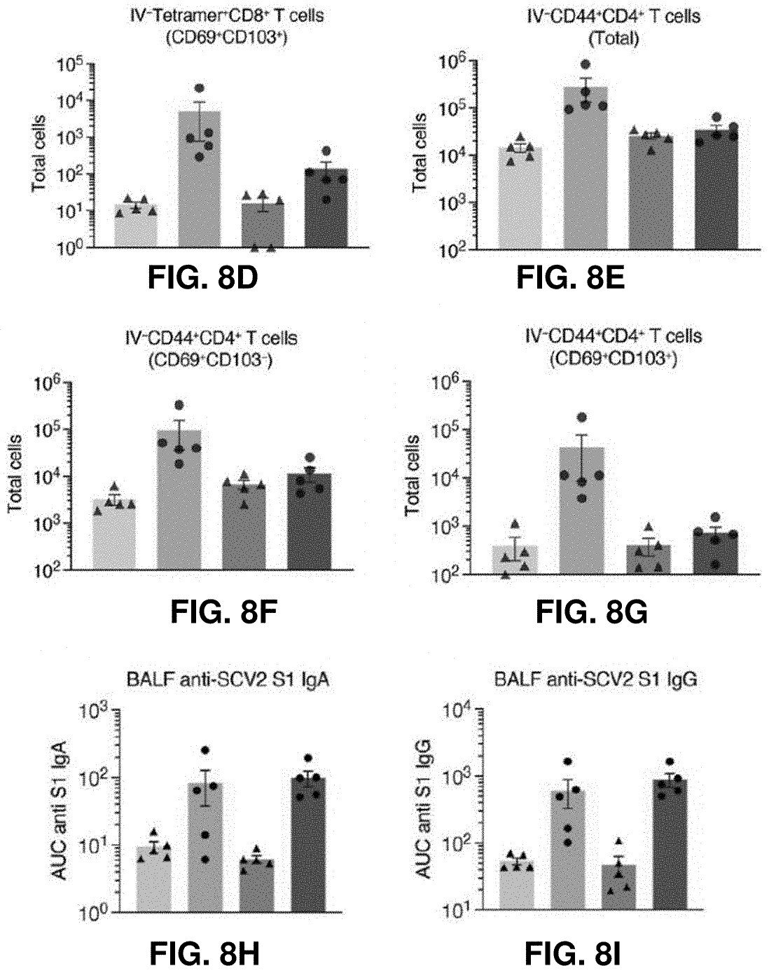

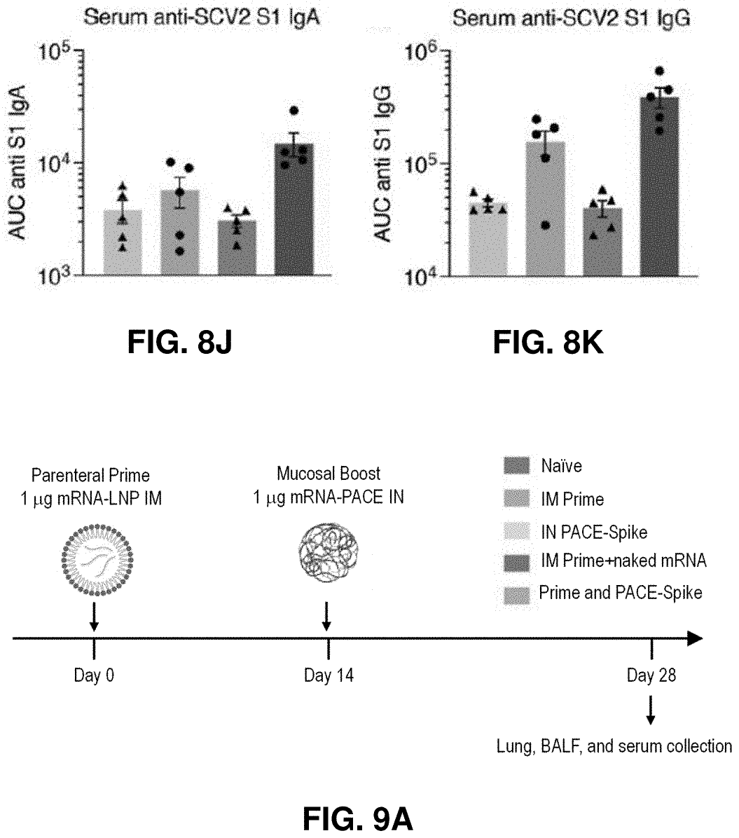

Additional studies tested whether boosting at an increased interval would affect P&S responses. To test this, mice received IM mRNA-LNP and were boosted with IN spike 84 days later. Hu-moral and cellular mucosal immune responses on days 91 and 140 were sampled (FIG. 8A). Delayed P&S was sufficient to induce CD8⁺ T_{RM} cells for at least 56 days (FIG. 8B to D). Polyclonal CD4⁺ T_{RM} cells were induced early at 7 days after boost. However, their numbers appeared to wane by 56 days (FIGS. 8E to G). Delayed P&S also resulted in enhanced mucosal IgA and IgG in BALF (FIGS. 8H and I) and serum IgA and IgG (FIGS. 8J and K) at 56 days after boost. Thus, P&S administered even up to 3 months after priming elicits durable mucosal humoral and cellular immune responses.

IN Delivery of mRNA Polyplexes Also Mediates Mucosal Boosting

Poly(amine-co-ester)s (PACEs) are biodegradable terpolymers that have been developed to encapsulate and deliver nucleic acids such as mRNA to specified tissues in vivo (Gun, et al., Biomaterials, 272:120780 (2021)). Recent studies have shown that mRNA-LNP delivered to the respiratory tract is lethal in a dose-dependent manner in mice (Ndeupen, et al., iSciene, 24:103479 (2021)). By contrast, PACE materials have been developed to be relatively immunologically silent, enabling administration to locations more susceptible to immunopathology such as the respiratory tract. Chemically modifying PACE with poly-ethylene glycol dramatically improves in vivo lung delivery. To assess the utility of PACE encapsulating mRNA encoding spike protein as an IN booster, mRNA was extracted from BNT162b2 and encapsulated in PACE. Mice were primed intramuscularly with mRNA-LNP and boosted with IN spike mRNA encapsulated in PACE (PACE-spike). Additional control groups included PACE-spike only and IM prime+extracted mRNA (naked mRNA) (FIG. 9A). Similar to what we found with P&S, prime and PACE-spike induced antigen-specific CD8⁺ T_{RM} cells (IV-CD45⁻tetramer⁺CD69⁺CD103⁺)(FIG. 9B to 9D). Additionally, PACE-spike-boosted mice developed high levels of BALF IgA. Levels of BALF IgG and serum IgA and IgG were similar to IM prime alone (FIG. 9E to 9H). IM prime followed by IN naked mRNA was unable to induce mucosal or systemic immune responses above that of IM prime alone, indicating that mRNA encapsulation by PACE was required for mucosal boosting. Additionally, a single dose of INPACE-spike alone was insufficient to elicit any detectable mucosal or systemic anti-body response at this dose.

IN Spike or IN PACE-Spike Boosts Suboptimal Prime to Protect Against Lethal SARS-CoV-2 Challenge

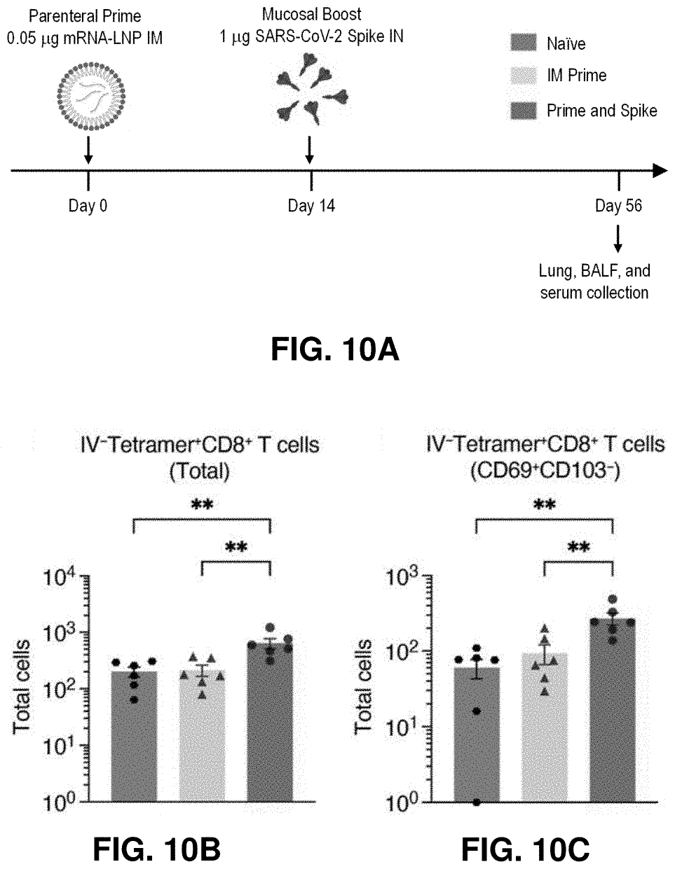

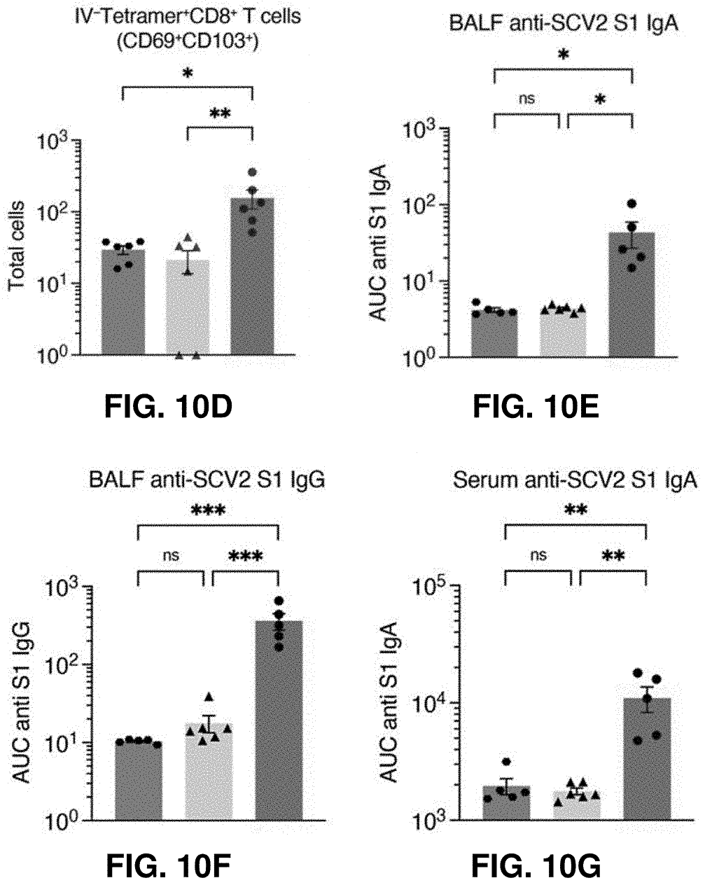

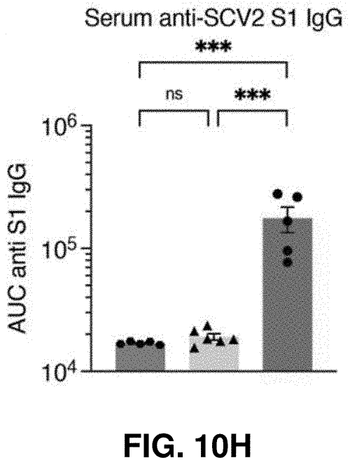

Although current vaccines were initially extremely effective at eliciting protective im-munity, waning antibody levels and immune evasion will necessitate boosters for the fore-seeable future. The best approach to boosting remains an open question. To test whether IN administration would provide an alternative protective boost, we used a low-dose (LD) 0.05 mg of mRNA-LNP vaccine to mimic nonprotective immunity. We have previously shown that this dose is insufficient to protect from SARS-CoV-2 challenge despite inducing systemic antibody responses (Israelow, et al., Immunol., 6, eabl4509 (2021). Mice intramuscularly primed with LD mRNA-LNP and boosted with IN spike developed antigen-specific lung CD8⁺ T_{RM} cells and IgA and IgG in the BALF at 42 days after boost (FIG. 10A-10H). Thus, low levels of immune memory allow for effective mucosal boosting of humoral and cellular responses by unadjuvanted IN spike.

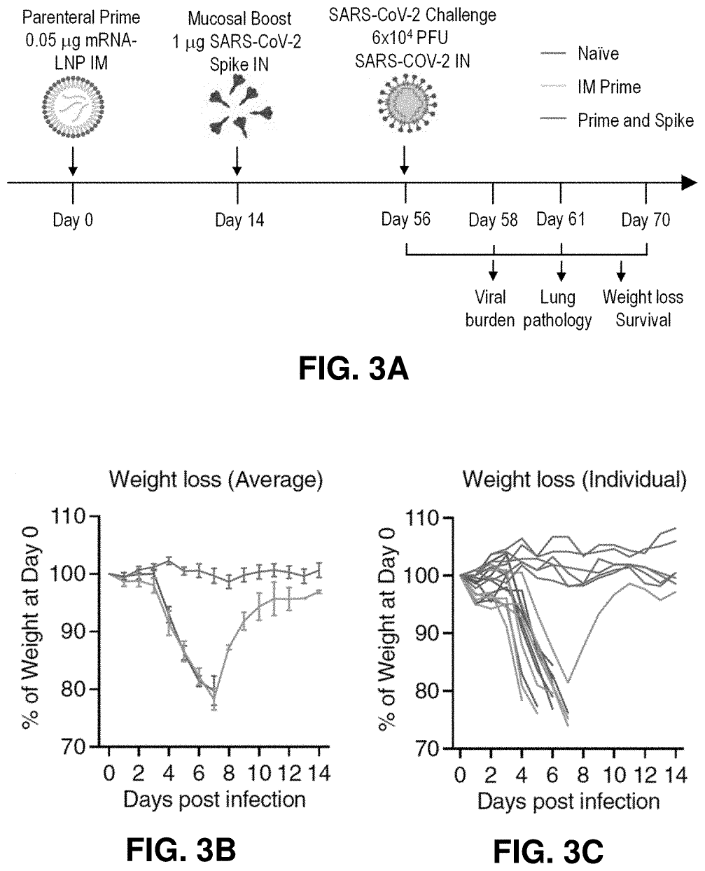

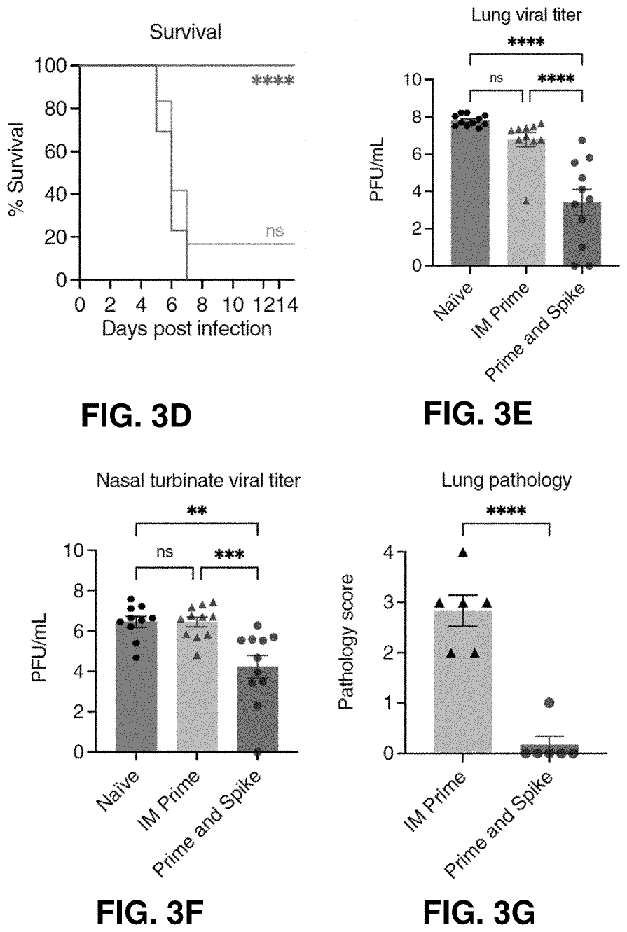

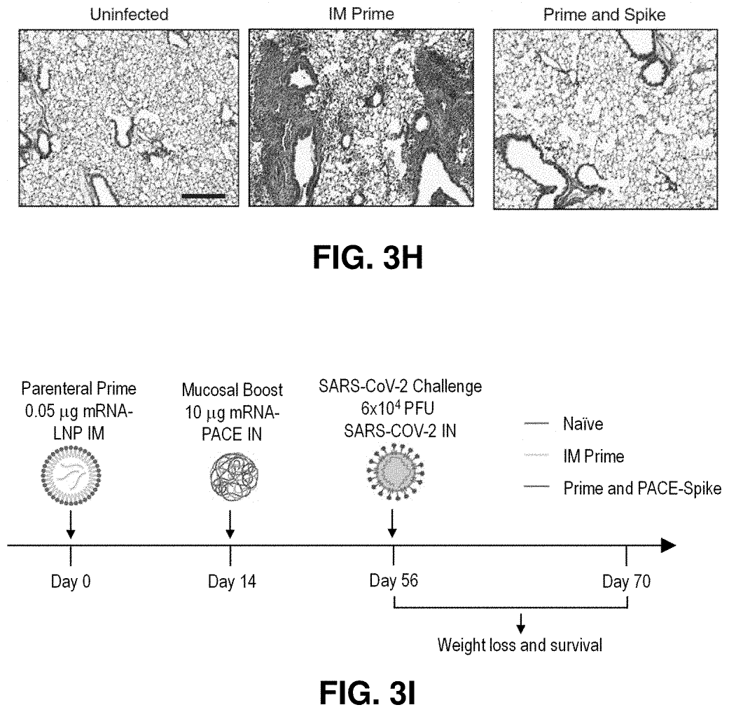

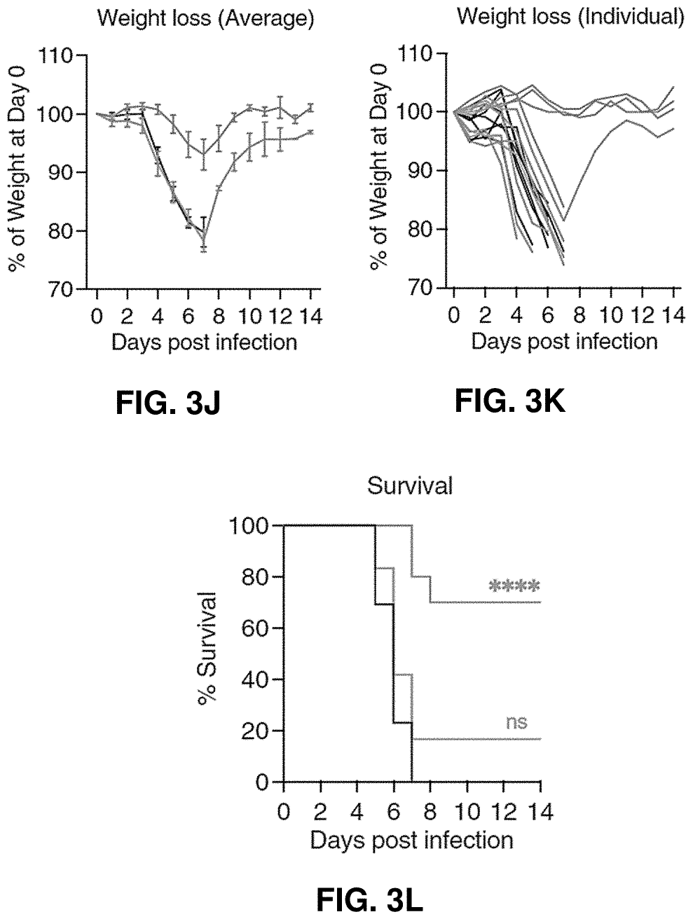

Naïve, LD prime only, or LD P&S mice were challenged with SARS-CoV-2 and assessed for viral burden at 2 days after infection, assessed for lungs pathology at 5 days after infection, or monitored for weight loss and mortality for 14 days (FIG. 3A). All mice vaccinated with P&S were completely protected from weight loss or death, but neither naïve nor LD prime-only mice were protected (FIGS. 3B to 3D). This protection was accompanied by reduced viral burden in both the upper respiratory tract (nasal turbinates) and lower respiratory tract (lungs) (FIGS. 3E and 3F). Furthermore, P&S led to significant protection from lung pathology, with only one of six mice developing limited mononuclear infiltrates at 5 days after infection (FIGS. 3G and 3H). Next, to assess the protective capacity of PACE-spike IN boost, we again immunized mice with LD mRNA-LNP intramuscularly and boosted them intranasally with PACE-spike mRNA. Prime and PACE-spike resulted in significant protection from morbidity and mortality (FIGS. 3I to 3L). Thus, both P&S and PACE-spike represents a robust, versatile, and safe vaccine strategy because IN boosting by either IN unadjuvanted spike or PACE-spike is sufficient to induce mucosal immunity and to provide protection against lethal challenge and COVID-19-like pulmonary disease.

P&S Achieves Robust Systemic Booster Responses Similar to Parenteral mRNA-LNP

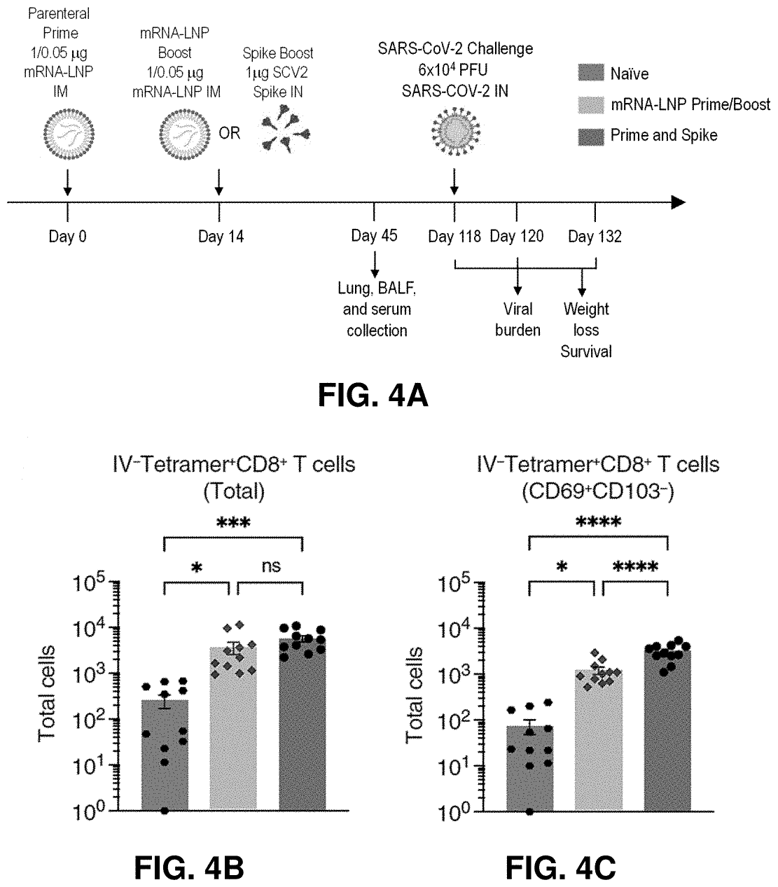

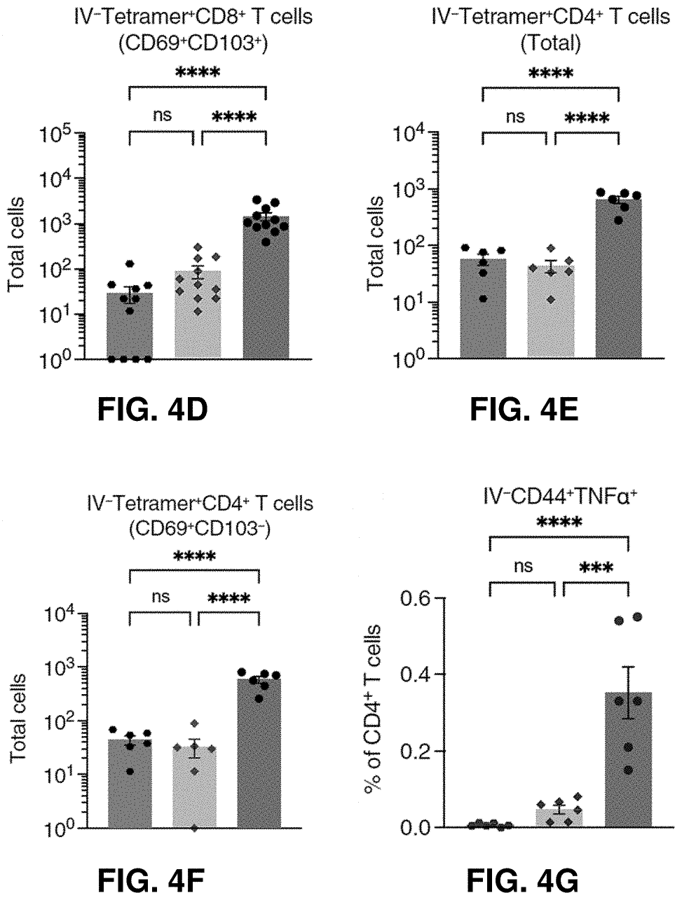

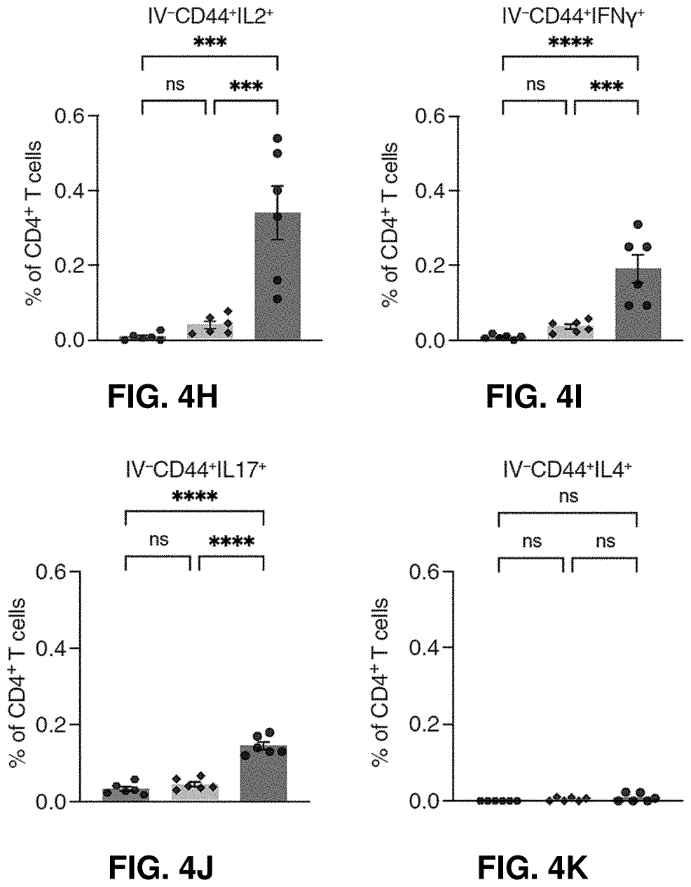

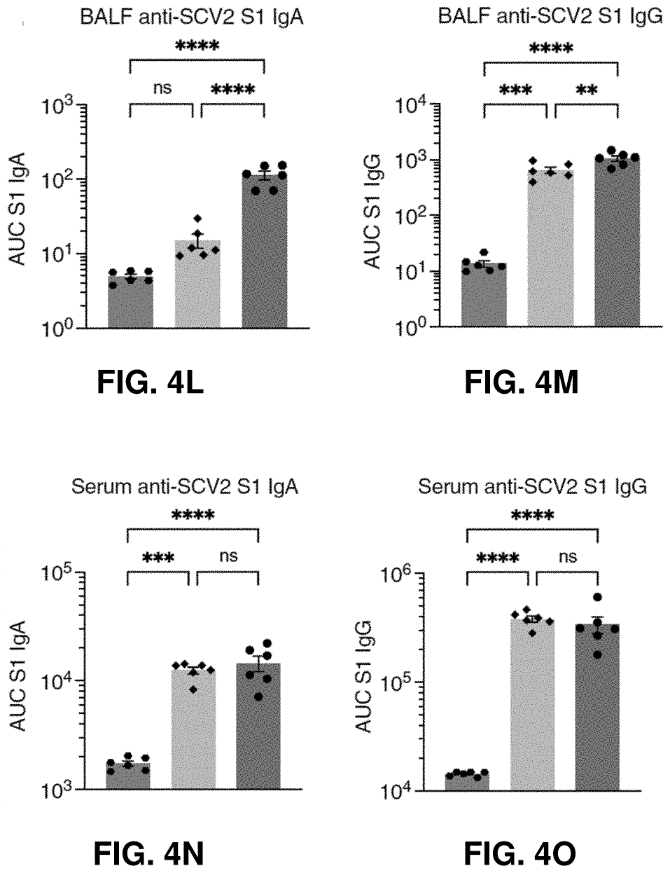

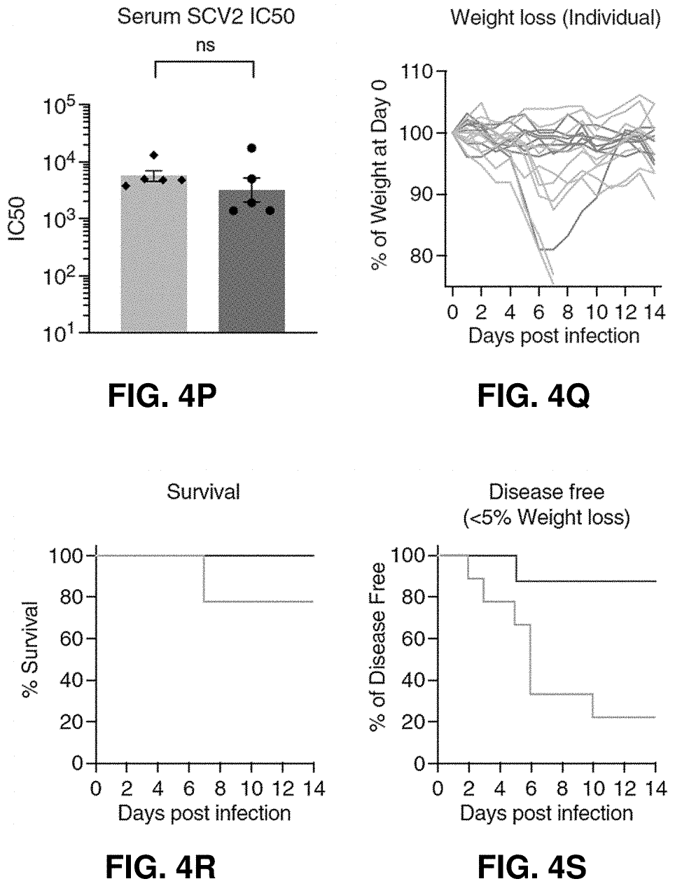

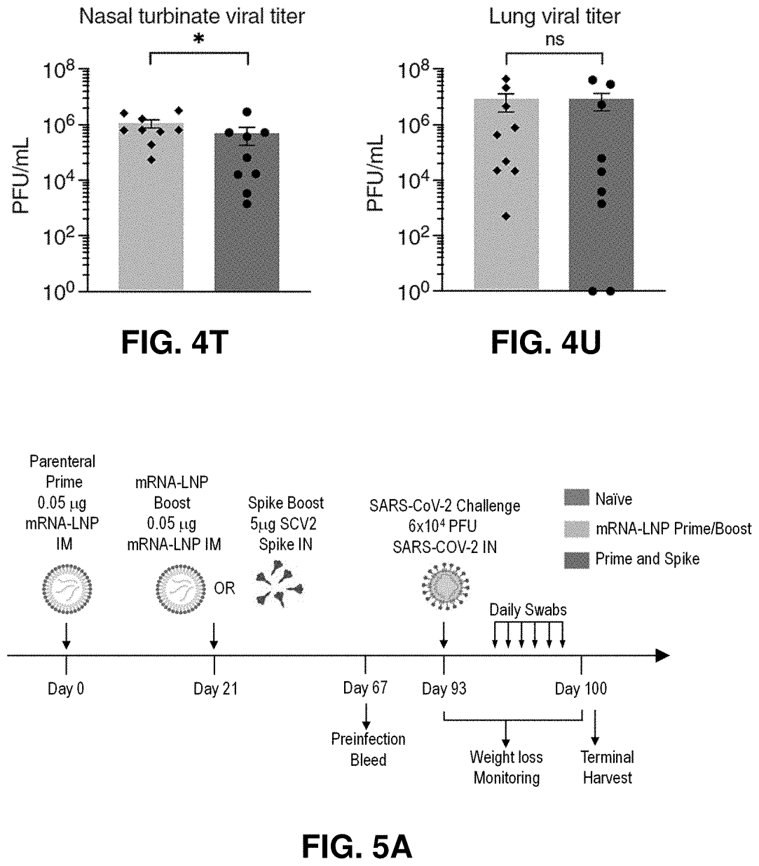

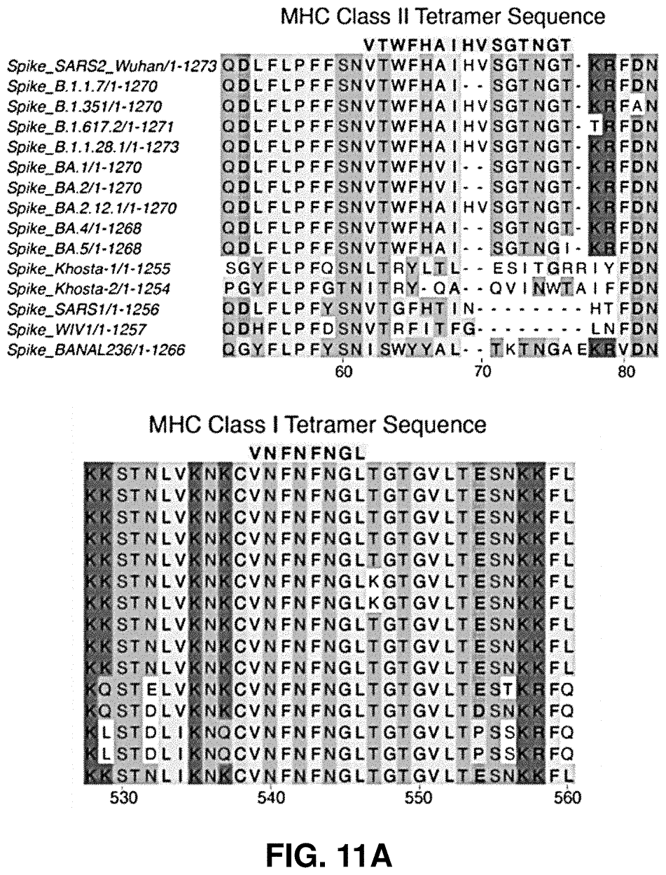

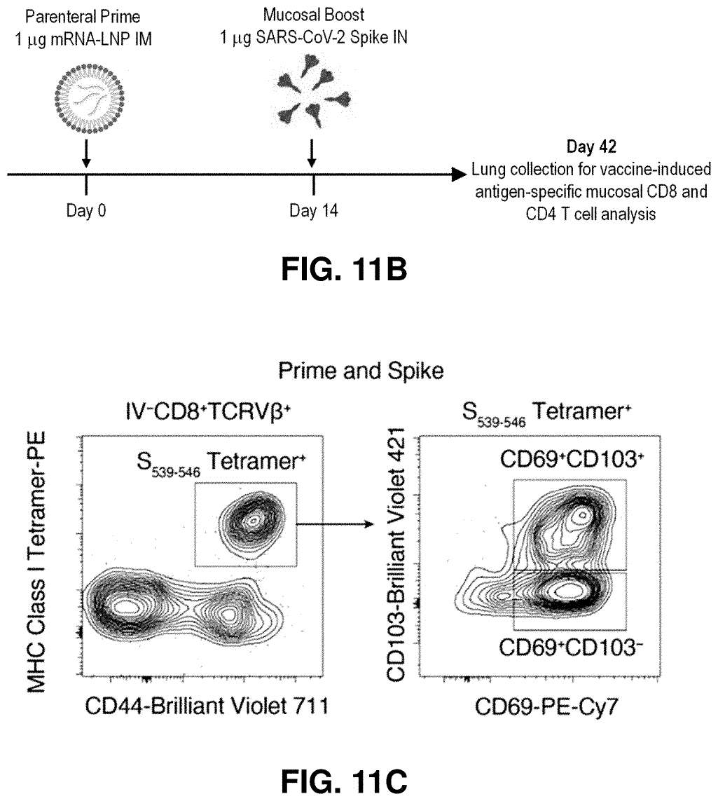

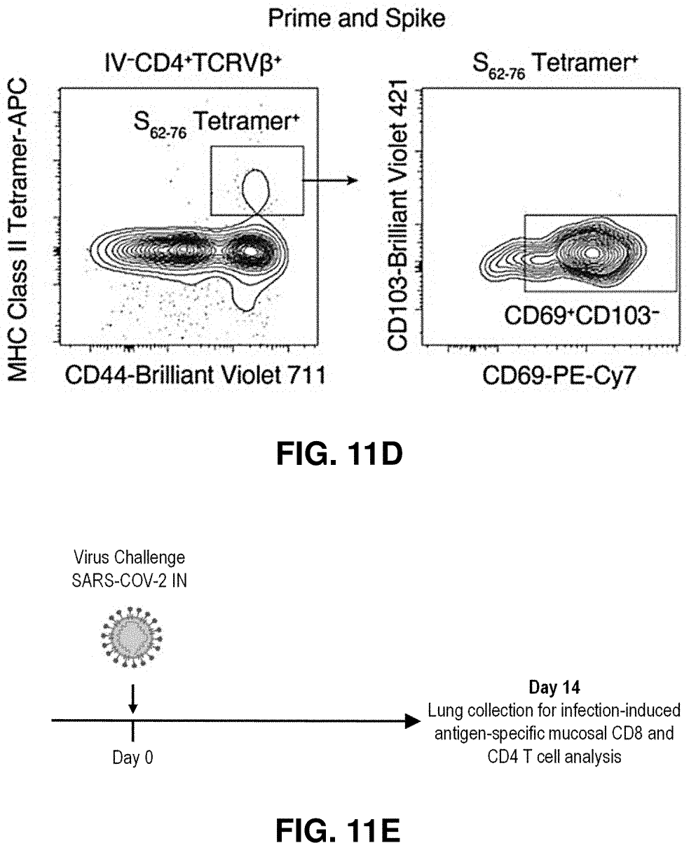

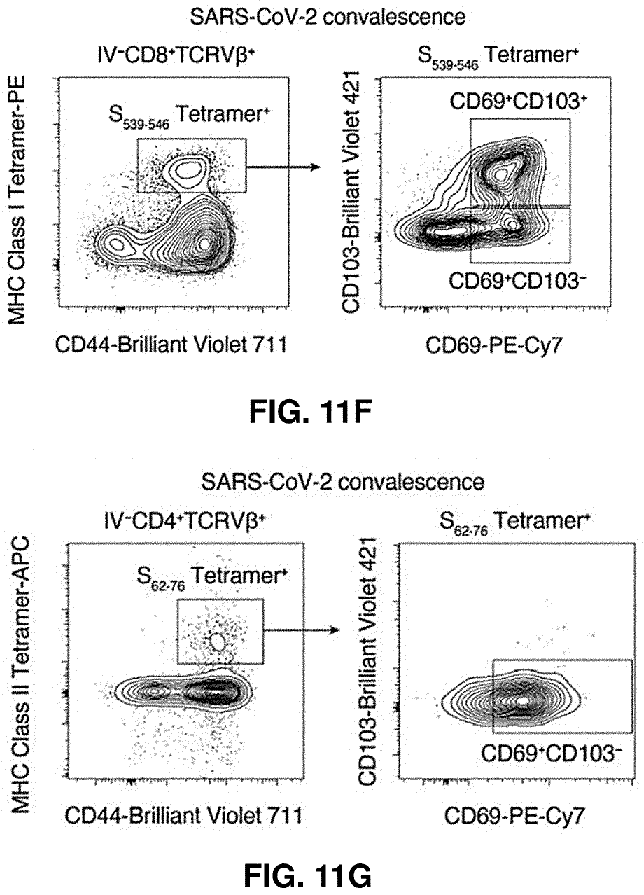

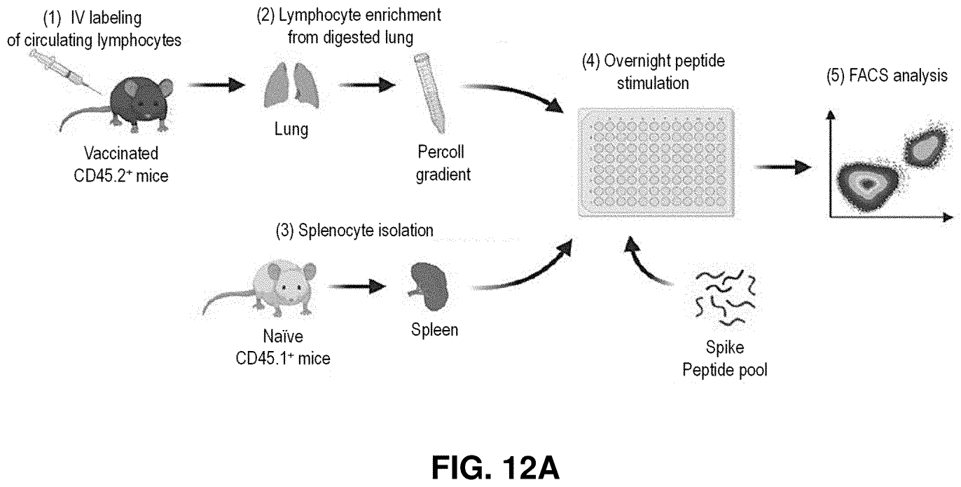

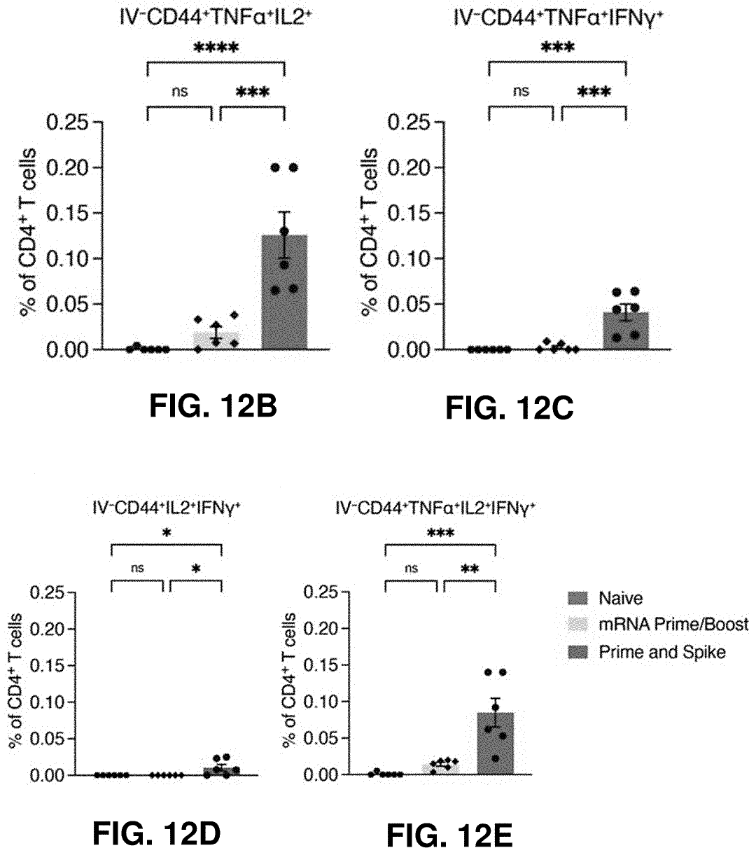

IM mRNA-LNP-based boosts are the current standard. Thus, we compared systemic and mucosal immune responses in P&S-vaccinated and IM mRNA-LNP prime-boost-vaccinated mice (FIG. 4A). Only P&S-vaccinated animals developed lung IV⁻CD45⁻tetramer⁺CD8⁺ T cells that express CD69+ and CD103⁺ (FIG. 4B to 4D). The peptide sequence corresponding to spike 62-76 (S62-76) is an epitope recognized by CD4⁺ T cells in convalescent C57BL/6 mice. A MHC class II tetramer S62-76 (VTWFHAIHVSGTNGT (SEQ ID N0:5)) was therefore developed, that readily identified lung-resident CD4⁺ T cells in both P&S and convalescent mice (FIG. 11A-G). Both infection and vaccination similarly led to increased IV⁻CD45⁻tetramer⁺CD4⁺CD69⁺CD103⁻ T_{RM} cells. P&S induced significantly higher levels of lung-resident antigen-specific CD4⁺ cells that phenotypically resemble infection-induced CD4⁺ T cells (IV-CD45⁻tetramer⁺CD69⁺CD4⁺) (FIGS. 4E and 4F). To further characterize the CD4⁺ T_{RM} cell response, we used a peptide stimulation assay and found that P&S led to a higher number of tissue-resident CD4⁺ T helper type 1 (TH1) and TH17 but not TH2CD4⁺ T cells (FIG. 4G to 4K). P&S also led to the induction of polyfunctional lung resident TH1 cells (FIG. 12B to 12E).

P&S-vaccinated but not prime-boost-vaccinated animals developed increased levels of BALF IgA (FIG. 4L). Although BALF IgG levels were increased in prime-boost relative to naïve, P&S developed significantly higher BALF IgG than that of prime-boost (FIG. 4M). Serum IgA and IgG in prime-boost- and P&S-vaccinated mice were similar (FIGS. 4N and 4O), as were neutralizing antibody levels (FIG. 4P). Thus, P&S induces similar systemic binding and neutralizing antibody levels a correlate of protection in humans and it uniquely elicits mucosal IgA, IgG, CD4⁺ T_{RM} cells, and CD8⁺ T_{RM} cells. Notably, only P&S elicits TH1 and TH17 CD4⁺ T_{RM} cells and not pathogenic TH2 cell responses, which have been associated with vaccine-associated enhanced disease (VAED) (Gartlan, et al., Fron. Immunol., 13:882972 (2022).

To compare the protective efficacy of P&S to mRNA-LNP prime-boost, mice were primed with LD (0.05 μg) mRNA-LNP and boosted with either LD mRNA-LNP (intramuscularly) or 1 μg unadjuvanted spike protein (intranasally). Mice were challenged 118 days after prime. Both vaccine strategies led to roughly equivalent protection from death, with two of nine prime-boost mice and zero of nine P&S mice succumbing to infection (FIGS. 4Q and 4R). P&S led to significantly enhanced disease-free survival indicated by only one of nine mice losing >5% of initial body weight, whereas six of nine mRNA-LNP prime-boost mice lost >5% of their starting body weight (FIG. 4S). P&S also led to enhanced upper-airway protection, indicated by decreased nasal turbinate viral load, and reduced although not statistically significant lower airway viral load (FIGS. 4T and 4U).

P&S Reduces Transmission in a Hamster Model of SARS-CoV-2

Next, we used Syrian hamsters to assess both the viability of P&S in an alternate SARS-CoV-2 model and its ability to reduce transmission. Hamsters were vaccinated by means of either IM mRNA-LNP prime-boost or P&S (FIG. 5A). Serum IgA and IgG levels at 67 days after prime were equivalent between the two groups (FIGS. 5B and 5C). Hamsters were infected with SARS-CoV-2 at 93 days after prime, and both groups were equivalently protected from disease, as indicated by minimal weight loss and reduced lung pathology relative to those of naïve animals (FIGS. 5D and 5E). P&S-vaccinated animals cleared viral shedding more quickly relative to naïve controls starting at 4 days after infection, with all oral swabs negative for infectious virus by 5 days after infection. Conversely, mRNA-LNP prime-boost animals did not have significantly lower titers at 4 or 5 days after infection and did not stop shedding virus until 6 days after infection. Cumulative viral shedding assessed with area under the curve (AUC) revealed that both mRNA-LNP prime-boost and P&S-vaccinated animals had significantly lower over-all viral shedding than naïve animals. Although P&S AUC was less than mRNA-LNP prime-boost, the results were not statistically significant.

Although P&S reduced viral shedding after infection, the question of whether P&S was able to reduce transmission to vaccinated animals was not yet answered. Vaccinated hamsters were therefore cohoused with naïve donor hamsters that had been infected 24 hours prior (FIG. 5J). P&S-vaccinated contact hamsters had significantly lower viral titers at days 2, 4, and 5 after exposure relative to naïve, whereas mRNA prime-boost-vaccinated animals did not have significantly reduced viral shedding at any single time point after exposure (FIGS. 5L to 5N). Both P&S and mRNA prime-boost were equally protected from lower respiratory tract pathology in the setting of transmission (FIG. 5K and data not shown). Peak viral load (at 2 days after infection) and cumulative viral shedding were significantly reduced in P&S-vaccinated animals relative to both naïve and mRNA-LNP prime-boost con-tact hamsters (FIG. 5O). Thus, P&S appears to be an effective vaccine strategy in hamsters and reduces viral transmission.

Heterologous Spike Robustly Elicits Cross-Reactive Immunity

Boosting at a distinct anatomic location, in this case, the respiratory mucosa by homologous unadjuvanted subunit spike enables the formation of new mucosal immune memory and enhances systemic immunity. However, VOCs such as current Omicron sublineages have substantial changes to the spike protein sequence, leading to evasion of preexisting humoral immunity. It is likely that future variants will diverge even more, which suggests that a boosting strategy that elicits broadly reactive immunity will be necessary to neutralize future variants.

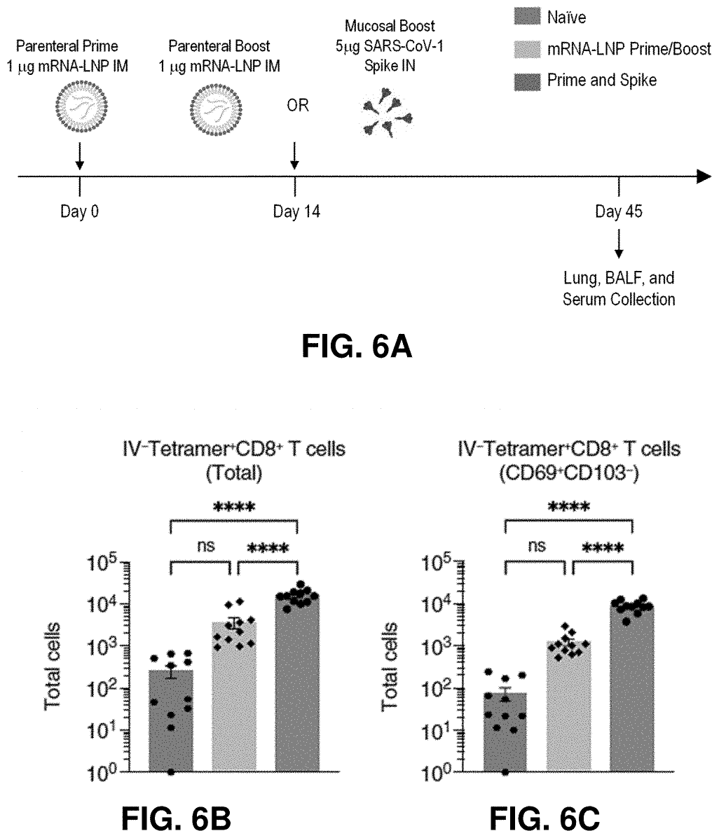

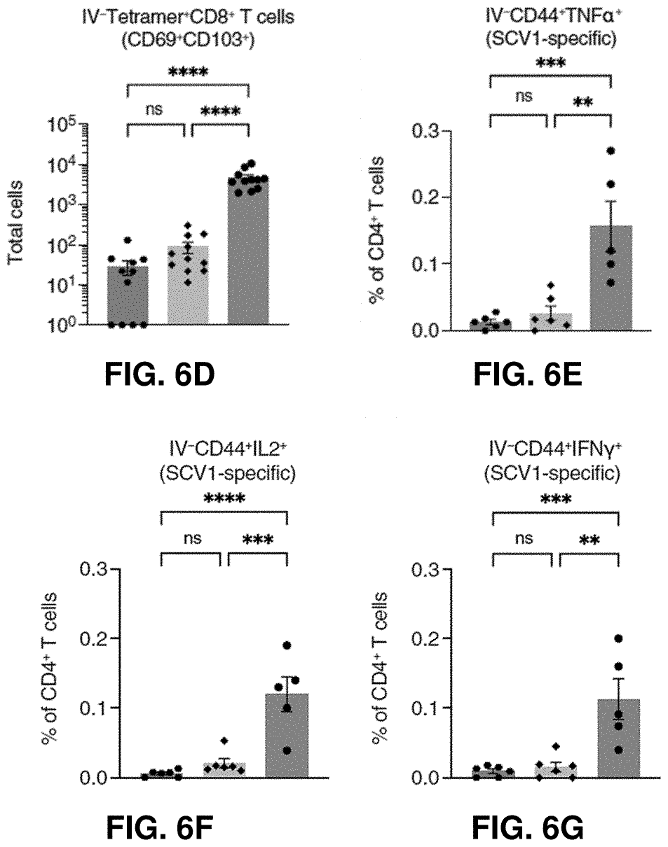

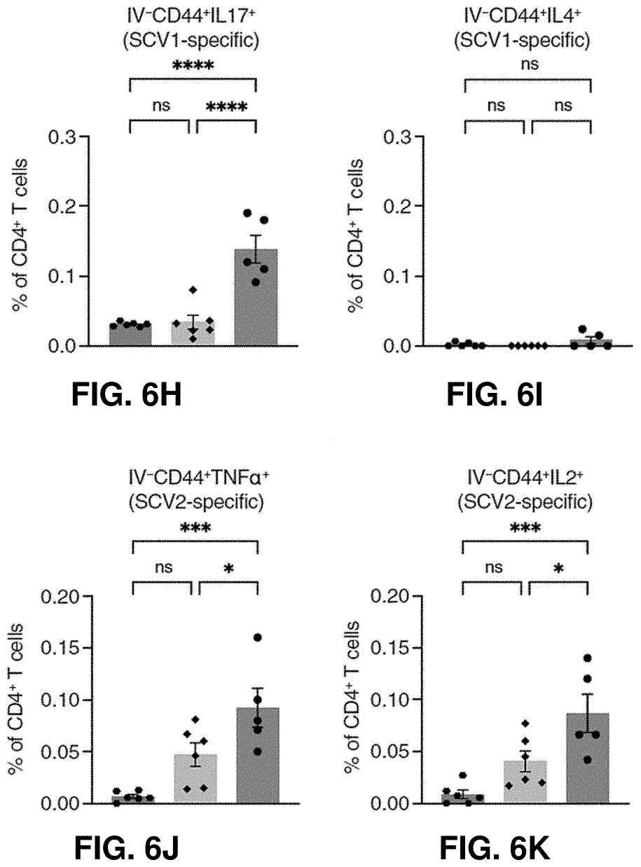

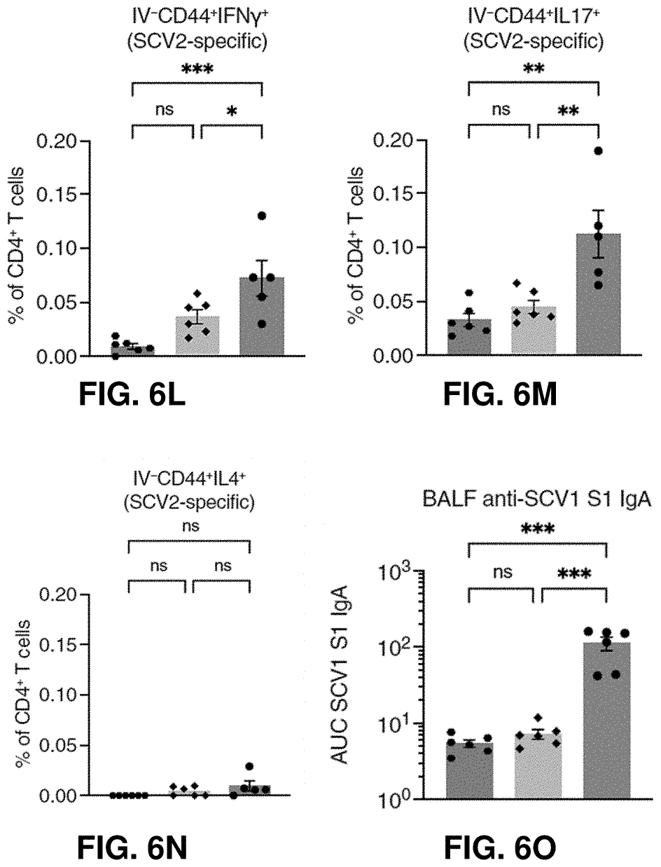

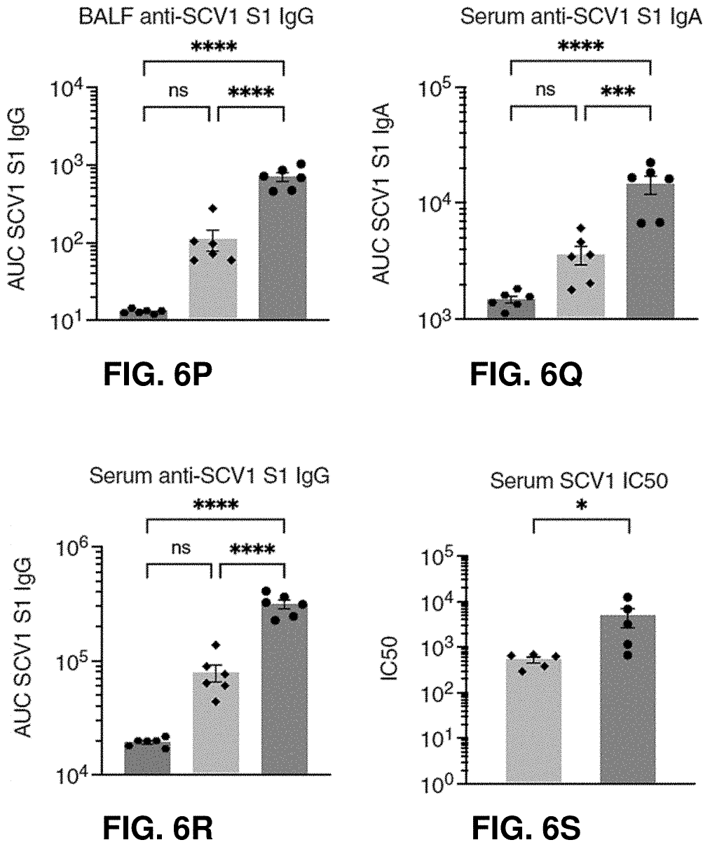

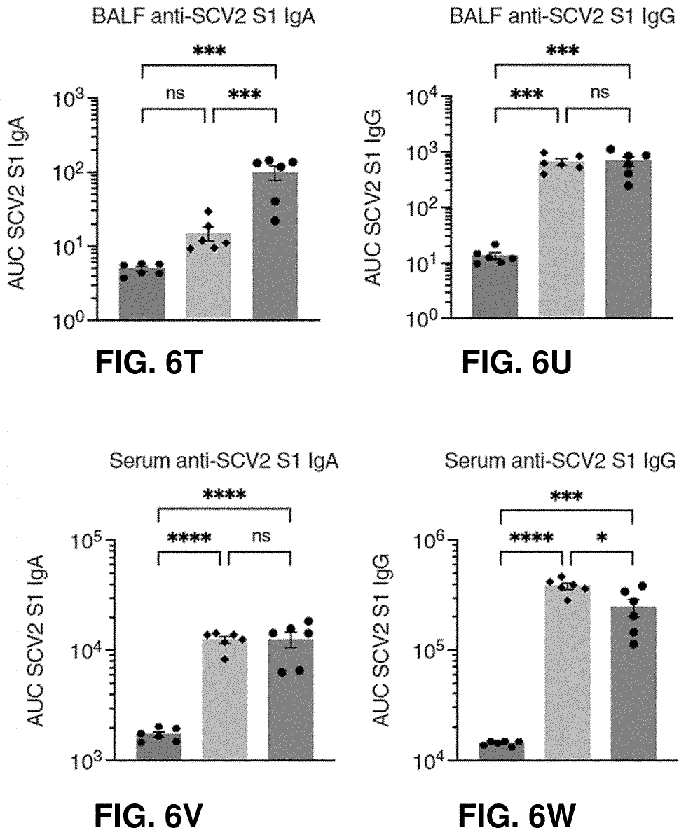

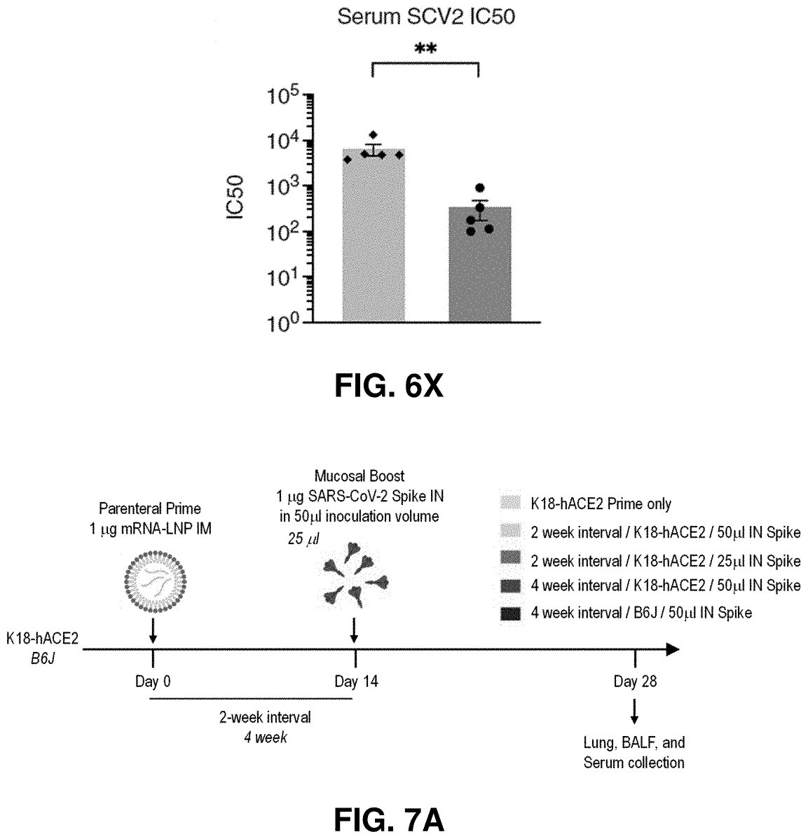

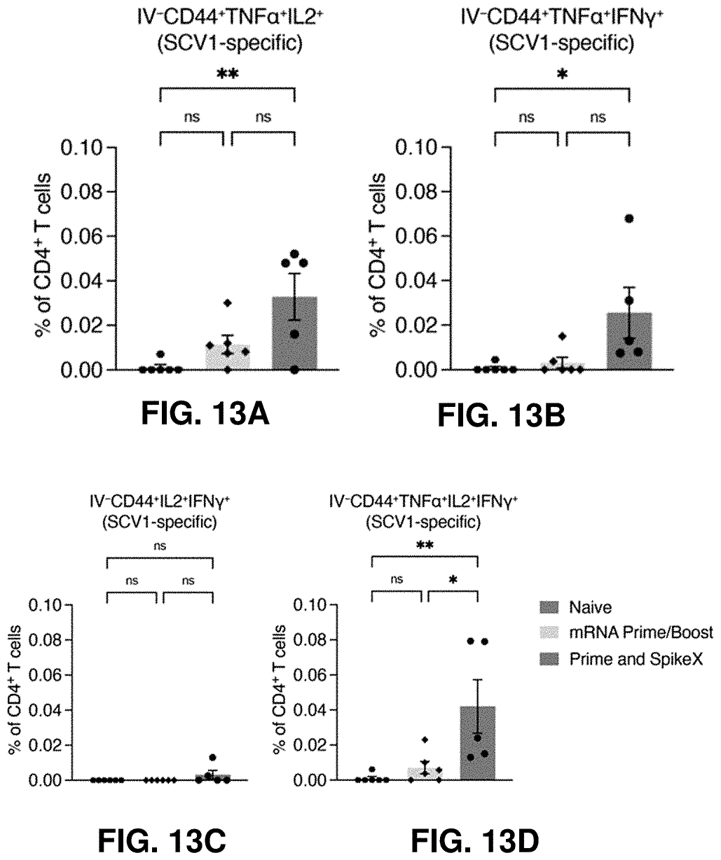

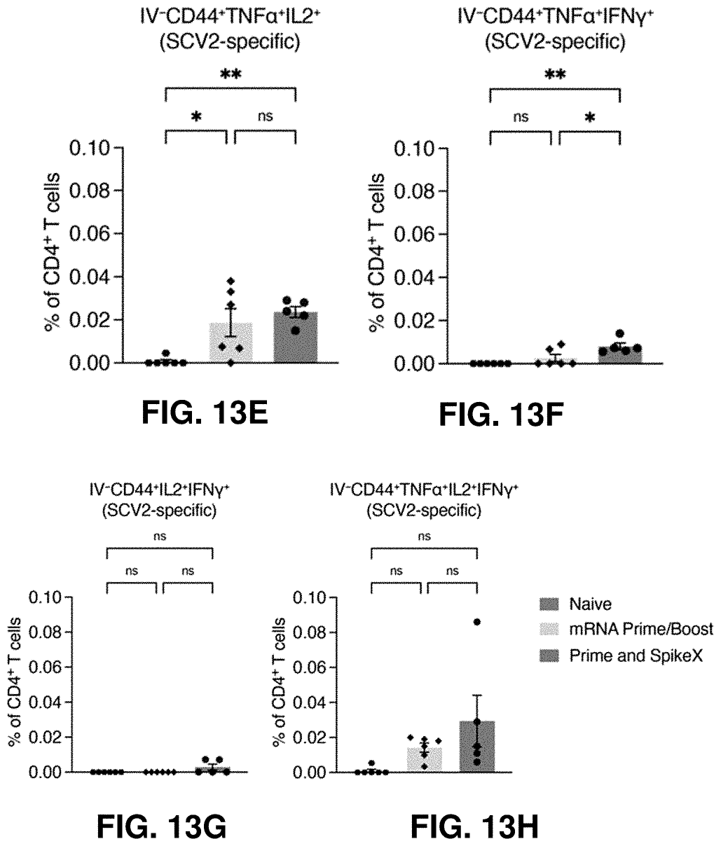

To test the ability of an unadjuvanted heterologous spike (Spike X) protein in P&S, K18-hACE2 mice were primed with SARS-CoV-2 mRNA-LNP followed by IN boosting with SARS-CoV-1 spike, which we refer to as P&Sx (FIG. 6A). Although SARS-CoV-1 is a related sarbecovirus, its spike protein only shares 76% homology with SARS-CoV-2 spike. At 45 days after prime, there were increased IV⁻CD45⁻ tetramer⁺ CD8⁺ T_{RM} cells (FIG. 6B to 6D). The MHC class I tetramer sequence was highly conserved within the sarbecovirus family (FIG. 11A). A peptide stimulation assay was performed using both SARS-CoV-1 and SARS-CoV-2 peptide pools to assess the development of antigen-specific lung CD4⁺ T_{RM} cells. The data showed that P&Sx led to both the development of SARS-CoV-1-specific and to a lesser extent SARS-CoV-2-specific antigen-specific TH1 and TH17 CD4⁺ T_{RM} cells and no induction of CD4⁺ T_{RM} expressing the TH2 cytokine interleukin-4 (IL-4) (FIG. 6E to 6N, and FIG. 13A-13H). There were also increased anti-SARS-CoV-1 IgA and IgG in both the BALF and serum in P&Sx relative to IM mRNA-LNP prime-boost (FIG. 6O to 6R). P&Sx mice correspondingly developed higher neutralization titers against SARS-CoV-1 than those of mice vaccinated with SARS-CoV-2 mRNA-LNP prime-boost (FIG. 6S). P&Sx induced higher anti-SARS-CoV-2 BALF IgA than SARS-CoV-2 mRNA-LNP prime-boost and similar levels of anti-SARS-CoV-2 IgG in BALF (FIGS. 6T and 6U). Consistent with the elevated serum IgG levels, mRNA-LNP prime-boost mice had higher serum neutralization titers against SARS-CoV-2 than that of P&Sx mice (FIG. 6V to 6X). Thus, IN boosting with unadjuvanted heterologous spike protein can induce potent mucosal cellular and humoral memory against a substantially diver-gent sarbecovirus.

### Example 2. Systemic mRNA Prime Followed by Mucosal mRNA Boost as a Hybrid Strategy to Establish Tissue-Resident Immunity

Mucosal exposure to exposure to cognate antigens and/or inflammation is key to the development of tissue-resident memory T and B cell responses. It has been established that parenteral mRNA vaccination fails to establish tissue-resident T cell immunity that is elicited by natural infection. Therefore, a systemic mRNA prime, followed by mucosal mRNA boost was proposed as a hybrid strategy to establish tissue-resident immunity.

Methods and Materials

Poly(amine-co-ester) (PACE) particles were prepared as previously described. Briefly, building blocks were assembled into long-chain “PACE” molecules. PEG was attached to one end of one pool of the PACE, to form PACE-PEG molecules. Nucleic acid encoding polypeptide antigen was combined with different amounts of PACE and PACE-PEG molecules to form PACE particles (PACE nanoparticles).

The PACE used here has end group 14; Mw is 7.5+/−0.5 kDa with PDI of 2.2+/−0.1. The PACE-PEG Mw is 14+/−0.5 kDa with Mn of 12 kDa with PDI of 1.1+/−0.1. The PEG portion has a Mn of 5 kDa, and was purchased from Sigma Aldrich.

The size of the resulting nanoparticles on average is 160+/−25 nm at the concentration that is made for in vivo with PDI of 0.2+/−0.05 for 1-50% PP content. Particles made for in vitro experiments, made at much lower concentrations, have a smaller average particle size.

The prep was the usual method with a 1:50 ratio of mRNA to polymer, pH 5.8 sodium acetate buffer.

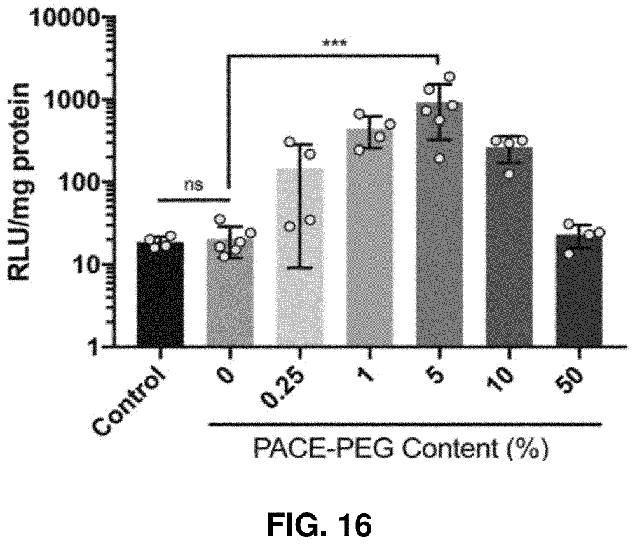

PEG-conjugated PACE-mediated delivery and expression of mRNA cargoes into the lung was demonstrated for each of control and samples containing 0%, 0.25%, 1%, 5%, 10%, or 50% PACE-PEG, respectively (FIG. 16).

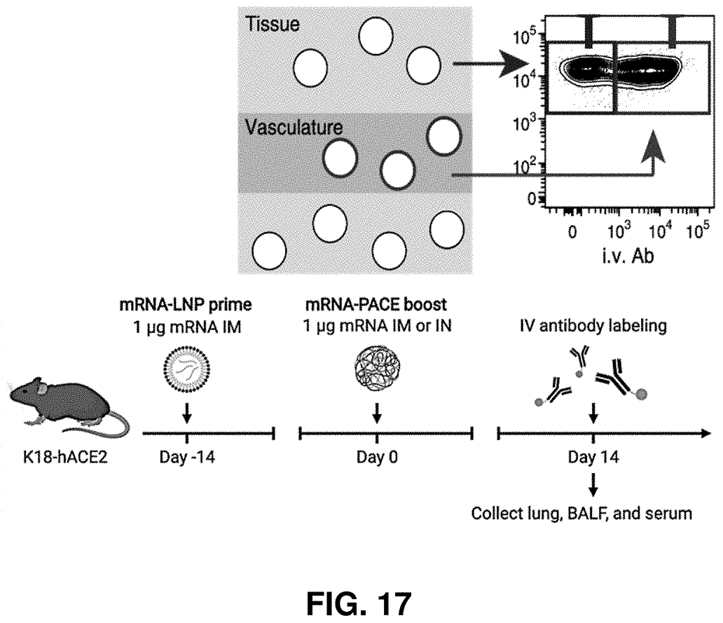

Methodologies for using mRNA-PACE as a mucosal boost agent to establish tissue-resident immunity against a respiratory virus were assessed using SARS-CoV-2 antigen. A schematic flow diagram for the methods is set forth in FIG. 17. Briefly, antigen (mRNA-lipid nanoparticles) at 1 microgram RNA were injected into a K18-hACE2 mouse model via the intramuscular (IM) route 14 days prior to boost (Day-14). A boost step (injecting mRNA-PACE particles at 1 microgram RNA, via intramuscular (IM) or intranasal (IN) route) was carried out at Day 0, and IV antibody labelling was conducted two weeks following the boost (Day 14), followed by collecting lung, bronchoalveolar lavage fluid (BALF) and serum.

Transgenic mice susceptible to SARS-CoV-2 infection (K18-hACE2) were (“primed”) injected with 1 ug in 10 ul of mRNA-LNP encoding SARS-CoV-2 spike protein or mock injected via intramuscular route. 14 days post later, mice were “boosted” with PACE-PEG particles containing 1 ug of mRNA encoding the SARS-CoV-2 spike protein via intranasal administration. For comparison, other primed mice received either intramuscular injection of PACE-PEG particles containing 1 ug of mRNA encoding the SARS-CoV-2 spike protein or mock injection. 14 days after boosting mice were euthanized. 3 minutes prior to euthanasia mice were injected intravenously with CD45 labeling antibody to differentiate vascular (IV+) from tissue resident (IV−) cells.

Lungs were digested at 37c for 45 minutes with RPMI supplemented with collagenase A and DNase, and subsequently homogenized into single-cell suspension. Lung single-cell suspensions were stained with fluorescently labeled antibodies and then fixed in 4% paraformaldehyde prior to acquisition on flow cytometer and analysis with FlowJo software. Serum and bronchioalveolar lavage fluid (BALF) were harvested and analyzed using enzyme linked immunoassay against (ELISA) SARS-CoV-2 spike S1 domain at the indicated dilutions. To perform ELISA, 96 well plates were coated with 50 microliters of 2 micrograms per ml of 51 protein in PBS overnight at 4° C. Plates were incubated for 1 hour at room temperature with 250 μl of blocking solution (PBS with 0.1% Tween 20 and 5% milk powder). Serum was diluted in dilution solution (PBS with 0.1% Tween 20 and 2% milk powder), and 100 μl of diluted serum was added for 2 hours at room temperature. Plates were washed five times with PBS-T (PBS with 0.1% Tween 20) and 50 μl of horseradish peroxidase anti-mouse IgG or anti-mouse IgA was added to each well. After 1 hour of incubation at room temperature, plates were washed three times with PBS-T. Plates were developed with 100 μl of trimethylboron substrate reagent set and the reaction was stopped after 15 min by the addition of 2 N of sulfuric acid. Plates were then read at a wavelength of 450 and 570 nm, and the difference reported.

Results

Intramuscular (IM) injection of mRNA-lipid nanoparticles (LNP), when used as a “prime” vaccination, followed by intra-nasal (IN) administration of mRNA-PACE, used as a “boost” vaccination 14 days post-priming, induced lung-resident (IV⁻) spike-specific CD8 T cell immunity, but did not affect circulatory (IV⁺) CD8 T cell immunity.

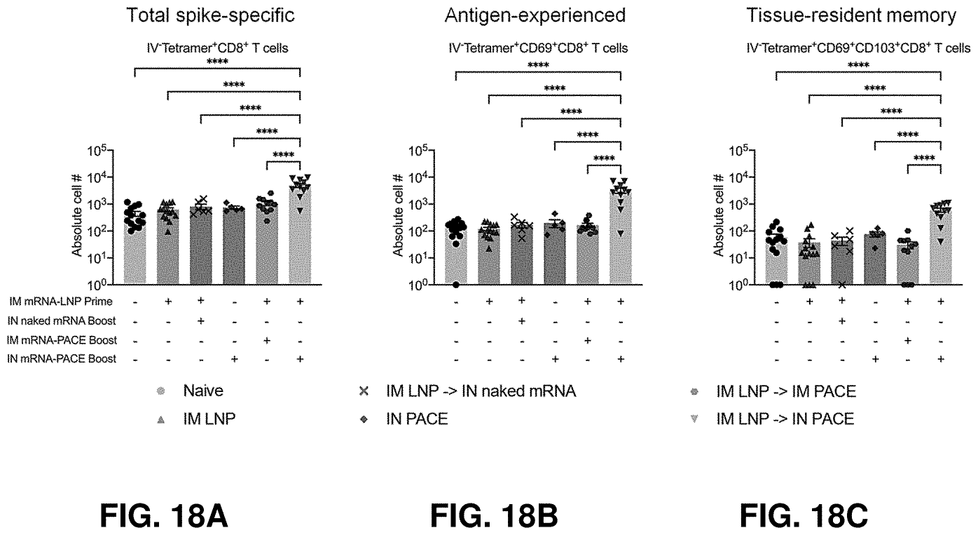

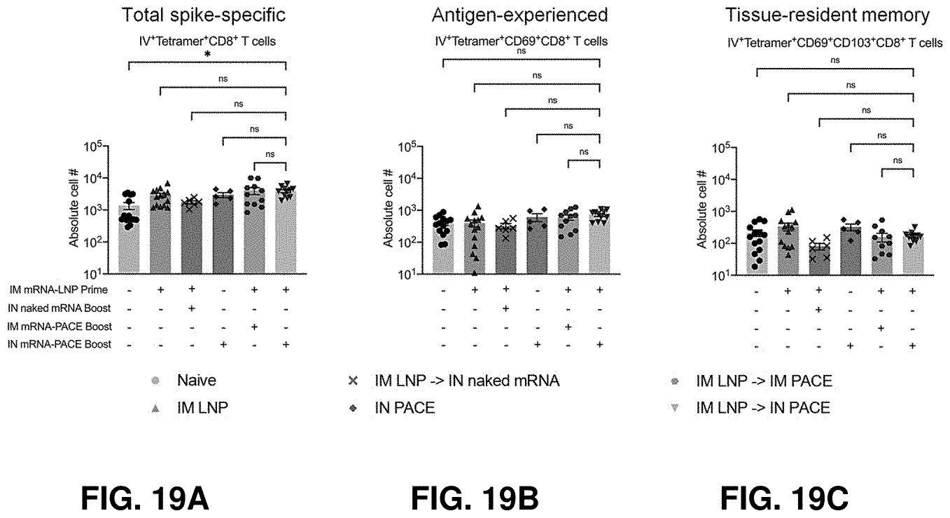

Results showing absolute cell numbers for each of samples (Naïve; IM LNP; IM LNP>IN naked mRNA; IN PACE; IM LNP>IM PACE; and IM LNP>IN PACE spike-specific CD8 T cells) are presented in FIGS. 18A-18C for “IV⁻” lung-resident T cells, and in FIGS. 19A-19C for IV⁺ circulating T cells, respectively.

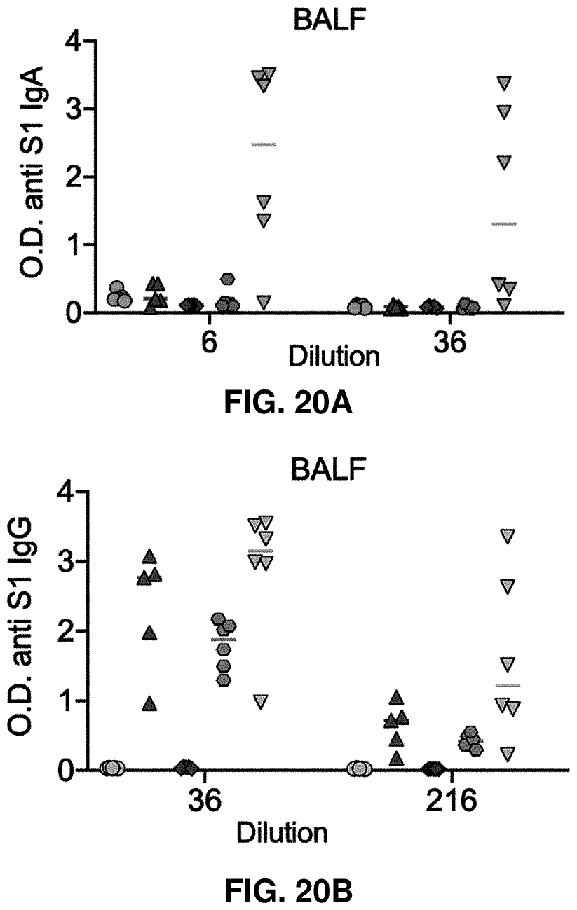

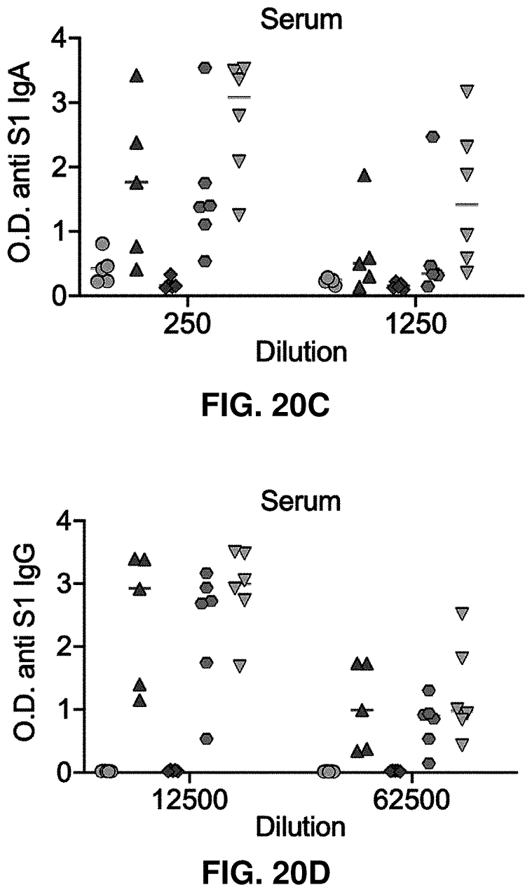

In addition, it was demonstrated that Intramuscular (IM) injection of mRNA-lipid nanoparticles (LNP), when used as a “prime” vaccination, followed by intra-nasal (IN) administration of mRNA-PACE, used as a “boost” vaccination 14 days post-priming, elicited robust mucosal and systemic antigen-specific IgA and IgG, as demonstrated in FIGS. 20A-20D, showing IgA in the bronchiolar lavage fluid (BALF), and in the serum.

### Example 3. IM mRNA-LNP Priming Followed by IM or IN mRNA-PACE Boosting in the Context of Waning mRNA-LNP Immunity Protects Against Lethal SARS-CoV-2 Challenge

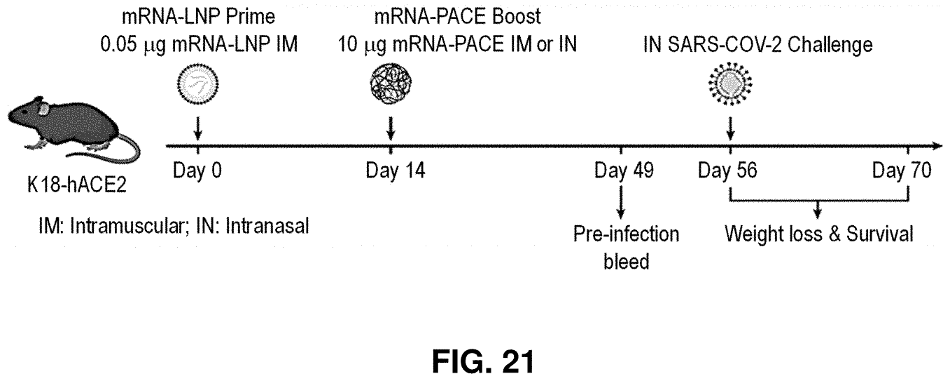

Efficacy of using mRNA-PACE as a boosting agent to increase protective immunity against an intranasal SARS-CoV-2 challenge was assessed. A schematic flow diagram for the experimental setup is set forth in FIG. 21. Briefly, antigen (mRNA-lipid nanoparticles) at 0.05 microgram RNA were injected into a K18-hACE2 mouse model via the intramuscular (IM) route at Day 0. A boost step (injecting mRNA-PACE particles at 10 microgram RNA, via intramuscular (IM) or intranasal (IN) route) was carried out at Day 14. Pre-infection bleed was carried out at Day 49. These K18-hACE2 mice were challenged with intranasal SARS-CoV-2 at Day 56. Weight loss and survival was monitored for a period of 14 days post infection from Day 56 to Day 70.

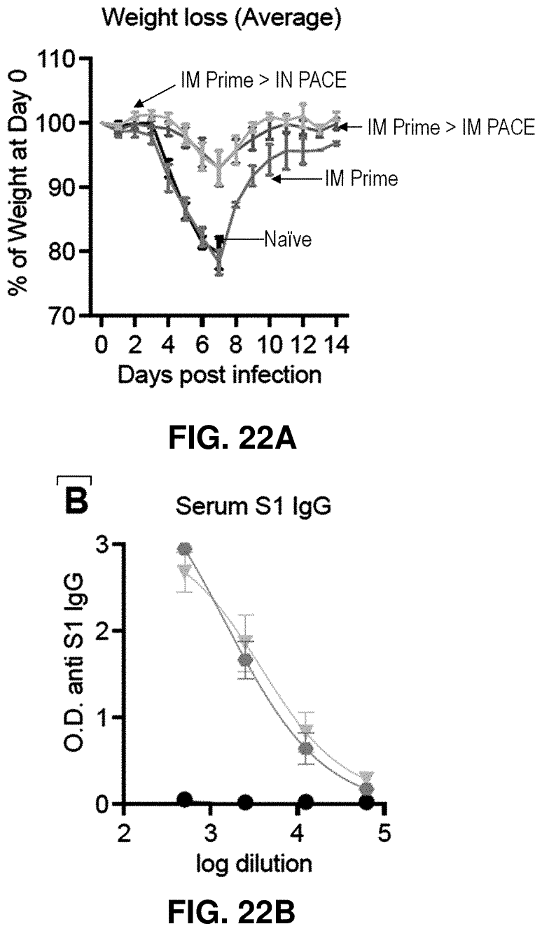

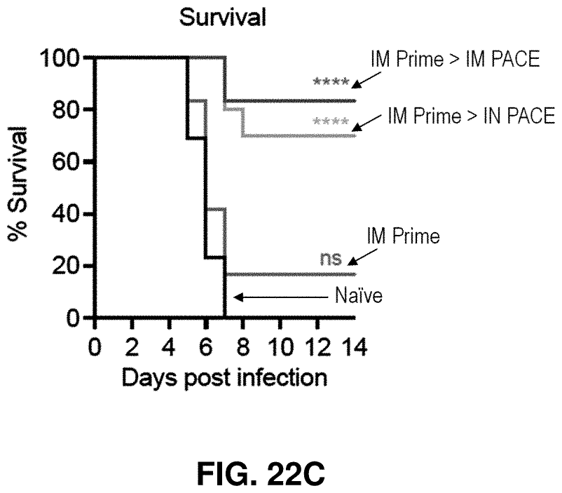

Intramuscular (IM) injection of mRNA-lipid nanoparticles (LNP), when used as a “prime” vaccination, followed by intra-nasal (IN) or IM administration of mRNA-PACE, used as a “boost” vaccination 14 days post-priming, prevented substantial weight loss and significantly increased survival after an intranasal SARS-CoV-2 challenged (FIGS. 22A-22C).

### Example 4. IN mRNA-PACE Priming Followed by IN mRNA-PACE Boosting as a Mucosal Vaccine Platform

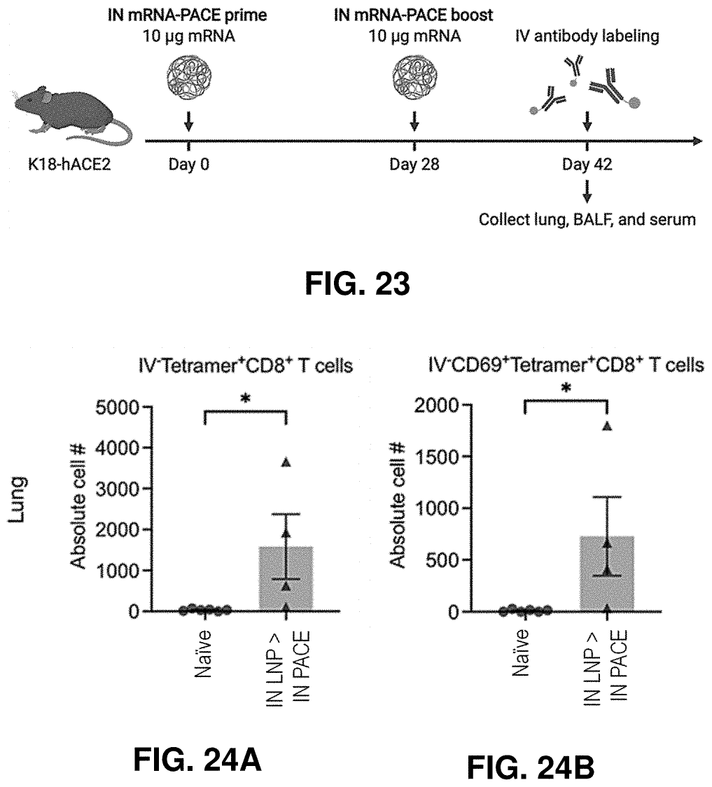

Intra-nasal (IN) administration of mRNA-PACE, when used as a “prime” vaccination, followed by intra-nasal (IN) administration of mRNA-PACE, used as a “boost” vaccination 28 days post-priming, induced tissue-resident (IV⁻) spike-specific CD8 T cell immunity and robust humoral immunity. A schematic flow diagram for the experimental setup is set forth in FIG. 23.

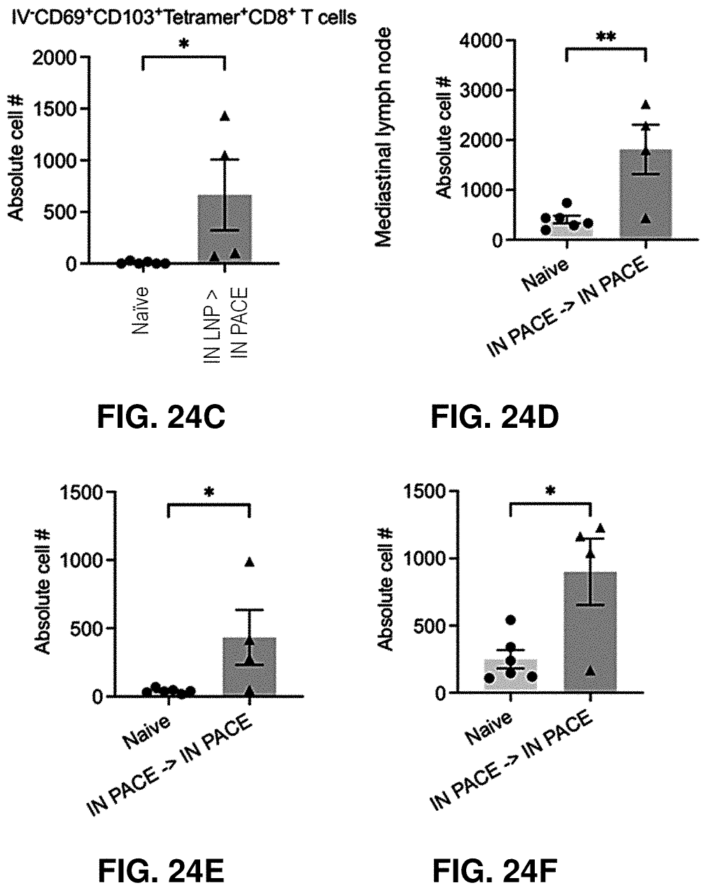

Results showing absolute cell numbers for each of samples (Naïve; IN PACE>IN PACE spike-specific CD8 T cells) are presented in FIGS. 10A-10C for “IV⁻” lung-resident T cells, and in FIGS. 24D-24F for IV⁻” mediastinal lymph node-resident T cells, respectively.

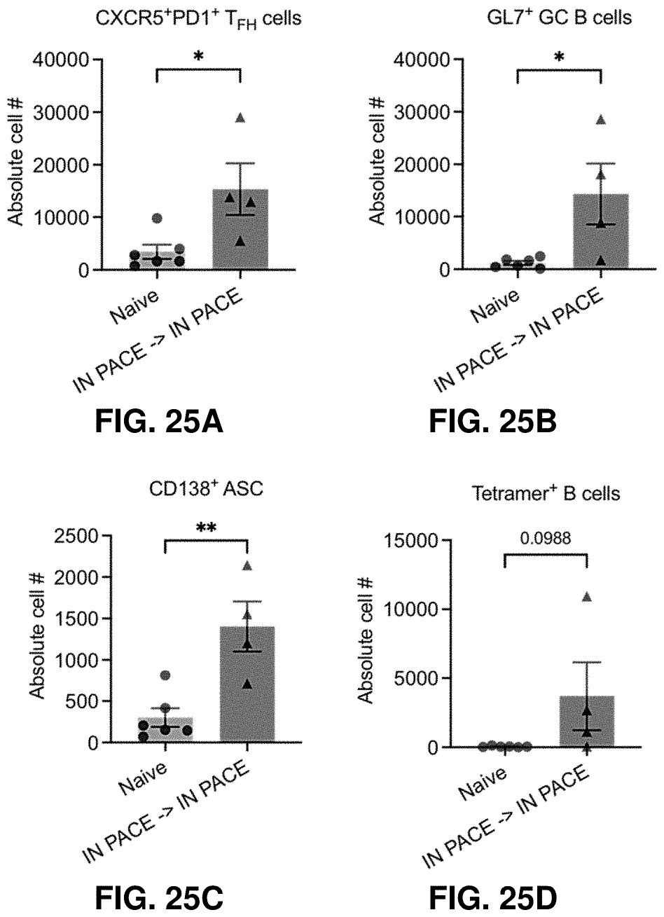

In addition, it was demonstrated that intranasal (IN) administration of mRNA-PACE, when used as a “prime” vaccination, followed by intranasal (IN) administration of mRNA-PACE, used as a “boost” vaccination 28 days post-priming, elicited robust humoral immune response in the draining lymph node. Results showing absolute cell numbers for each of samples (Naïve; IN PACE>IN PACE spike-specific CD8 T cells) are presented in FIGS. 25A-25D for CXCR5⁺PD1⁺T_{FH} cells, GL7+ germinal center (GC) B cells, CD138+ antibody secreting cells (ASC), and Tetramer+ B cells.

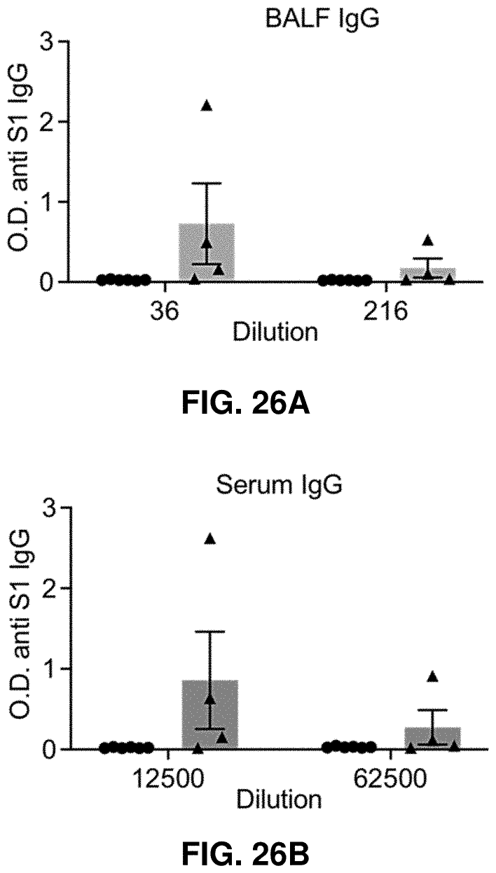

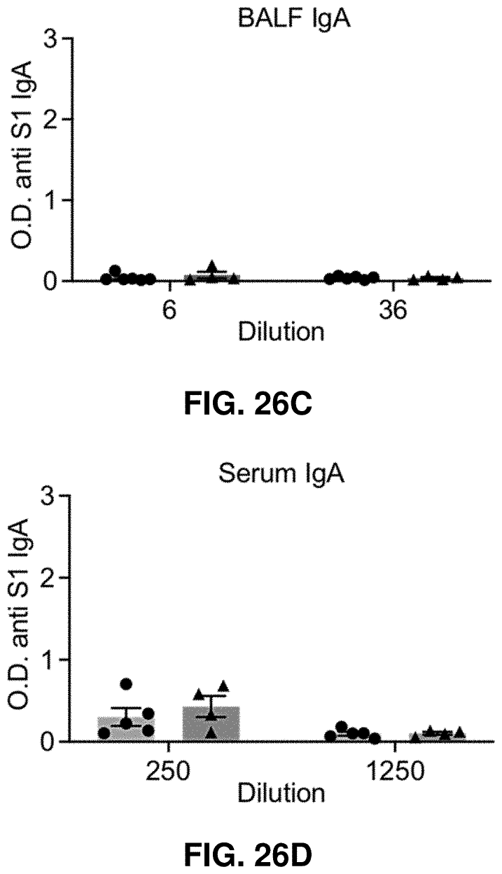

Results showing robust mucosal and systemic antigen-specific IgA and IgG, are presented in FIGS. 26A-26D, showing IgG and IgA in the bronchiolar lavage fluid (BALF), and in the serum.

### Example 5

Single-Dose Parenteral mRNA Vaccination Fails to Establish Tissue-Resident T Cell Immunity Elicited by Natural Infection

B6.Cg-Tg(K18-ACE2)₂Prlmn/J (K18-hACE2) mice either received 1 μg of Pfizer-BioNTech mRNA vaccination via intramuscular injection (Vaccine) or were infected with 500 PFU WA1 strain SARS-CoV-2 via intranasal administration (Infection). 14 days post vaccination or infection, mice were injected intravenously (IV) with anti-CD45 labeling antibodies to distinguish circulating from tissue-resident T cell responses.

Vaccination with the Pfizer-BioNTech mRNA vaccine and infection by SARS-CoV-2 virus resulted in comparable levels of viral spike-specific CD8+ T cells in the circulation. The vaccination, however; elicited significantly lower level of tissue-resident CD8+ T cells specific for the viral spike protein. Similar results were found for CD8+ memory T cells (CD69+CD103+CD8+) (data not shown).

“Prime and Spike” Vaccination Induced Tissue-Resident Immunity Against SARS-CoV-2

K18-hACE2 mice (transgenic mice expressing the human ACE2, the receptor recognized by the spike proteins of some coronaviruses, including SARS-CoV-2, for entering the host cells) were administered intramuscularly with 1 μg of an mRNA-LNP SARS-CoV2 vaccine (COMIRNATY vaccine by Pfizer-BioNTech) (“prime”) at day −14. 14 days later the mice were then administered intranasally with 1 μg of trimeric SARS-CoV2 spike protein at day 0 (“spike”). Spike protein used in this example is stabilized in its prefusion confirmation with the addition of a C-terminal T4 fibritin trimerization motif, six proline substitutions (F817P, A892P, A899P, A942P, K986P, V987P), and alanine substitutions (R683A and R685A) in the furin cleavage site (herein after, stabilized spike protein). K18-hACE2 mice that were neither primed nor spiked (“Naïve”), only primed with the intramuscular mRNA-LNP vaccine (“IM mRNA-LNP”) at the prime timepoint (day −14), or only administered with the spike protein (“IN Spike Protein”) at the boost timepoint (day 0) were used as controls. At day 14, all the mice were injected intravenously with an APC/Fire 750 CD45 antibody (30-F11, AB_2572116, BioLegend #103154) 3 minutes prior to euthanasia and biosample collection, labeling only immune cells in the vasculature but not immune cells in the tissues. The mice were then sacrificed and lungs, bronchoalveolar lavage fluid (BALF) and serum were collected.

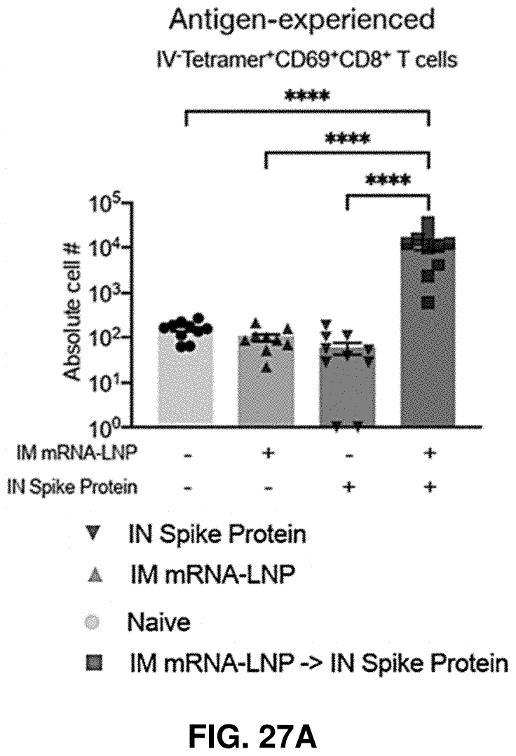

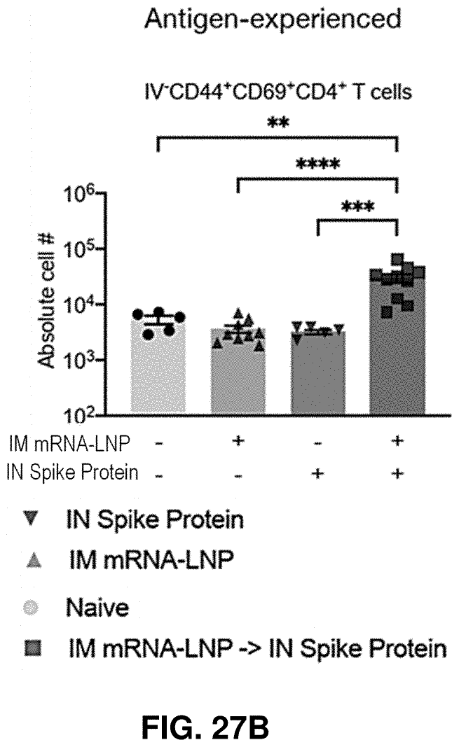

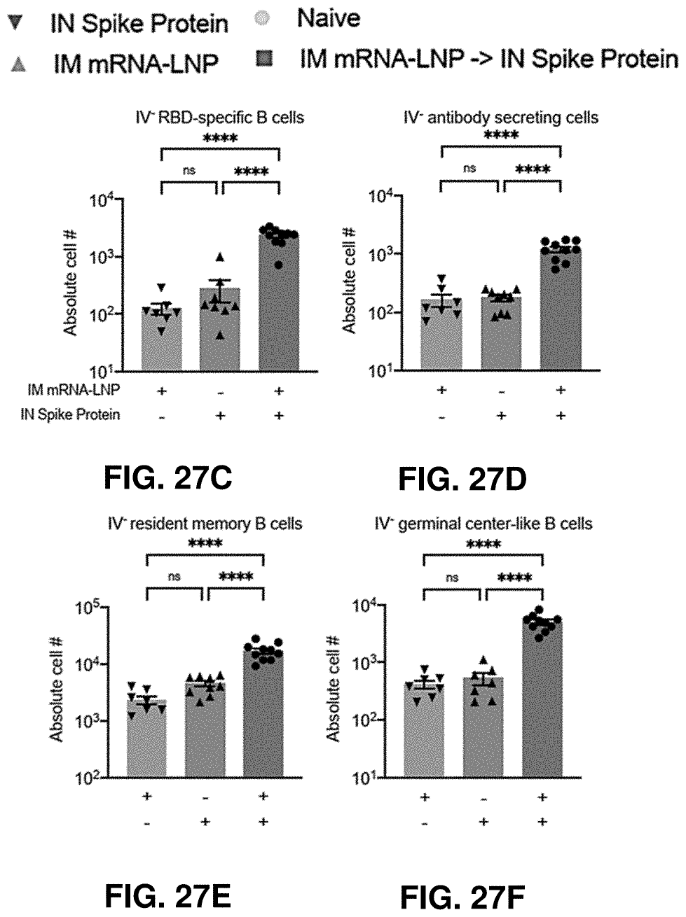

Mice that were both “primed” and “spiked,” in comparison to mice that lack “priming,” “spiking” or both, had significantly higher number of extravascular SARS-CoV-2 spike specific CD8 T cells (IV⁻Tetramer⁺CD8⁺ Cells) (data not shown, see also, FIGS. 2A, 2D and 2G), extravascular SARS-CoV-2 spike-specific antigen-experienced CD8 T cells (IV⁻Tetramer⁺CD69⁺CD8⁺ Cells) (FIG. 27A), extravascular SARS-CoV-2 spike-specific tissue-resident memory CD8 T cells (IV⁻Tetramer⁺CD103⁺CD69⁺CD8⁺ Cells) (data not shown, see also, FIGS. 2C, 2F and 2I), activated extravascular CD4 T cells (IV⁻CD44⁺CD4⁺ Cells)(data not shown, see also, FIGS. 2J and 2K), activated antigen-experienced extravascular CD4 T cells (IV⁻CD44⁺CD69⁺CD4⁺ Cells) (FIG. 27B), activated extravascular CD4 tissue-resident memory T cells (IV⁻CD44⁺CD69⁺CD103⁺CD4⁺ T Cells) (data not shown, see also, FIGS. 2L and 2O), extravascular B cells that are specific for the receptor-binding domain (RBD) of SARS-CoV-2 spike protein (IV-RBD-specific B Cells) (FIG. 27C), extravascular antibody-secreting B cells (IV-antibody secreting Cells) (FIG. 27D), extravascular resident-memory B cells (IV-resident memory B Cells) (FIG. 27E), and extravascular germinal center-like B cells ((IV-germinal center-like B Cells) (FIG. 27F.

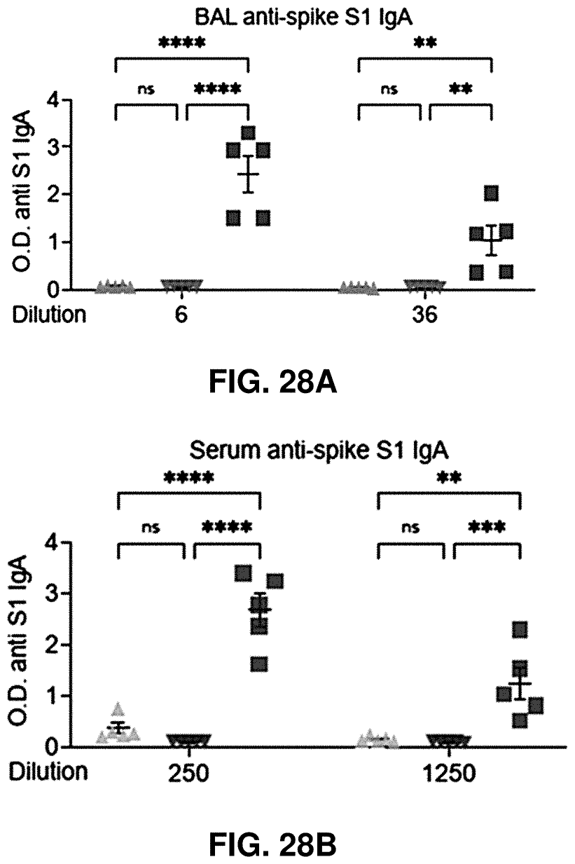

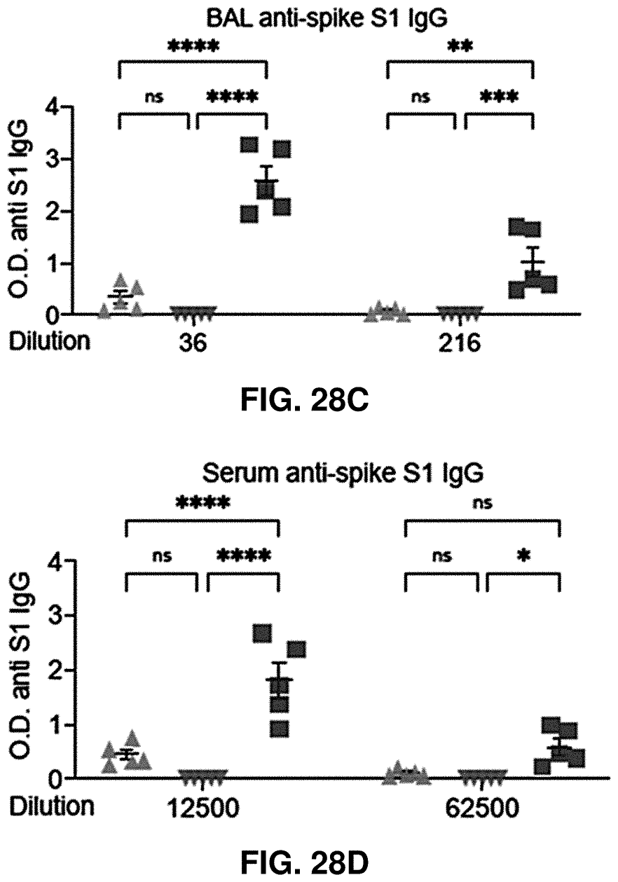

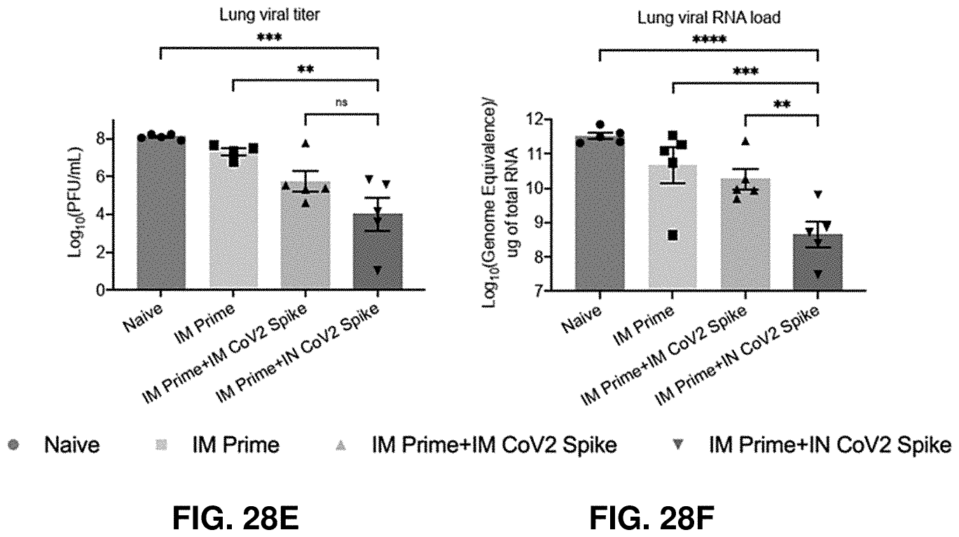

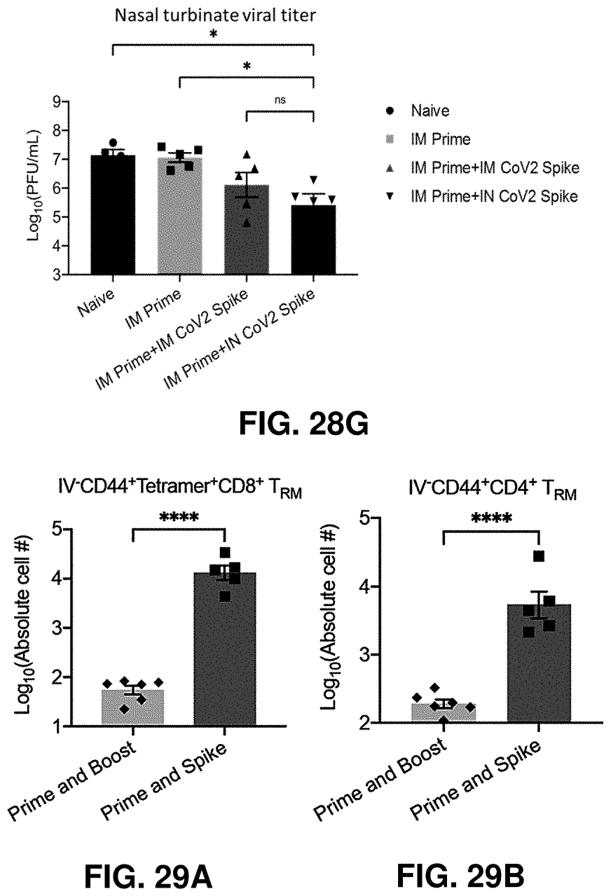

Referring to FIGS. 28A-28D, mice that were both “primed” and “spiked,” in comparison to mice that lack “priming” or “spiking,” had significantly higher concentration of anti-SARS-CoV-2 spike protein S1 domain IgA in the BALF (FIG. 28A), anti-SARS-CoV-2 spike protein S1 domain IgA in the serum (FIG. 28B), anti-SARS-CoV-2 spike protein S1 domain IgG in the BALF (FIG. 28C), and anti-SARS-CoV-2 spike protein S1 domain IgG in the serum (FIG. 28D).

Mice that were both “primed” and “spiked,” in comparison to mice that lack “priming” or “spiking,” had significantly higher numbers of SARS-CoV-2 spike-specific extravascular CD8+ T cells in the nasal turbinates (FIG. 2J) and nasal mucous (FIG. 27E), as well as significantly higher concentration of SARS-CoV-2 spike-specific IgA (FIG. 28F) and IgG (FIG. 28G) in the nasal mucous.

Mice that were both “primed” and “spiked,” in comparison to mice that lack “priming” or “spiking,” had significantly more pronounced lymphoid aggregate formations associated with vasculatures and airways. The extent of cell proliferation at the lymphoid aggregates is much higher for mice that were both “primed” and “spiked” indicating stronger proliferating immune responses in these mice. B cells, CD8 T cells and CD4 T cells concentrate at the lymphoid aggregates (data not shown).

“Prime and Spike” Vaccination Induced Stronger Mucosal T Cell and Antibody Responses than Conventional “Prime and Boost” Vaccination

C57BL/6J mice were administered intramuscularly with an mRNA-LNP SARS-CoV-2 vaccine (COMIRNATY) at Day −14 (“prime”). The mice were then either administered intramuscularly with 1 μg of a conventional mRNA-LNP SARS-CoV-2 vaccine booster (“boost”) (COMIRNATY) or administered intranasally with 1 μg of SARS-CoV-2 stabilized spike protein (“spike”) at day 0. At day 7, the mice were sacrificed and lungs and BALF were collected.

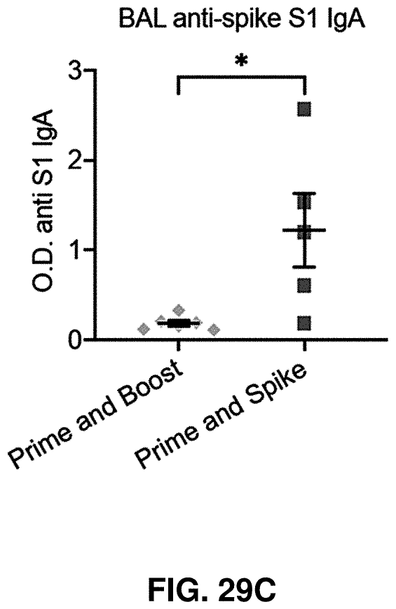

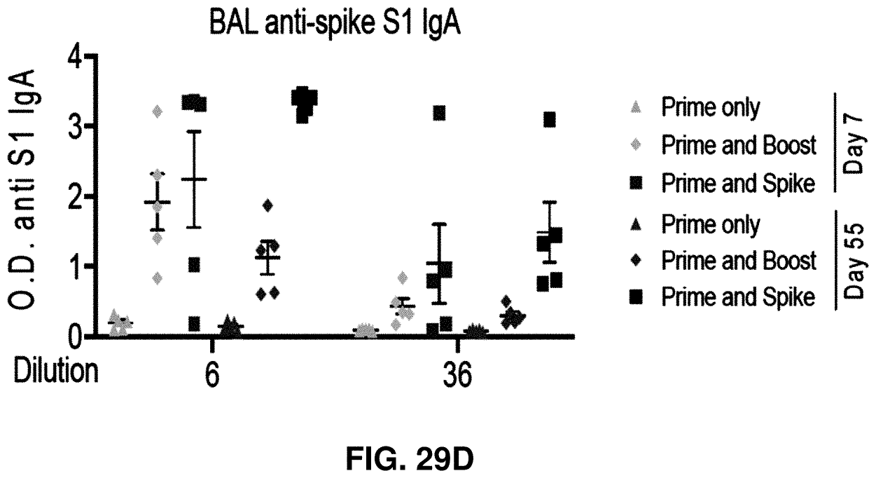

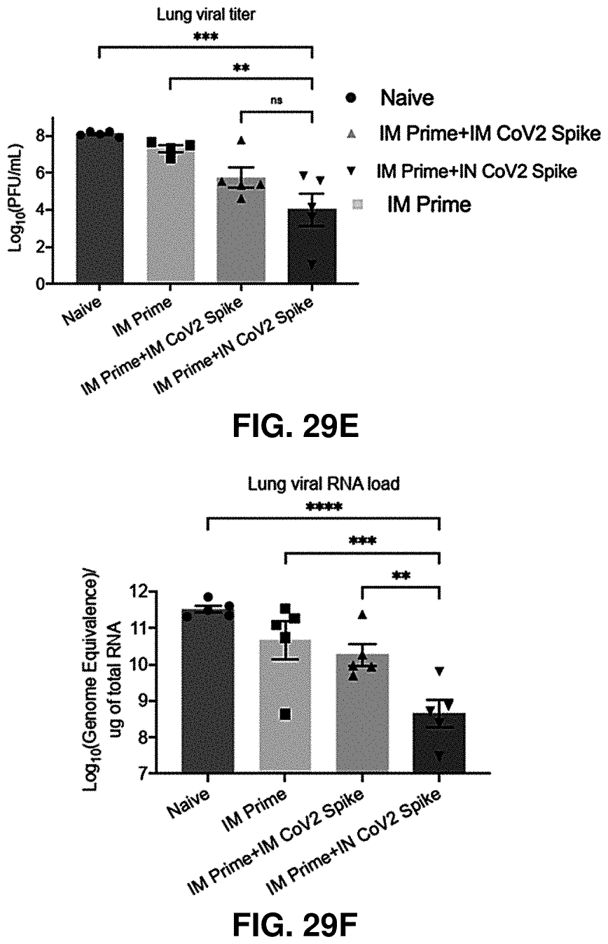

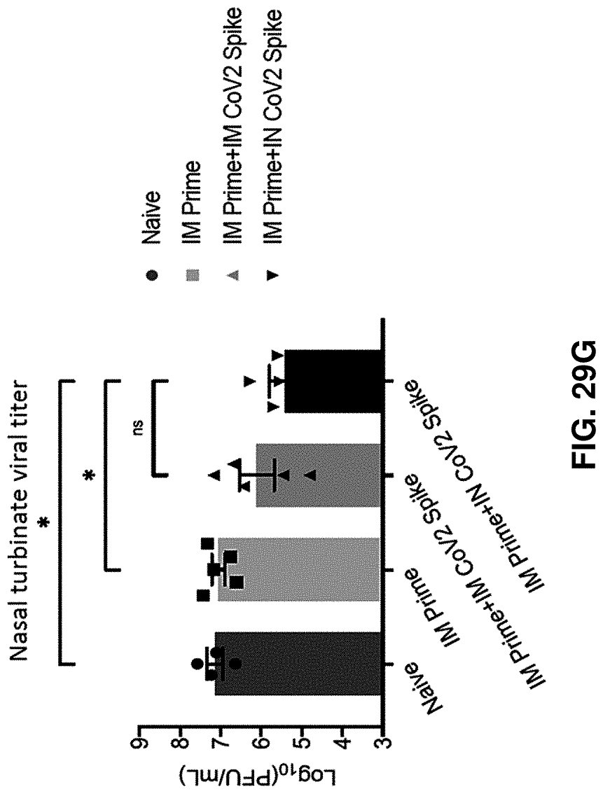

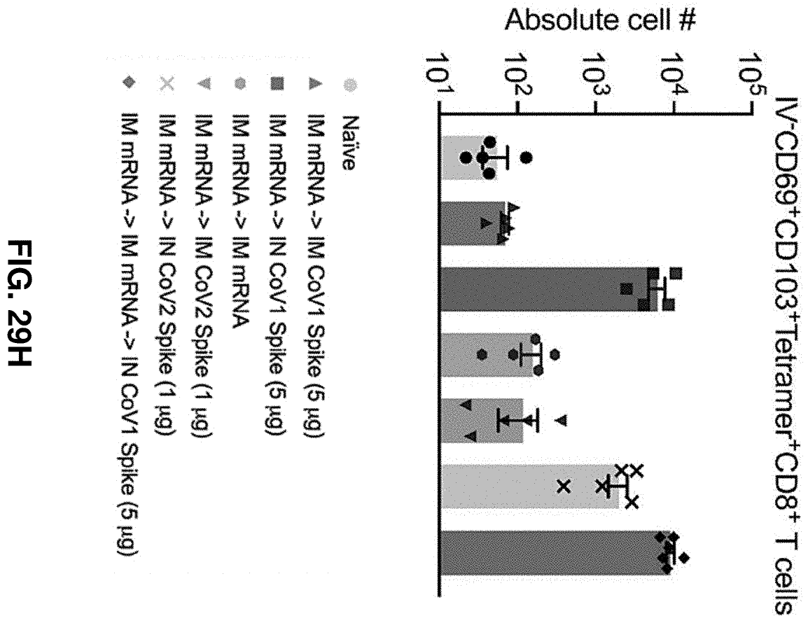

Referring to FIGS. 29A-29C, mice administered with the “prime and spike” vaccination, in comparison with mice administered with the conventional “prime and boost” vaccination, had significantly higher numbers of activated lung-resident memory CD8 T cells (IV⁻CD44⁺Tetramer⁺CD8⁺ T_{RM} (FIG. 29A) and activated lung-resident memory CD4 T cells (FIG. 29B). Mice administered with the “prime and spike” also had much higher anti-SARS-CoV-2 spike protein S1 domain IgA in the BALF than mice administered with the conventional “prime and boost” vaccination.

“Prime and Spike” Vaccination Induces Stronger and Longer Lasting Immunity than the Conventional “Prime and Boost” Vaccination when Following Extended Between-Dose Intervals

K18-hACE2 mice were administered intramuscularly with an mRNA-LNP SARS-CoV-2 vaccine (COMIRNATY) at Day −84 (“primed”). At Day 0, the mice were administered either intramuscularly with the same SARS-CoV-2 vaccine (“boosted”) or intranasally with the recombinant stabilized spike protein of SARS-CoV-2 (“spiked”). The mice were sacrificed at either Day 7 or Day 55, and BALF and serum were collected.

Referring to FIG. 29D, the mucosal IgA response induced by the “prime and spike vaccination” strategy was both stronger and longer lasting than the conventional “prime and boost” vaccination strategy.

“Prime and Spike” Vaccination Induced Superior Anti-Viral Protection Against SARS-CoV-2 Infection in Comparison to the Conventional “Prime and Boost” Vaccination

K18-hACE2 mice were administered intramuscularly with 0.05 μg of an mRNA-LNP SARS-CoV-2 vaccine (COMIRNATY) at day −14 (“prime”). The mice were then administered intranasally (IN) with 1 μg of the trimeric SARS-CoV-2 stabilized spike protein at day 0 (“spike”). K18-hACE2 mice that lacked both “priming” and “spiking” (“naïve”), lacked only “spiking” (“IM Prime”), or were administered with 1 μg of the SARS-CoV-2 spike protein intramuscularly instead of “spiked” (“IM Prime+IM CoV-2 Spike”) were used as controls. All mice were challenged with live SARS-CoV-2 virus at day 42. The survival and weight loss of the mice were recorded from day 42 to day 56. Lung viral titer and lung viral RNA load were also measured at day 44.

Mice that were both “primed” and “spiked,” in comparison with controls, suffered from less weight and were fully protected against lethality of the virus (data not shown).

Referring to FIGS. 29E-29G, mice that were both “primed” and “spiked,” in comparison with controls, had lower amount of virus in the lungs (lower respiratory tract) and the nasal turbinates (upper respiratory tract), as determined by viral titer (FIGS. 29E and 29G) and viral RNA load in the lungs (FIG. 29E

“Priming” and “Spiking” Using Antigens from Two Related but Different Viruses is Able to Generate Robust Immunity Against Both Viruses

Mice were “primed” by administering intramuscularly with 1 μg of the mRNA-LNP SARS-CoV2 vaccine (COMIRNATY) at day 0. 14 days later the mice were “boosted” with 1 μg of trimeric SARS-CoV-2 (SCV2) stabilized spike protein intramuscularly (IM) or “spiked” intranasally (IN) with the same, “boosted” with 5 μg of trimeric SARS-CoV-1 (SCV1) stabilized spike protein or “spiked” with the same, or “boosted” with 1 μg of an mRNA-LNP SCV2 vaccine IM, at day 14. At the time of boosting (day 14), K18-hACE2 mice that were previously primed (at day −53) and boosted (at day −16) with mRNA-LNP SCV2 vaccine IM were also re-boosted with 5 μg of trimeric SCV1 stabilized spike protein IN.

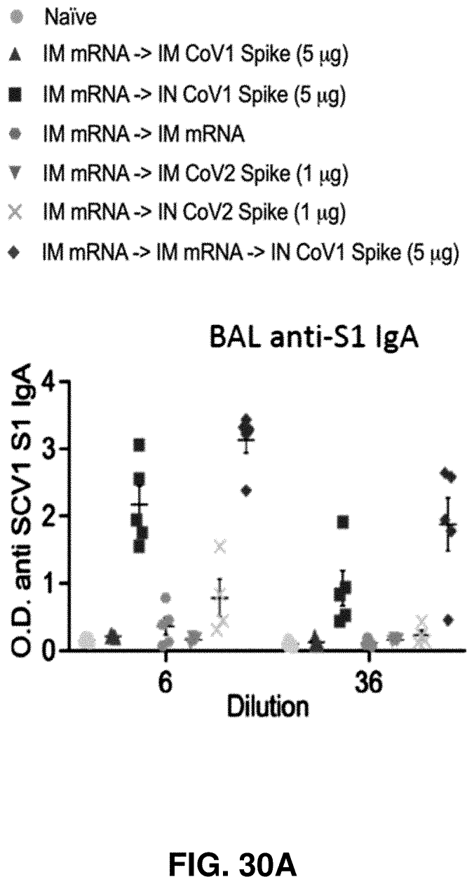

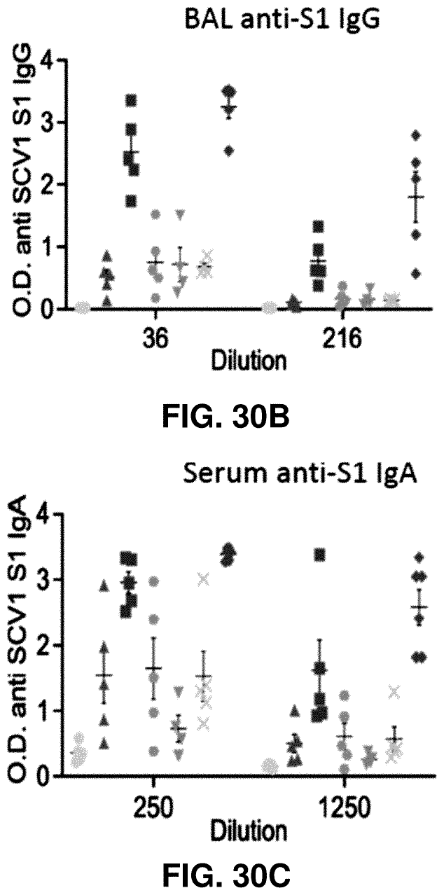

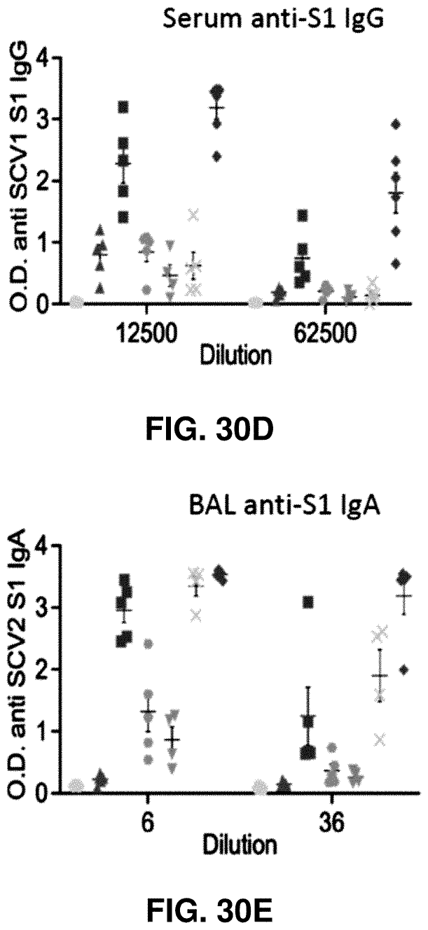

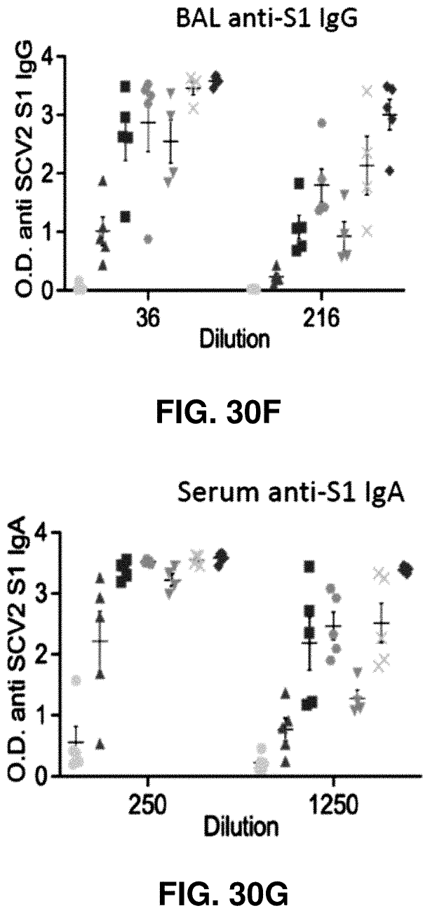

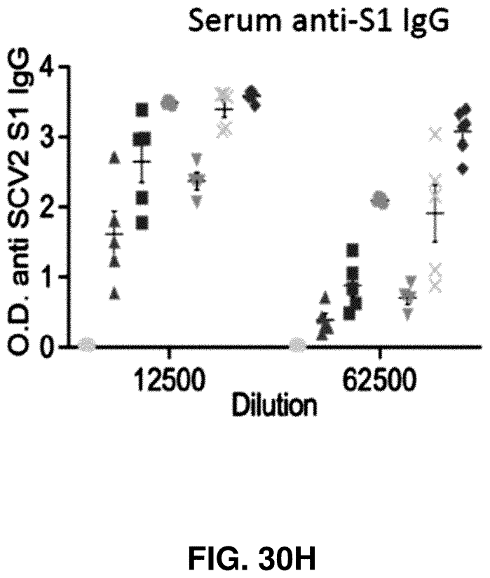

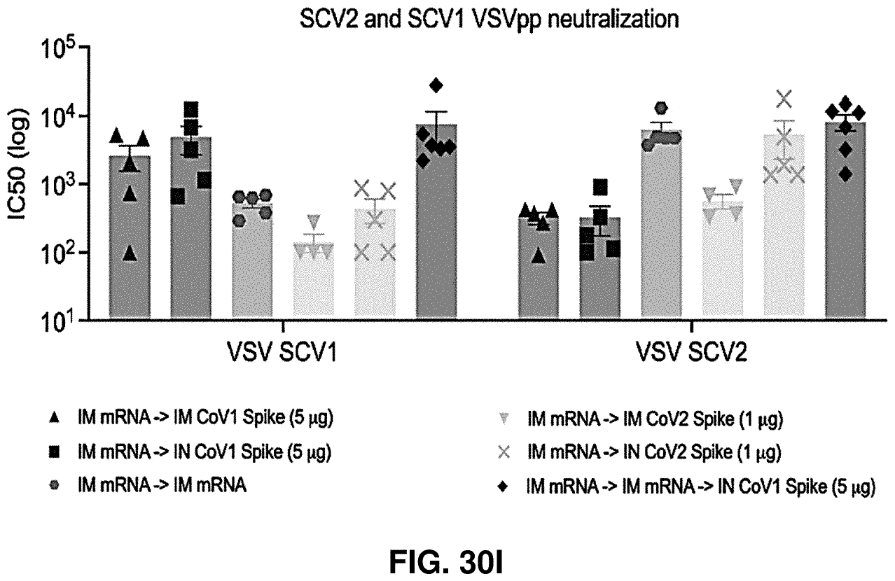

K18-hACE2 mice that were neither primed nor boosted (“Naïve”) were used as controls. At day 45, all the mice were injected intravenously with an APC/Fire 750 CD45 Ab (30-F11, AB_2572116, BioLegend #103154) 3 minutes prior to euthanasia and sample collection, labeling only immune cells in the vasculature but not immune cells in the tissues. Mice were then sacrificed and lungs, bronchoalveolar lavage fluid (BALF) and serum were collected. Lung tissues were processed for assessment of CD8 T cell responses (FIG. 29H), and BALF and serum were used to assess anti-SCV1 and anti-SCV2 binding antibody (FIGS. 30A-30D and 30EA-30H) and neutralizing antibody (FIG. 30I) responses.

Referring to FIG. 29G, mice that were “primed” intramuscularly and “spiked” intranasally using either SCV2 or SCV1 spike proteins developed significantly higher number of tissue-resident memory CD8+ T cells specific for a conserved epitope shared between SCV1 and SCV2 (VNFNFNGLSEQ ID NO:6), compared to naïve mice as well as mice that were “primed” intramuscularly and “boosted” intramuscularly with SCV2 spike protein, SCV1 spike protein, or the mRNA-LNP SCV2 vaccine. Mice that were “primed” and “boosted” with the mRNA-LNP SCV2 vaccine and additionally “spiked” intranasally with SCV1 spike protein also developed high levels of antigen-specific tissue-resident memory CD8 T+ cells specific for a conserved epitope shared between SCV1 and SCV2 (VNFNFNGL, SEQ ID NO:6).

Referring to FIGS. 30A-30D, mice that were IM “primed” and IN “spiked” with SARS-CoV (SCV1) spike proteins had significantly higher titers of anti-SCV1 IgA and IgG in both BALF and serum compared to mice that were IM “primed” and later “boosted” with IM mRNA-LNP SCV2 vaccine, IM SCV1 spike protein, or IM SCV2 spike, or “spiked” IN with SCV2 spike protein. Mice that were intramuscularly “primed and boosted” with mRNA-LNP SCV2 vaccine and additionally intranasally “spiked” with SCV1 spike protein had further increases of mucosal and circulating anti-SCV1 IgA and IgG compared to mice that were IM “primed” and IN SCV1 “spiked.”

Referring to FIGS. 30E-30H, mice that were intramuscularly “primed” and intranasally “spiked” with SARS-CoV (SCV1) or SARS-CoV-2 (SCV2) spike proteins had significantly higher titers of anti-SCV2 IgA in the BALF compared to mice that were IM “primed” and later “boosted” with IM mRNA-LNP SCV2 vaccine, IM SCV1 spike, or IM SCV2 spike. Notably, mice that were IM “primed” and IN SCV2 “spiked” developed comparable levels of BALF anti-SCV2 IgG, serum anti-SCV2 IgA, and serum anti-SCV2 IgG compared to mice that were IM “primed” and “boosted” with mRNA-LNP SCV2 vaccine. Mice that were IM “primed” and IN SCV1 “spiked” also developed robust levels of BALF anti-SCV2 IgG, serum anti-SCV2 IgA, and serum anti-SCV2 IgG. Similar to anti-SCV1 antibody response, mice that were IM “primed” and “boosted” with mRNA-LNP SCV2 vaccine and additionally IN “spiked” with SCV1 spike protein developed highest levels of mucosal and circulating anti-SCV2 IgA and IgG compared to all other groups.

Referring to FIG. 30I, mice that were IM “primed” and later “spiked” with IN SCV1 or IM SCV1 spike proteins had significantly higher serum neutralizing titers (IC50) against Vesicular Stomatitis Virus (VSV)-pseudotyped SCV1 compared to mice that were IM “primed” and later “boosted” with IM mRNA-LNP SCV2 vaccine, IM SCV2 spike, or “spiked” with IN SCV2 spike protein. Mice that were IM “primed” and later “spiked” with IN SCV2 spike or “boosted” with IM mRNA-LNP SCV2 vaccine had significantly higher serum neutralizing titers (IC50) against VSV-pseudotyped SCV2 compared to mice that were IM “primed” and later “boosted” with IM SCV2 spike protein, IM SCV1 spike protein, or “spiked” with IN SCV1 spike protein. Consistent with binding antibody titers, mice that were IM “primed” and “boosted” with mRNA-LNP SCV2 vaccine and additionally IN “primed” with SCV1 spike developed high serum neutralizing titers (IC50) against both VSV-pseudotyped SCV1 and SCV2.

Discussion

This work describes the preclinical development of alternative vaccine strategies (i) prime and spike (P&S), (ii) prime and PACE-spike); and (iii) PACE-prime and PACE-spike.

In P&S, IN unadjuvanted spike subunit protein elicits robust protective mucosal immunity after mRNA-LNP parenteral immunization (prime). These enhanced mucosal responses are characterized by the expansion of antigen-specific CD8⁺T_{RM}, CD4⁺T_{RM}, and B_{RM} cells as well as mucosal secretion of IgA and IgG. The data showed that an IN unadjuvanted spike booster can be administered months out from primary immunization and that it offers systemic neutralizing antibody booster responses comparable with that of IM mRNA-LPN boost. Similarly, prime and PACE-spike elicits increased antigen-specific CD8⁺ T_{RM} cells and mucosal IgA. Both boosting methods result in protection from lethal SARS-CoV-2 challenge. The data also showed that P&S leads to durable responses with protective vaccine efficacy at 118 days from the initiation of vaccination. P&S/PACE-prime and PACE-spike is protective in hamsters and blocks viral transmission more effectively than does mRNA-LNP prime-boost. By using a divergent spike antigen, the present study demonstrate that P&Sx can generate mucosal immunity to SARS-CoV-1 while also boosting the systemic and mucosal neutralizing anti-bodies to the original antigenic target, SARS-CoV-2. Although the goal of vaccination has been to prevent individual morbidity and mortality, the evolution of SARS-CoV-2 has high-lighted the need for rapidly deployable mucosal vaccines that also prevent transmission. The data demonstrates that P&S, prime and PACE-spike, PACE-prime and PACE-spike can be used in reducing both infection and transmission. Improving upon current vaccine platforms to provide mucosal immunity is vital to control this pandemic and will certainly be important for the next.

P&S, prime and PACE-spike, PACE-prime and PACE-spike are a broadly applicable as a booster against new SARS-CoV-2 VOCs in a previously vaccinated individual or as a de novo primary immunization strategy for newly emerging respiratory pathogens. Although the present studies demonstrate the vaccine regiment using of mRNA-LNP priming, this approach can be extrapolated to primary immunization regimens or in the case of previous infection. Further, the data presented here supports a conclusion that unadjuvanted IN boosting would be as effective if not more so in individuals who have received multiple previous shots because P&S/PACE-spike, seem to leverage pre-existing immunity rather than be inhibited by it. Additionally, it has been shown that the highly stabilized spike enhances its immunogenicity and that applying this vaccination strategy to other pathogens may require the addition of stabilizing mutations to enable un-adjuvanted boosting.

The present study characterizes a method for the development of mucosal immunity to SARS-CoV-2 without the use of adjuvants or replicating viruses or vectors in two different well-validated preclinical vaccine models.

Vaccines that generate broadly neutralizing immunity against a wide variety of sarbeco viruses are a goal to combat both newly emerging SARS-CoV-2 variants and potential pandemic SARS-like coronaviruses. Using SARS-CoV-1 spike as a heterologous IN boost, P&Sx demonstrates that prior SARS-CoV-2 mRNA-LNP does not prevent the development of SARS-CoV-1-neutralizing antibodies but rather enables it. P&Sx simultaneously elicits broadly reactive neutralizing antibodies and mucosal immunity.

Modifications and variations of the method and compositions described herein are intended to come within the scope of the following claims. All references cited herein are specifically incorporated by reference.

We claim:

1. A method of enhancing mucosal immunity to an antigen in a subject, the method comprising
administering to a mucosal tissue in the subject an effective amount of a vaccine composition comprising a polynucleotide selected from the group consisting of messenger RNA (mRNA), deoxyribonucleic acid (DNA), ribonucleic acid (RNA), and other polynucleotides molecules encoding a viral or cancer cell antigen, in a carrier,
wherein the carrier comprises poly(amine-co-ester) (PACE) polymers, poly(amine-co-ester)s polymers, or poly(amine-co-amide)s polymers or is a carrier material selected from the group consisting of liposomes, liposome-like particles, solid lipid particles, nanostructured lipid carriers, absorbed lipid-polymer hybrid particles, encapsulated lipid-polymer hybrid particles, cation nanoemulsions, exosomes, native lipoprotein, and synthetic lipoprotein.

2. The method of claim 1 comprising the steps of:
(i) administering via a systemic or mucosal route of administration an effective amount of the composition to prime an immune response to the antigen; and
(ii) subsequently administering to a mucosal surface an effective amount of the composition to increase or boost the immune response to the antigen.

3. The method of claim 2, wherein the priming antigen shares one or more antigenic sites with the boosting antigen.

4. The method of claim 1, wherein the method induces an enhanced number of cells selected from the group consisting of CD8⁺ tissue-resident memory (T_{RM}) cells, CD4⁺ tissue-resident memory (T_{RM}) cells, and memory B cells against the antigen, at the mucosal tissue in the subject compared to the cell number prior to the step of administering to the mucosal tissue, or wherein the method induces a higher titer of mucosal IgA compared to the IgA titer prior to the step of administering to the mucosal tissue.

5. The method of claim 1, wherein the mucosal tissue is one or more tissues selected from the group consisting of pulmonary, nasal, oral, gastrointestinal, vaginal, and rectal mucosa.

6. The method of claim 1, wherein the antigen is derived from, or raises a protective immune response against, one or more respiratory viruses selected from the group consisting of orthomyxoviruses, paramyxoviruses, coronaviruses, adenoviruses, herpesviruses, and human bocaviruses.

7. The method of claim 1, wherein the antigen is derived from, or raises a protective immune response against one or more respiratory viruses selected from the group consisting of influenza viruses, parainfluenza viruses, measles viruses, mumps viruses, and respiratory syncytial virus, human metapneumovirus, severe acute respiratory syndrome (SARS) virus, herpes simplex virus (HSV), cytomegalovirus (CMV), Epstein-Barr virus (EBV), and varicella-zoster virus (VZV).

8. The method of claim 6, wherein the antigen is derived from, or raises a protective immune response against an influenza virus.

9. The method of claim 8, wherein the antigen is derived from, or raises a protective immune response against a SARS-CoV-2 virus, optionally, wherein the antigen is derived from a coronavirus spike protein, wherein the coronavirus is a variant of SARS-CoV-2.

10. The method of claim 1, wherein the polynucleotide is associated with or encapsulated within a carrier material selected from the group consisting of liposomes, liposome-like particles, solid lipid particles, nanostructured lipid carriers, absorbed lipid-polymer hybrid particles, encapsulated lipid-polymer hybrid particles, cation nanoemulsions, exosomes, native lipoprotein, and synthetic lipoprotein.

11. The method of claim 1, wherein the carrier comprises poly(amine-co-ester) polymers or poly(amine-co-amide)s polymers, and pegylated polymers thereof.

12. The method of claim 11 comprising a polymer having a structure of:
(i) Formula I: 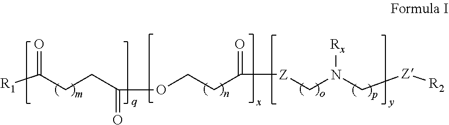
wherein R₁ and R₂ are absent or are chemical entities containing a hydroxyl group, a primary amine group, a secondary amine group, a tertiary amine group, or combinations thereof, or
(ii) Formula II: 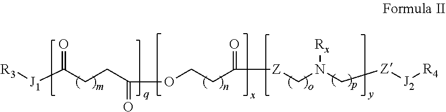
wherein J₁ and J₂ are independently linking moieties or absent, or
(iii) Formula III: 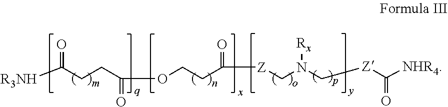
wherein for Formula II and Formula III, R₃ and R₄ are independently substituted alkyl containing a hydroxyl group, a primary amine group, a secondary amine group, a tertiary amine group, or combinations thereof,
wherein for Formula I, Formula II, and Formula III, n is an integer from 1-30, m, o, and p are independently integers from 1-20, x, y, and q are independently integers from 1-1000, Rx is hydrogen, substituted or unsubstituted alkyl, or substituted or unsubstituted aryl, or substituted or unsubstituted alkoxy, Z and Z′ are independently 0 or NR′, wherein R′ is hydrogen, substituted or unsubstituted alkyl, or substituted or unsubstituted aryl.

13. The method of claim 11 wherein the weight average molecular weight of the polymer is greater than 20,000 Daltons, greater than 15,000 Daltons, greater than 10,000 Daltons, greater than 5,000 Daltons, or greater than 2,000 Daltons.

14. A composition for inducing and/or boosting immunity against viral or cancer cell antigens in a subject, the composition comprising:
a polynucleotide encoding a viral or cancer cell antigen in a carrier selected from the group consisting of poly(amine-co-ester) (PACE) particles, liposomes, liposome-like particles, solid lipid particles, nanostructured lipid carriers, absorbed lipid-polymer hybrid particles, encapsulated lipid-polymer hybrid particles, cation nanoemulsions, exosomes, native lipoprotein, and synthetic lipoprotein.

15. The composition of claim 14, wherein the virus comprises a coronavirus, and wherein the coronavirus is at least one virus selected from the group consisting of an Alphacoronavirus, a Betacoronavirus, a Gammacoronavirus, and a Deltacoronavirus.

16. The composition of claim 14, wherein the composition does not comprise an adjuvant other than the carrier.

17. The composition of claim 14 comprising antigen selected from the group consisting of a mRNA-lipid particle (LNP)-based antigen, a viral vector antigen, an inactivated virus, a viral subunit/peptide antigen, a virus like particle (VLP)-based vaccine, and a DNA viral antigen.

18. A composition for inducing and boosting immunity against viral or cancer cell antigens in a subject, the composition comprising:
a polynucleotide encoding a viral or cancer cell antigen in poly(amine-co-ester) particles.

19. The composition of claim 18, wherein the PACE polymer comprises a polymer of Formula I: 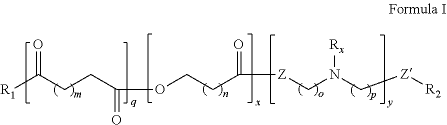
wherein n is an integer from 1-30, m, o, and p are independently integers from 1-20, x, y, and q are independently integers from 1-1000, Rx is hydrogen, substituted or unsubstituted alkyl, or substituted or unsubstituted aryl, or substituted or unsubstituted alkoxy, Z and Z′ are independently 0 or NR′, wherein R′ is hydrogen, substituted or unsubstituted alkyl, or substituted or unsubstituted aryl,
wherein R₁ and R₂ are absent or are chemical entities containing a hydroxyl group, a primary amine group, a secondary amine group, a tertiary amine group, or combinations thereof.

20. The composition of claim 19, wherein R₁ and/or R₂ are not

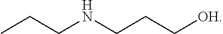

21. The composition of claim 18, wherein the polymer has a structure of Formula II: 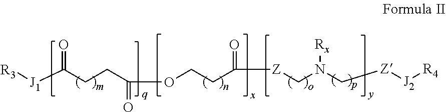
wherein J₁ and J₂ are independently linking moieties or absent,
wherein n is an integer from 1-30, m, o, and p are independently integers from 1-20, x, y, and q are independently integers from 1-1000, Rx is hydrogen, substituted or unsubstituted alkyl, or substituted or unsubstituted aryl, or substituted or unsubstituted alkoxy, Z and Z′ are independently 0 or NR′, wherein R′ is hydrogen, substituted or unsubstituted alkyl, or substituted or unsubstituted aryl,
R₃ and R₄ are substituted alkyl containing a hydroxyl group, a primary amine group, a secondary amine group, a tertiary amine group, or combinations thereof.

22. The composition of claim 21, wherein the polymer has a structure of Formula III:

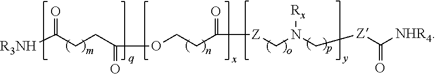

23. The composition of claim 22, wherein: (a) Z is the same as Z′; (b) n is 4, 10, 13, or 14; (c) m is 5, 6, or 7; or (d) Rₓ is substituted or unsubstituted alkyl.

24. The composition of claim 18, wherein the weight average molecular weight, as measured by gel permeation chromatography using narrow polydispersity polystyrene standards, is between about 2,000 Daltons and 20,000 Daltons, preferably between about 2,000 Daltons and about 10,000 Daltons, most preferably between about 2000 Daltons and about 7,000 Daltons.

25. The method of claim 21, wherein R₃ is the same as R₄.

26. The method of claim 21, wherein R₃ and/or R₄ are independently selected from the group consisting of

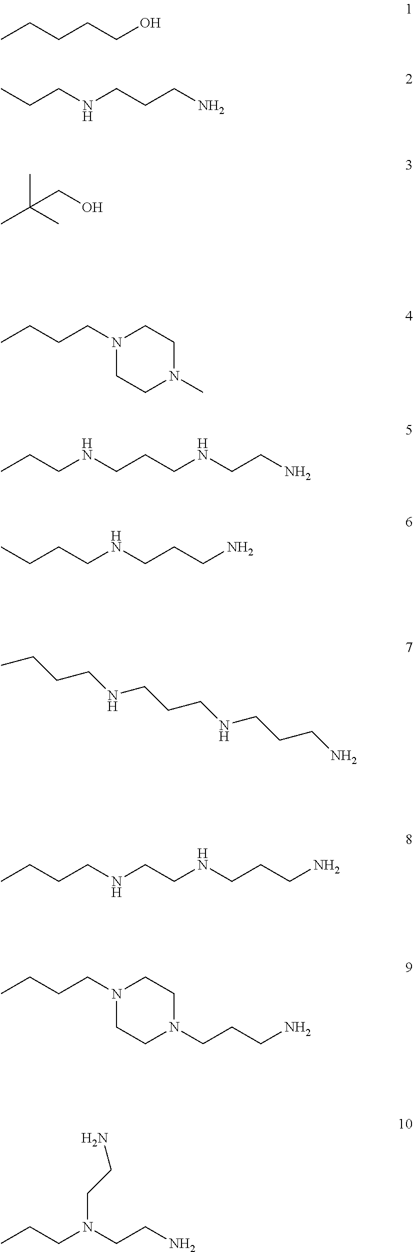

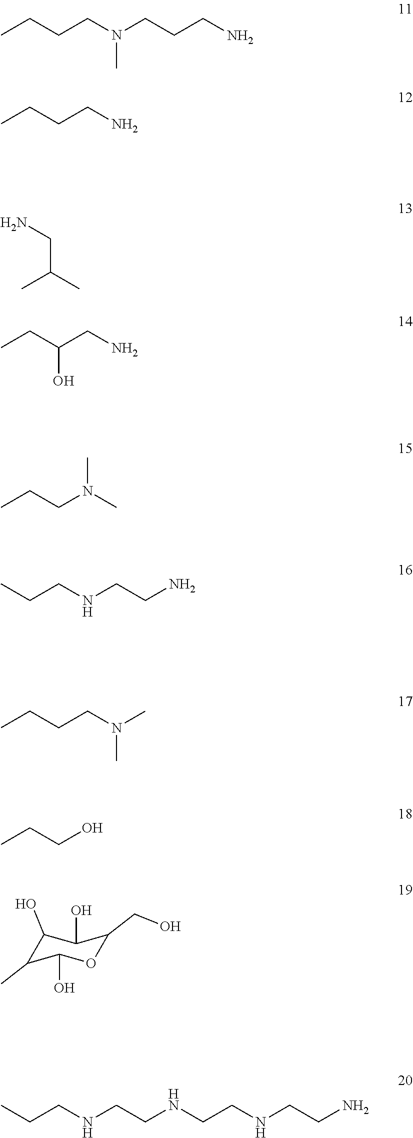

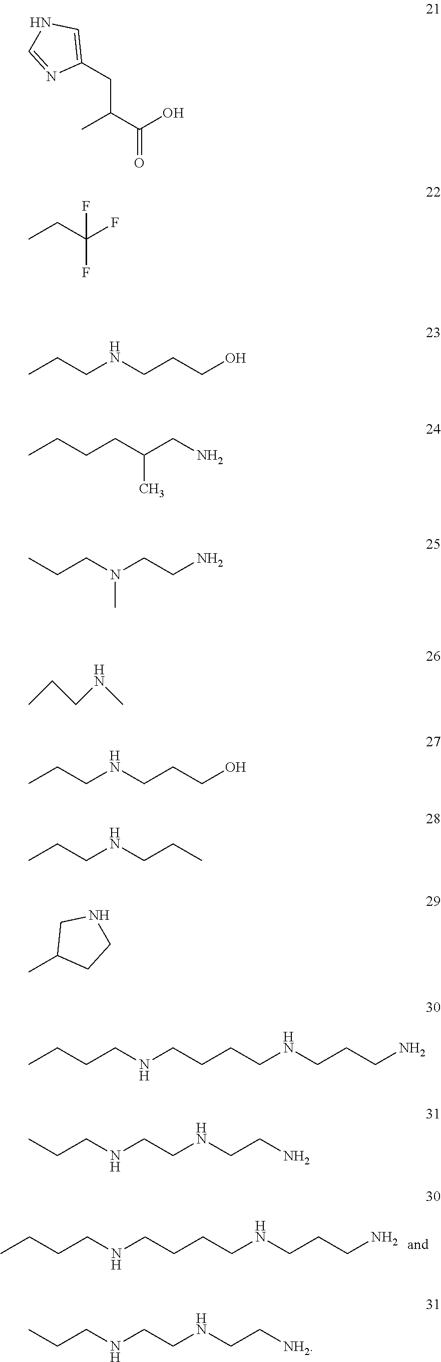

27. The composition of claim 21, wherein: (a) J₁ is —O— or —NH, J₂ is —C(O)NH— or —C(O)O— or a combination thereof; (b) R₃, R₄ or both contain a primary amine group, and optionally one or more secondary or tertiary amine groups; (c) R₃, R₄ or both contain a hydroxyl group, and optionally one or more amine groups; (d) R₃, R₄ or both contain a hydroxyl group and no amine group or (e) at least one of R₃ and R₄ does not contain a hydroxyl group.

28. The composition of claim 21, wherein R₃, R₄ or both are -unsubstituted C₁-C₁₀ alkylene-Aq-unsubstituted C₁-C₁₀ alkylene-Bq, -unsubstituted C₁-C₁₀ alkylene-Aq-substituted C₁-C₁₀ alkylene-Bq, -substituted C₁-C₁₀ alkylene-Aq-unsubstituted C₁-C₁₀ alkylene-Bq, or -substituted C₁-C₁₀ alkylene-Aq-substituted C₁-C₁₀ alkylene-Bq, wherein Aq is absent or —NR₅—, and Bq is hydroxyl, primary amine, secondary amine, or tertiary amine, wherein R₅ is hydrogen, substituted or unsubstituted alkyl, or substituted or unsubstituted aryl.

29. The composition of claim 18, wherein the polymer is prepared from one or more lactones, one or more amine-diols (Z and Z′═O) or triamines (Z and Z′ ═NR′), and one or more diacids or diesters, wherein when two or more different lactone, diacid or diester, and/or triamine or amine-diol monomers are used, the values of n, o, p, and/or m can be the same or different.

30. The composition of claim 18 comprising a PEG-conjugated poly(amine-co-ester)s or poly(amine-co-amide)s having a structure of Formula XI: 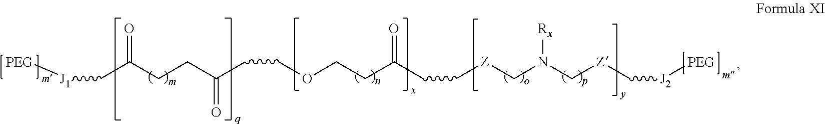
wherein m′ and m″ are independently 0 or 1, with the proviso that m′+m″ is 1 or 2,
wherein n is an integer from 1-30, m, o, and p are independently integers from 1-20, x, y, and q are independently integers from 1-1000, Rx is hydrogen, substituted or unsubstituted alkyl, or substituted or unsubstituted aryl, or substituted or unsubstituted alkoxy, Z and Z′ are independently 0 or NR′, wherein R′ is hydrogen, substituted or unsubstituted alkyl, or substituted or unsubstituted aryl,
J₁ and J₂ in Formulae XI are independently absent or linking moieties such as —C(O)—, —C(O)NH—, —C(O)O—, —O—, and —NH—.


SEQUENCE LISTING (22 sequences)

SEQ ID NO: 1 - protein, 26 aa, synthetic construct
EEEEEEEEEEEEEEEEGGGGGGRGDK

SEQ ID NO: 2 - protein, 26 aa, synthetic construct
RGDKGGGGGGEEEEEEEEEEEEEEEE

SEQ ID NO: 3 - protein, 22 aa, synthetic construct
GGGGGGEEEEEEEEEEEEEEEE

SEQ ID NO: 4 - protein, 33 aa, synthetic construct
GDPDLGDVDRGGGGGGGEEEEEEEEEEEEEEEE

SEQ ID NO: 5 - protein, 15 aa, synthetic construct
VTWFHAIHVSGTNGT

SEQ ID NO: 6 - protein, 8 aa, synthetic construct
VNFNFNGL

SEQ ID NO: 7 - skipped

SEQ ID NO: 8 - protein, 30 aa, synthetic construct
QDLFLPFFSNVTWFHAIHVSGTNGTKRFDN

SEQ ID NO: 9 - protein, 28 aa, synthetic construct
QDLFLPFFSNVTWFHAISGTNGTKRFDN

SEQ ID NO: 10 - protein, 30 aa, synthetic construct
QDLFLPFFSNVTWFHAIHVSGTNGTKRFAN

SEQ ID NO: 11 - protein, 30 aa, synthetic construct
QDLFLPFFSNVTWFHAIHVSGTNGTTRFDN

SEQ ID NO: 12 - protein, 28 aa, synthetic construct
QDLFLPFFSNVTWFHAISGTNGIKRFDN

SEQ ID NO: 13 - protein, 30 aa, synthetic construct
SGYFLPFQSNLTRYHLTLESITGRRIYFDN

SEQ ID NO: 14 - protein, 28 aa, synthetic construct
PGYFLPFGTNITRYQAQVINWTAIFFDN

SEQ ID NO: 15 - protein, 23 aa, synthetic construct
QDLFLPFYSNVTGFHTINHTFDN

SEQ ID NO: 16 - protein, 23 aa, synthetic construct
QDHFLPFDSNVTRFITFGLNFDN

SEQ ID NO: 17 - protein, 29 aa, synthetic construct
QGYFLPFYSNISWYYALTKTNGAEKRVDN

SEQ ID NO: 18 - protein, 33 aa, synthetic construct
KKSTNLVKNKCVNFNFNGLTGTGVLTESNKKFL

SEQ ID NO: 19 - protein, 33 aa, synthetic construct
KKSTNLVKNKCVNFNFNGLKGTGVLTESNKKFL

SEQ ID NO: 20 - protein, 34 aa, synthetic construct
KQSTELVKNKCVNFNFNGLTGTGVLTESTKRFLQ

SEQ ID NO: 21 - protein, 33 aa, synthetic construct
KQSTDLVKNKCVNFNFNGLTGTGVLTDSNKKFQ

SEQ ID NO: 22 - protein, 33 aa, synthetic construct
KLSTDLIKNQCVNFNFNGLTGTGVLTPSSKRFQ